(19) United States
(12) Patent Application Publication
(10) Pub. No.: US 2025/0257261 A1
(43) Pub. Date: Aug. 14, 2025

(54) PLURALITY OF LIGHT-EMITTING MATERIAL AND ORGANIC ELECTROLUMINESCENT DEVICE COMPRISING THE SAME

(71) Applicant: DuPont Specialty Materials Korea Ltd., Chungcheongnam-do (KR)

(72) Inventors: Chi-Sik KIM, Gyeonggi-do (KR); Dong-Hyung LEE, Gyeonggi-do (KR); Soo-Yong LEE, Gyeonggi-do (KR); Seung-Hoon YOO, Gyeonggi-do (KR)

(21) Appl. No.: 19/196,828

(22) Filed: May 2, 2025

Related U.S. Application Data
(63) Continuation of application No. 17/065,742, filed on Oct. 8, 2020, now abandoned.

(30) Foreign Application Priority Data
Oct. 18, 2019 (KR) 10-2019-0130130

(51) Int. Cl. C09K 11/06; C07C 15/28; C09K 11/02; H10K 50/11; H10K 85/60

(52) U.S. Cl. CPC C09K 11/06; C07C 15/28; C09K 11/02; H10K 85/626; H10K 85/658; C07C 2603/24; C09K 2211/1014; C09K 2211/1022; H10K 50/11

(57) ABSTRACT

The present disclosure relates to a plurality of light-emitting materials comprising at least one of first compounds and at least one of second compounds, wherein the first compound is represented by formula 1, and the second compound is represented by formula 2, and an organic electroluminescent device having high color purity and/or long lifespan properties can be provided by comprising the plurality of light-emitting materials.

### CLAIM OF BENEFIT OF PRIOR APPLICATION

This application is a continuation of U.S. patent application Ser. No. 17/065,742, filed Oct. 8, 2020, which is the Convention Filing of KR10-2019-0130130 filed Oct. 18, 2019, both of which are incorporated by reference herein in their entirety.

### TECHNICAL FIELD

The present disclosure relates to a plurality of light-emitting material and an organic electroluminescent device comprising the same.

### BACKGROUND ART

An electroluminescent device (EL device) is a self-light-emitting display device which has advantages in that it provides a wider viewing angle, a greater contrast ratio, and a faster response time. An organic EL device was first developed by Eastman Kodak in 1987, by using small aromatic diamine molecules and aluminum complexes as materials for forming a light-emitting layer [Appl. Phys. Lett. 51, 913, 1987].

An organic electroluminescent device (OLED) changes electric energy into light by applying electricity to an organic light-emitting material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer (containing host and dopant materials), an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material, an electron buffer material, a hole blocking material, an electron transport material, an electron injection material, etc., depending on its functions. In the OLED, due to an application of a voltage, holes are injected from the anode to the light-emitting layer, electrons are injected from the cathode to the light-emitting layer, and excitons of high energies are formed by a recombination of the holes and the electrons. From this energy, organic luminescent compounds reach an excited state, and light emission occurs as the excited energy state of the organic luminescent compounds returns to a ground state.

As a recently commercialized display element, in addition to OLEDs, there are light emitting diodes (LEDs), quantum dots (QDs), and the like, each of which has pros and cons. For example, QD has the advantage of obtaining high color purity by increasing the color reproduction rate, but has poor lifespan properties. In the case of OLEDs, lifespan properties are excellent, but the color purity is poor. There may be a way to change the dopant to improve color purity in OLEDs. For example, boron derivatives may be used as a dopant instead of pyrene derivatives in fluorescent blue light-emitting OLEDs. When a boron derivative is used, it is possible to increase color purity, which is considered to be due to a rigid structure. However, in the case of boron derivatives, there is a problem in that lifespan properties are generally lower than when pyrene derivatives are used. Accordingly, there is a technical need to improve lifespan properties when using a boron derivative as a dopant.

Meanwhile, Korean Patent Appl. Laid-Open No. 2017-0130434 discloses a combination comprising an anthracene derivative and a boron derivative. However, the development for improving performances of an OLED is still required. In addition, Korean Patent Appl. Laid-Open No. 2015-0010016 discloses anthracene derivative hosts, but fails to disclose specific examples of using the anthracene derivatives in combination with a boron derivative.

### DISCLOSURE OF INVENTION

### Technical Problem

The objective of the present disclosure is to provide an organic electroluminescent device having high color purity and/or long lifespan properties.

### Solution to Problem

The present inventors have tried to improve the performance of an organic electroluminescent device by combining a specific light-emitting material containing a boron derivative with a specific light-emitting material having long lifespan properties. As a result of intensive study, specifically, the present inventors have completed the present invention by finding that the above-described objective is achieved from a plurality of light-emitting materials comprising at least one of first compounds and at least one of second compounds, wherein the first compound is represented by the following formula 1, and the second compound is represented by the following formula 2:

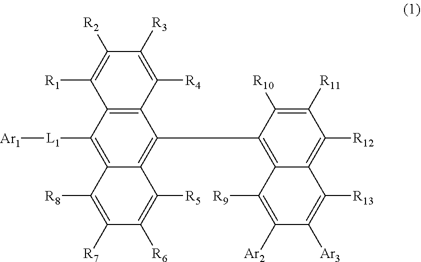

wherein,
L₁ represents a single bond, a substituted or unsubstituted (C6-C30)arylene, or a substituted or unsubstituted (5- to 30-membered)heteroarylene;
Ar₁ represents a substituted or unsubstituted (C6-C30)aryl, or a substituted or unsubstituted (5- to 30-membered)heteroaryl;
Ar₂ and Ar₃, each independently, represent hydrogen, deuterium, a substituted or unsubstituted (C6-C30)aryl, or a substituted or unsubstituted (5- to 30-membered)heteroaryl, with a proviso that Ar₂ and Ar₃ are not simultaneously hydrogen; and
R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;

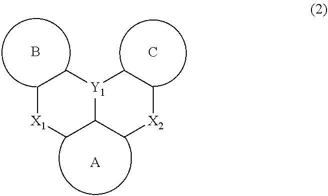

wherein,
ring A, ring B, and ring C, each independently, represent a substituted or unsubstituted (C6-C30)aryl, or a substituted or unsubstituted (3- to 50-membered)heteroaryl, wherein ring B and ring C may be linked to each other to form a ring;
Y₁ represents B;
X₁ and X₂, each independently, represent NR or O; and
R each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to at least one of ring A, ring B, and ring C to form a ring(s).

The compound represented by formula 1 contains a naphthalene substituted at 6- or 7-position, which is considered to reduce the interaction between hosts. It is not intended to be restricted by theory, however, as a result, it is believed that crystallinity decreases and amorphousness increases, which may have an effect of improving lifespan properties of the organic electroluminescent device.

### Advantageous Effects of Invention

By comprising the plurality of light-emitting materials according to the present disclosure, it is possible to provide an organic electroluminescent device having high color purity and/or long lifespan properties.

### MODE FOR THE INVENTION

Hereinafter, the present disclosure will be described in detail. However, the following description is intended to explain the disclosure, and is not meant in any way to restrict the scope of the disclosure.

The term “an organic electroluminescent material” in the present disclosure means a material that may be used in an organic electroluminescent device, and may comprise at least one compound. If necessary, the organic electroluminescent material may be comprised in any layers constituting an organic electroluminescent device. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of light-emitting materials” in the present disclosure means a host material(s) and/or a dopant material(s), comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of light-emitting materials of the present disclosure may be a combination of one or more host materials and one or more dopant materials, and may optionally further include a conventional material comprised in organic electroluminescent materials. The two or more compounds comprised in the plurality of light-emitting materials of the present disclosure may be included in one light-emitting layer or may be respectively included in different light-emitting layers by means of the methods used in the art. For example, the two or more compounds may be mixture-evaporated or co-evaporated, or individually deposited.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7, preferably 5 to 7 ring backbone atoms, and including at least one heteroatom selected from the group consisting of B, N, O, S, Si, and P, preferably at least one heteroatom selected from the group consisting of O, S, and N. The above heterocycloalkyl may include tetrahydrofuran, pyrrolidine, thiolane, tetrahydropyran, etc. The term “(C6-C30)aryl(ene)” is meant to be a monocyclic or fused ring radical derived from an aromatic hydrocarbon having 6 to 30 ring backbone carbon atoms, which may be partially saturated. The number of ring backbone carbon atoms is preferably 6 to 25, and more preferably 6 to 18. The above aryl comprises those having a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, etc.

The term “(3- to 50-membered)heteroaryl(ene)” is an aryl(ene) having 3 to 50 ring backbone atoms, in which the number of ring backbone atoms is preferably 5 to 25, and including at least one, preferably 1 to 4 heteroatom(s) selected from the group consisting of B, N, O, S, Si, and P. The above heteroaryl(ene) may be a monocyclic ring or a fused ring condensed with at least one benzene ring, and may be partially saturated. In addition, the above heteroayl(ene) comprises the form in which at least one heteroaryl or aryl group is linked to a heteroaryl group via a single bond(s), and also comprises those having a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, naphthobenzofuranyl, naphthobenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” means that a hydrogen atom in a certain functional group is replaced with another atom or another functional group, i.e., a substituent. In the formulas of the present disclosure, the substituents of the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, or the substituted alkylarylamino, each independently, may be at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with at least one of a (C1-C30)alkyl(s), a (C6-C30)aryl(s) and a di(C6-C30)arylamino(s); a (C6-C30)aryl unsubstituted or substituted with at least one of a (C1-C30)alkyl(s), a (5- to 30-membered)heteroaryl(s) and a di(C6-C30)arylamino(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with at least one of a (C1-C30)alkyl(s), a (5- to 30-membered)heteroaryl(s) and a di(C6-C30)arylamino(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. Preferably, the substituents, each independently, are at least one selected from the group consisting of deuterium; a (C1-C20)alkyl; a (5- to 20-membered)heteroaryl unsubstituted or substituted with a (C1-C20)alkyl(s); a (C6-C25)aryl unsubstituted or substituted with at least one of a (C1-C20)alkyl(s), a (5- to 20-membered)heteroaryl(s) and a di(C6-C25)arylamino(s); and a mono- or di-(C6-C25)arylamino unsubstituted or substituted with at least one of a (C1-C20)alkyl(s), a (5- to 25-membered)heteroaryl(s) and a di(C6-C25)arylamino(s). More preferably, the substituents, each independently, are at least one selected from the group consisting of deuterium; a (C1-C10)alkyl; a (5- to 20-membered)heteroaryl unsubstituted or substituted with a (C1-C10)alkyl(s); a (C6-C18)aryl unsubstituted or substituted with at least one of a (C1-C10)alkyl(s), a (5- to 20-membered)heteroaryl(s) and a di(C6-C18)arylamino(s); and a mono- or di-(C6-C18)arylamino unsubstituted or substituted with at least one of a (C1-C10)alkyl(s), a (5- to 20-membered)heteroaryl(s) and a di(C6-C18)arylamino(s). For example, the substituents, each independently, may be at least one selected from the group consisting of a methyl; tert-butyl; a phenyl unsubstituted or substituted with at least one of a carbazolyl(s), a dibenzofuranyl(s), a methyl(s), a diphenylamino(s), a phenoxazinyl(s), a phenothiazinyl(s), and an acridinyl(s) substituted with a methyl(s); a biphenyl; a terphenyl; a triphenylenyl; a carbazolyl; a phenoxazinyl; a phenothiazinyl; an acridinyl substituted with a methyl(s); a xanthenyl substituted with a methyl(s); a diphenylamino unsubstituted or substituted with a methyl(s) and/or a diphenylamino(s); a phenylnaphthylamino; and a phenylamino substituted with a phenylcarbazolyl(s) and/or a dibenzofuranyl(s).

In the formulas of the present disclosure, in case a ring is formed from a linkage with an adjacent substituent, the ring may be a substituted or unsubstituted mono- or polycyclic (3- to 30-membered) alicyclic or aromatic ring, or the combination thereof. In addition, the formed ring may contain at least one heteroatom selected from B, N, O, S, Si, and P, preferably at least one heteroatom selected from N, O, and S. According to one embodiment of the present disclosure, the number of ring backbone atoms is (5- to 20-membered), and according to another embodiment of the present disclosure, the number of ring backbone atoms is (5- to 15-membered). The above fused ring may include the form of a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring.

In the formulas of the present disclosure, the heteroaryl, the heteroarylene, and the heterocycloalkyl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, the plurality of light-emitting materials according to one embodiment will be explained.

The plurality of light-emitting materials according to one embodiment comprises at least one first compound represented by formula 1 and at least one second compound represented by formula 2. Specifically, the present disclosure provides an organic electroluminescent device exhibiting high color purity and/or long lifespan properties by comprising the plurality of light-emitting materials in at least one organic layer, for example, at least one light-emitting layer of the organic electroluminescent device.

According to one embodiment, the present disclosure provides a host/dopant combination, i.e., a combination of the host compound represented by formula 1 and the dopant compound represented by formula 2. Also, the present disclosure provides an organic electroluminescent device comprising the host/dopant combination.

The light-emitting material according to one embodiment includes at least one anthracene derivative represented by formula 1. According to one embodiment, the compound represented by formula 1 may be a fluorescent host, for example, a blue light-emitting fluorescent host.

In formula 1, L₁ represents a single bond, a substituted or unsubstituted (C6-C30)arylene, or a substituted or unsubstituted (5- to 30-membered)heteroarylene. Preferably, L₁ may represent a single bond, a substituted or unsubstituted (C6-C18)arylene, or a substituted or unsubstituted (5- to 18-membered)heteroarylene, more preferably, a single bond, an unsubstituted (C6-C18)arylene, or an unsubstituted (5- to 18-membered)heteroarylene.

According to one embodiment of the present disclosure, L₁ may represent a single bond, or any one selected from the following Group 1.

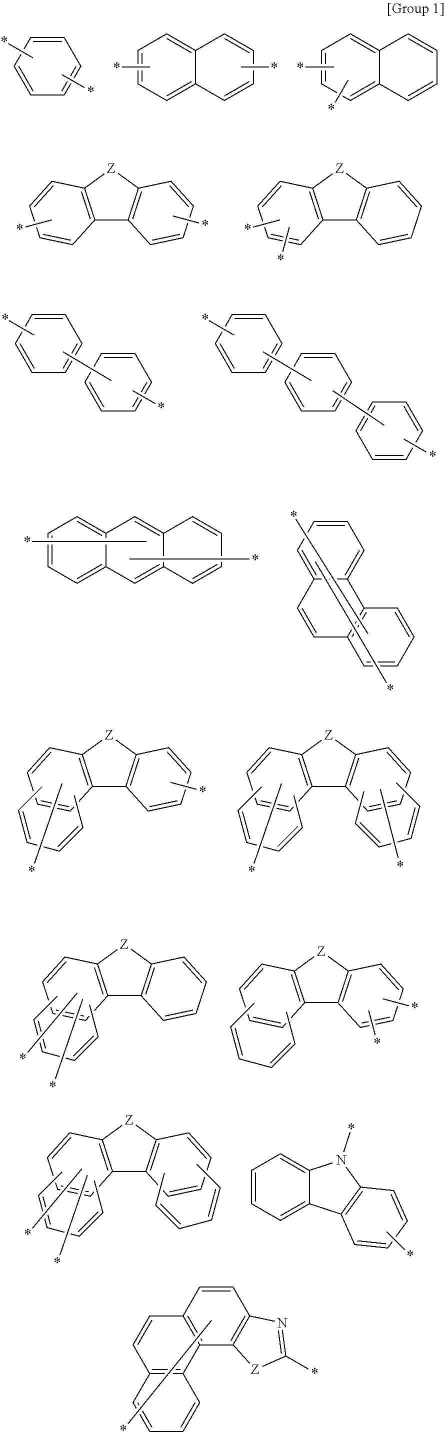

In Group 1, Z represents O, S, NR₁₀₁, CR₁₀₂R₁₀₃, or SiR₁₀₄R₁₀₅. Preferably, Z may represent NR₁₀₁ or O.

In Group 1, R₁₀₁ to R₁₀₅, each independently, represent a substituted or unsubstituted (C1-C30)alkyl, a substituted or unsubstituted (C6-C30)aryl, or a substituted or unsubstituted (3- to 30-membered)heteroaryl, or may be linked to an adjacent substituent to form a ring(s).

In Group 1, * represents a bonding site with the anthracene backbone and Ar₁.

According to one embodiment of the present disclosure, L₁ may represent a single bond, a phenylene, a naphthylene, a biphenylene, a carbazolylene, a phenanthrooxazolylene, etc.

In formula 1, Ar₁ represents a substituted or unsubstituted (C6-C30)aryl or a substituted or unsubstituted (5- to 30-membered)heteroaryl. Preferably, Ar₁ may represent a substituted or unsubstituted (C6-C30)aryl or a substituted or unsubstituted (5- to 18-membered)heteroaryl, more preferably, a (C6-C25)aryl unsubstituted or substituted with a (C1-C6) alkyl(s) and/or a (C6-C12)aryl(s), or a (5- to 18-membered)heteroaryl unsubstituted or substituted with a (C6-C12)aryl(s).

In formula 1, Ar₂ and Ar₃, each independently, represent hydrogen, deuterium, a substituted or unsubstituted (C6-C30)aryl, or a substituted or unsubstituted (5- to 30-membered)heteroaryl, with a proviso that Ar₂ and Ar₃ are not simultaneously hydrogen. Preferably, Ar₂ and Ar₃, each independently, may represent hydrogen, a substituted or unsubstituted (C6-C18)aryl, or a substituted or unsubstituted (5- to 18-membered)heteroaryl, more preferably, each independently, hydrogen, a (C6-C18)aryl unsubstituted or substituted with a (C6-C12) aryl(s), or an unsubstituted (5- to 18-membered)heteroaryl.

According to one embodiment of the present disclosure, Ar₁ is selected from the substituents listed in the following Group 2, and Ar₂ and Ar₃, each independently, represent hydrogen or deuterium, or selected from the substituents listed in the following Group 2, with a proviso that Ar₂ and Ar₃ are not simultaneously hydrogen.

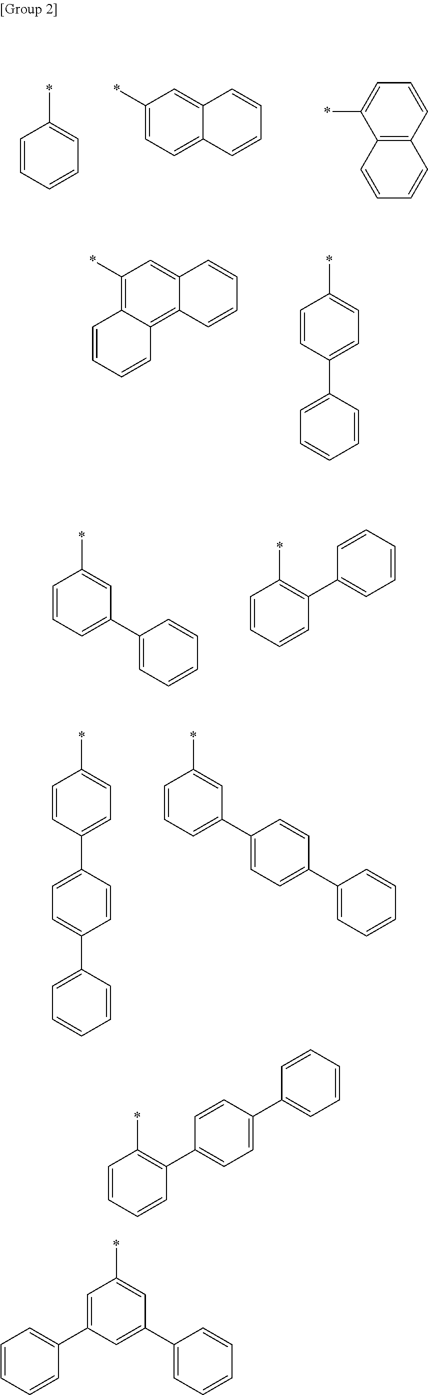

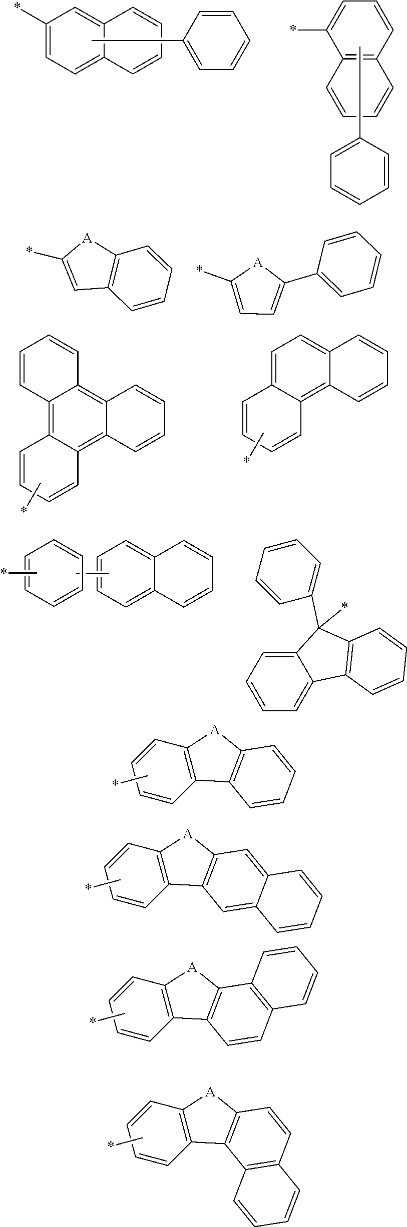

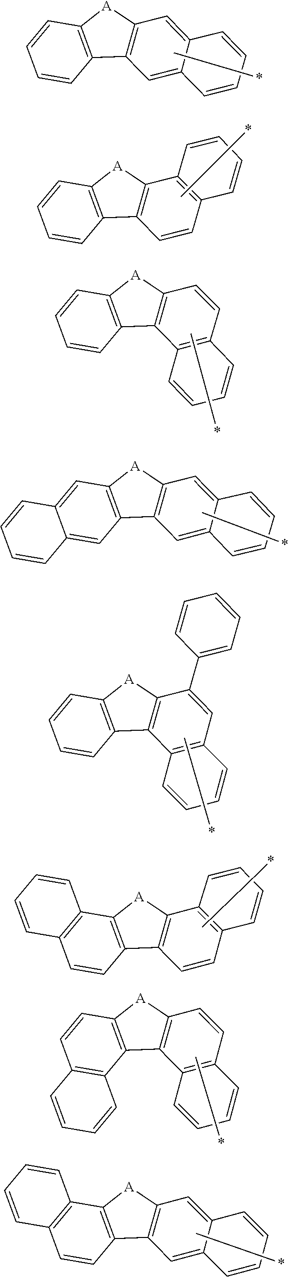

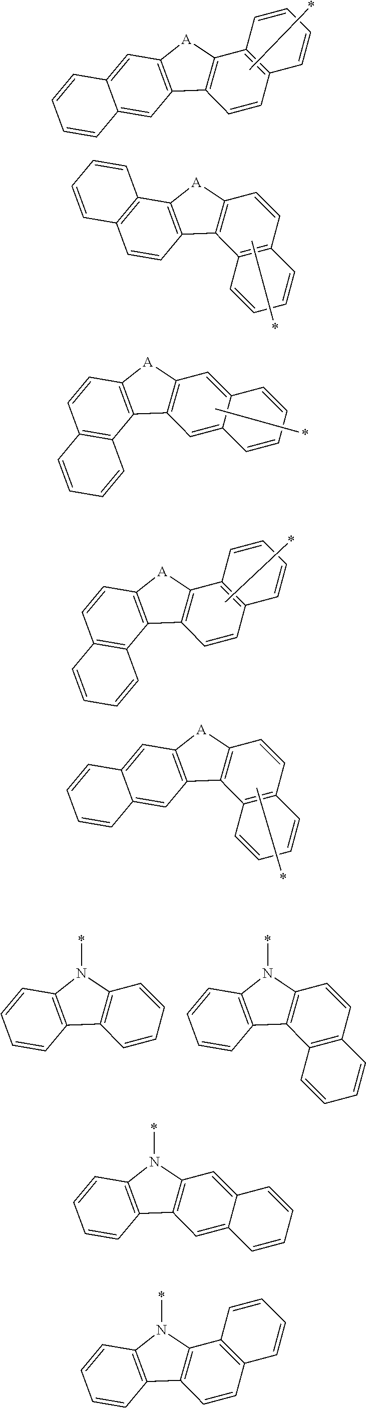

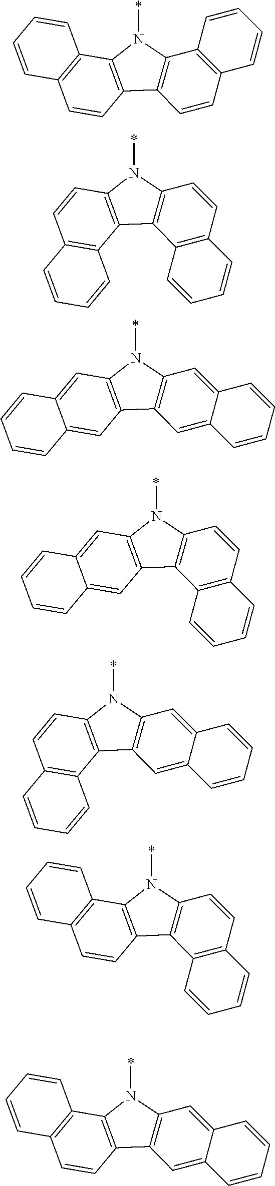

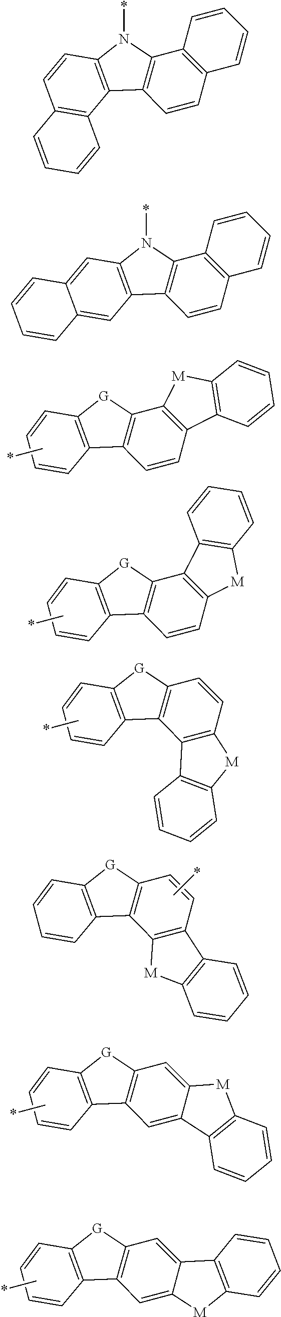

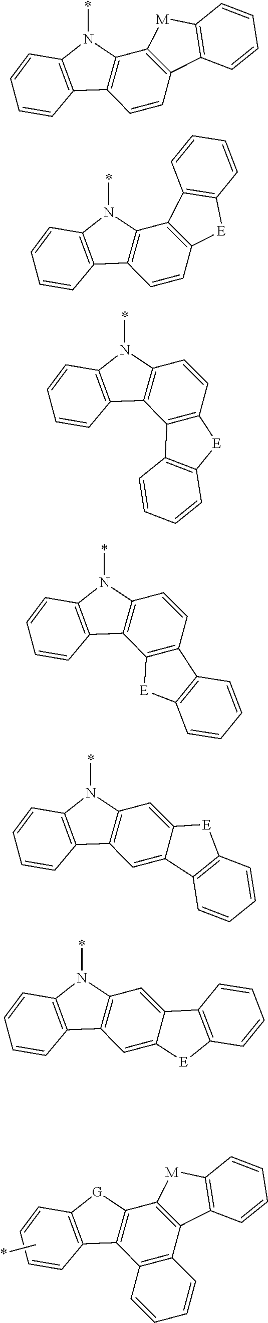

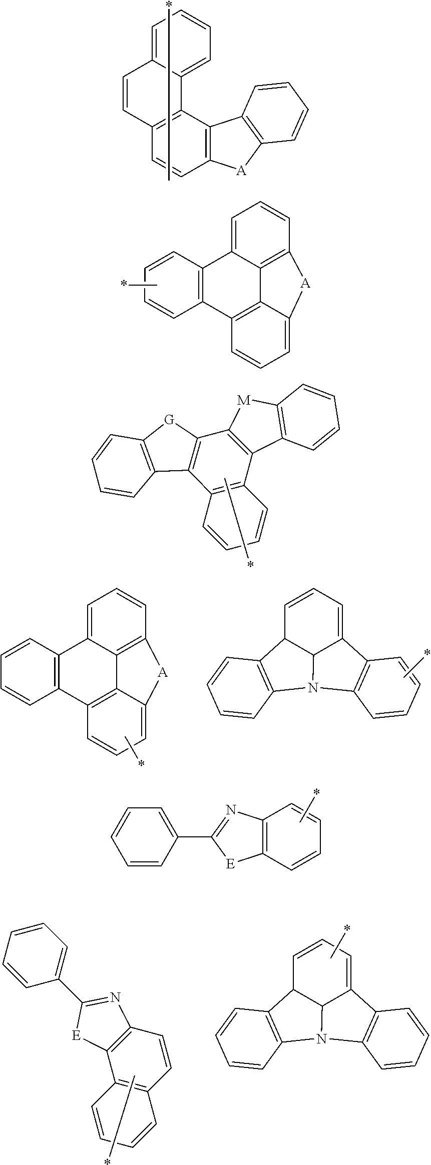

In Group 2, A, G, E, and M, each independently, represent O, S, NR₁₀₆, CR₁₀₇R₁₀₈, or SiR₁₀₉R₁₁₀.

R₁₀₆ to R₁₁₀, each independently, represent a substituted or unsubstituted (C1-C30)alkyl, a substituted or unsubstituted (C6-C30)aryl, or a substituted or unsubstituted (3- to 30-membered)heteroaryl; or may be linked to an adjacent substituent to form a ring(s). Preferably, R₁₀₆ to R₁₁₀, each independently, represent a substituted or unsubstituted (C1-C6)alkyl, a substituted or unsubstituted (C6-C12)aryl, or a substituted or unsubstituted (5- to 18-membered)heteroaryl, or may be linked to an adjacent substituent to form a ring(s). More preferably, R₁₀₆ to R₁₁₀, each independently, represent an unsubstituted (C1-C6)alkyl or an unsubstituted (C6-C12)aryl, or may be linked to an adjacent substituent to form a ring(s). For example, R₁₀₆ to R₁₁₀, each independently, may represent an unsubstituted methyl, an unsubstituted phenyl, etc., or R₁₀₇ and R₁₀₈ are linked to each other to form a ring such as fluorene.

In Group 2, * represents a bonding site with the anthracene backbone or L₁.

For example, Ar₁ may represent a phenyl, a naphthyl, a biphenyl, a phenanthrenyl, a terphenyl, a triphenylenyl, a spirobifluorenyl, a phenylfluorenyl, a diphenylfluorenyl, a diphenylbenzofluorenyl, a dimethylfluorenyl, a dimethylbenzofluorenyl, a dibenzofuranyl, a dibenzothiophenyl, a carbazolyl, a benzonaphthofuranyl, a benzonaphthothiophenyl, etc.

For example, Ar₂ and Ar₃, each independently, may represent hydrogen, deuterium, a phenyl, a naphthyl, a biphenyl, a phenanthrenyl, a naphthylphenyl, a dibenzofuranyl, a dibenzothiophenyl, a phenylcarbazolyl, a phenylphenanthrooxazolyl, etc.

In formula 1, R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino. Preferably, R₁ to R₁₃, each independently, represent hydrogen or deuterium, more preferably, may represent hydrogen.

The compound represented by formula 1 may be at least one selected from the following compounds, but is not limited thereto.

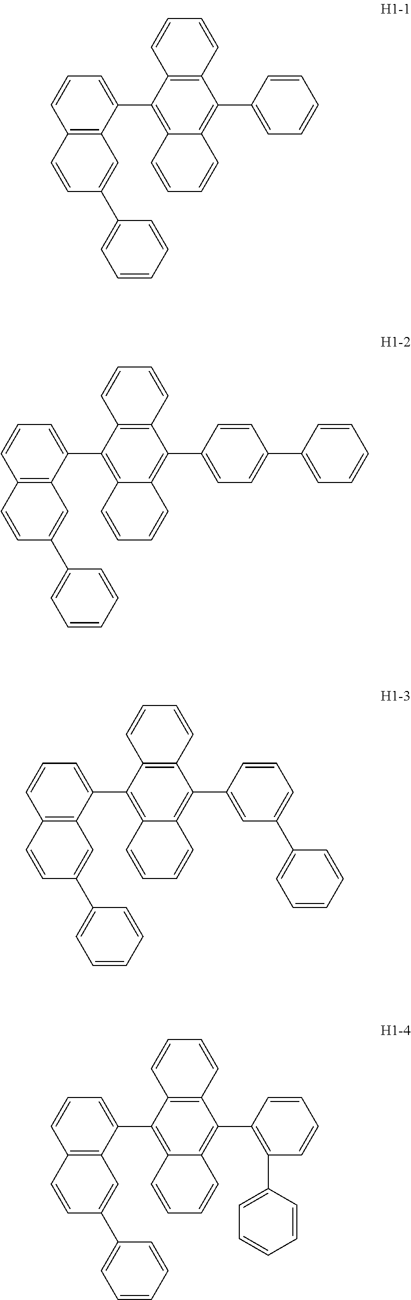

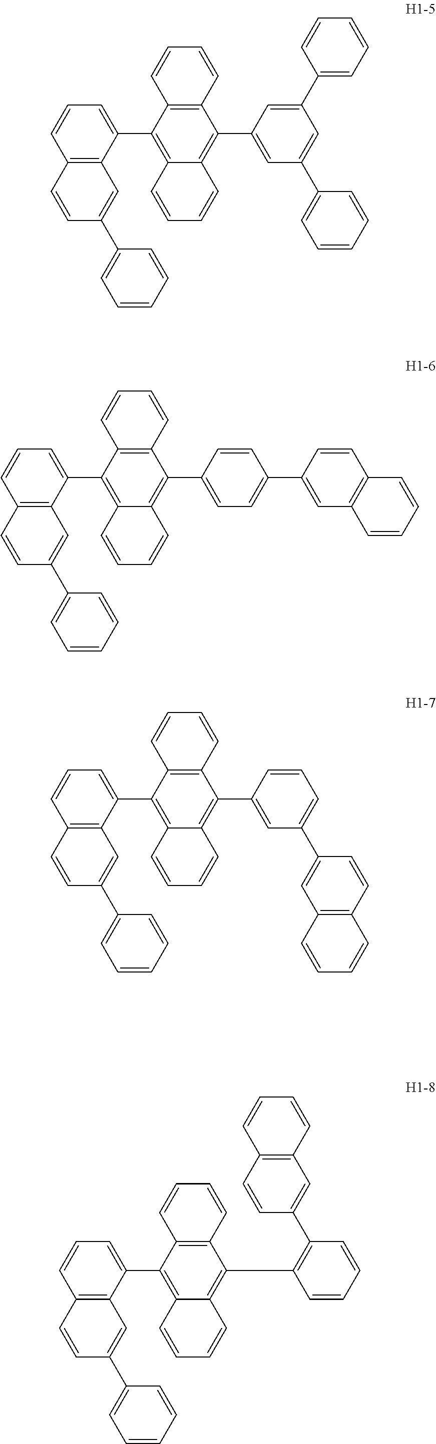

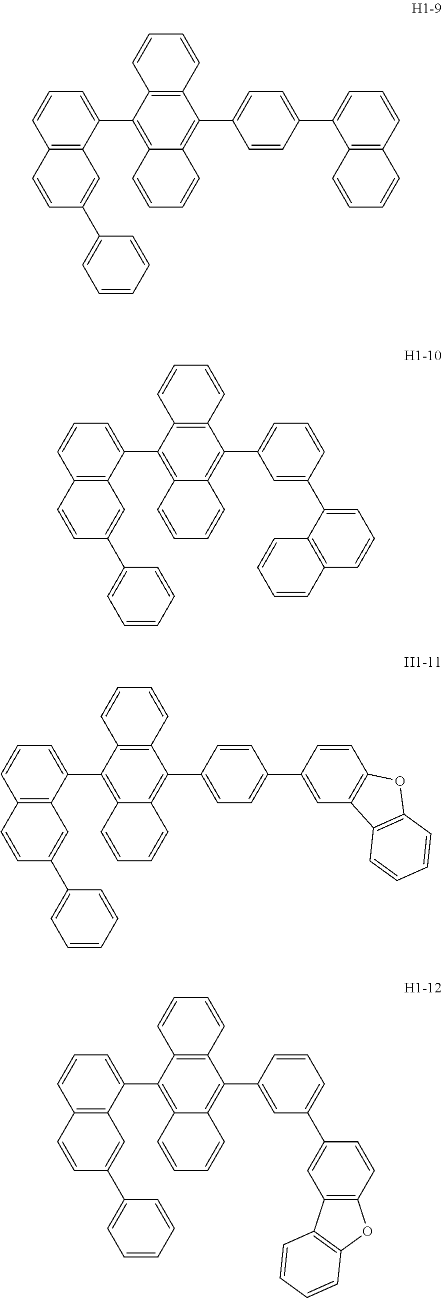

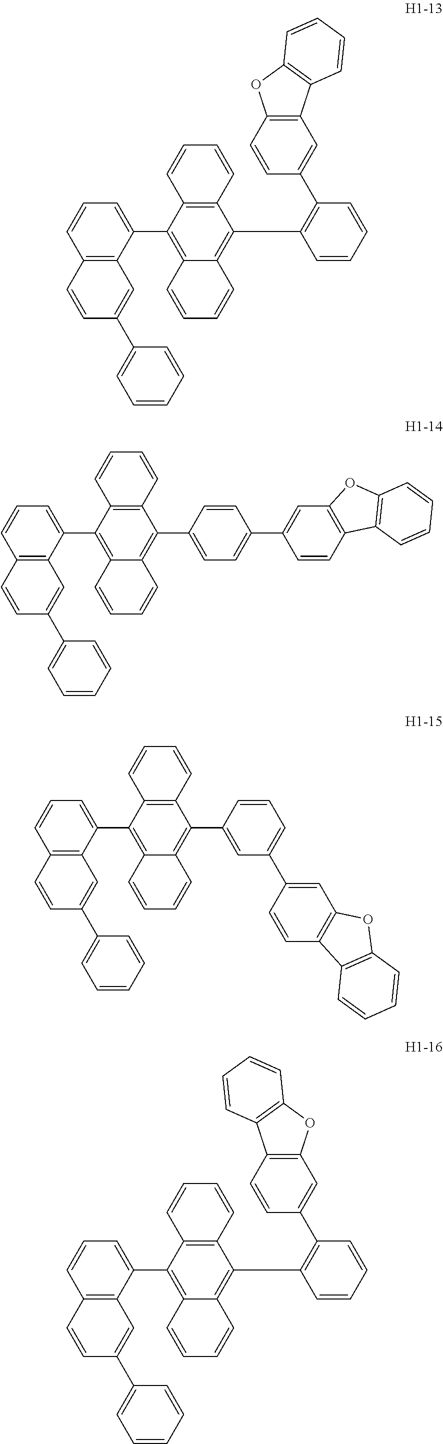 [SMILES: c1ccc(-c2ccc3cccc(-c4c5ccccc5c(Cc5ccccc5-c5ccc6oc7ccccc7c6c5)c5ccccc45)c3c2)cc1 ; c1ccc(-c2ccc3cccc(-c4c5ccccc5c(-c5ccc(-c6ccc7c(c6)oc6ccccc67)cc5)c5ccccc45)c3c2)cc1 ; c1ccc(-c2ccc3cccc(-c4c5ccccc5c(-c5cccc(-c6ccc7c(c6)oc6ccccc67)c5)c5ccccc45)c3c2)cc1 ; c1ccc(-c2ccc3cccc(-c4c5ccccc5c(Cc5ccccc5-c5ccc6c(c5)oc5ccccc56)c5ccccc45)c3c2)cc1]

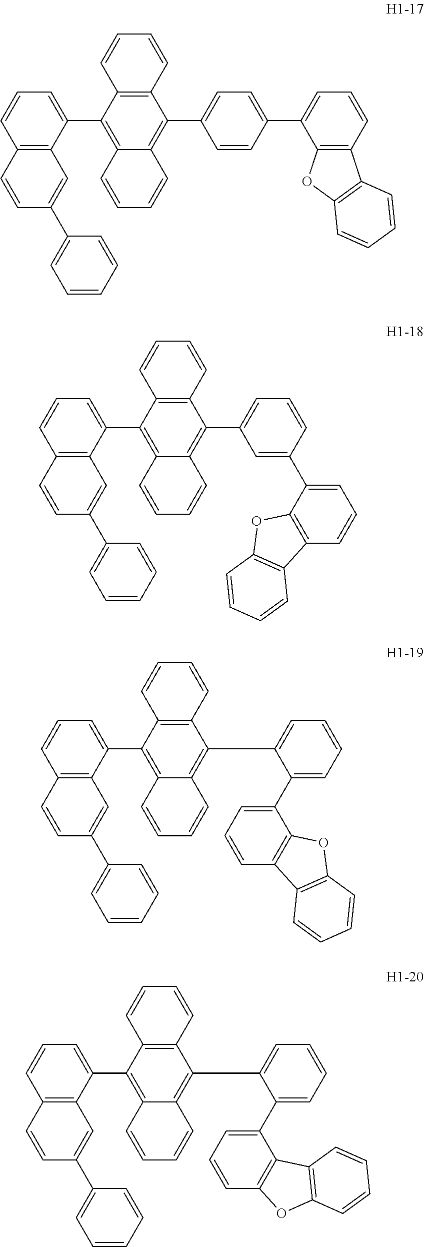 [SMILES: c1ccc(-c2ccc3cccc(-c4c5ccccc5c(-c5ccc(-c6cccc7c6oc6ccccc67)cc5)c5ccccc45)c3c2)cc1 ; c1ccc(-c2ccc3cccc(-c4c5ccccc5c(-c5cccc(-c6cccc7c6oc6ccccc67)c5)c5ccccc45)c3c2)cc1 ; c1ccc(-c2ccc3cccc(-c4c5ccccc5c(Cc5ccccc5-c5cccc6c5oc5ccccc56)c5ccccc45)c3c2)cc1 ; c1ccc(-c2ccc3cccc(-c4c5ccccc5c(Cc5ccccc5-c5cccc6oc7ccccc7c56)c5ccccc45)c3c2)cc1]

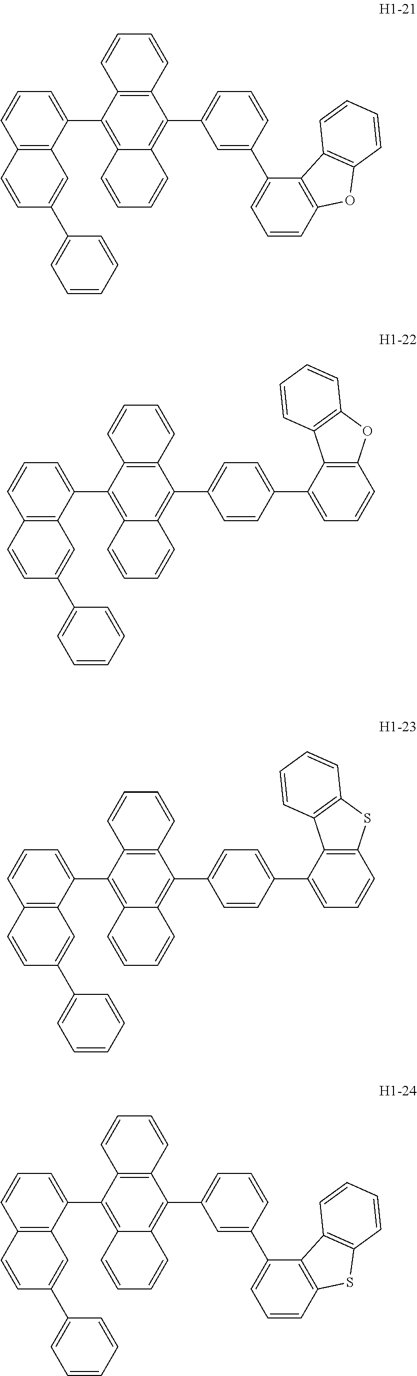 [SMILES: c1ccc(-c2ccc3cccc(-c4c5ccccc5c(-c5cccc(-c6cccc7oc8ccccc8c67)c5)c5ccccc45)c3c2)cc1 ; c1ccc(-c2ccc3cccc(-c4c5ccccc5c(-c5ccc(-c6cccc7oc8ccccc8c67)cc5)c5ccccc45)c3c2)cc1 ; c1ccc(-c2ccc3cccc(-c4c5ccccc5c(-c5ccc(-c6cccc7sc8ccccc8c67)cc5)c5ccccc45)c3c2)cc1 ; c1ccc(-c2ccc3cccc(-c4c5ccccc5c(-c5cccc(-c6cccc7sc8ccccc8c67)c5)c5ccccc45)c3c2)cc1]

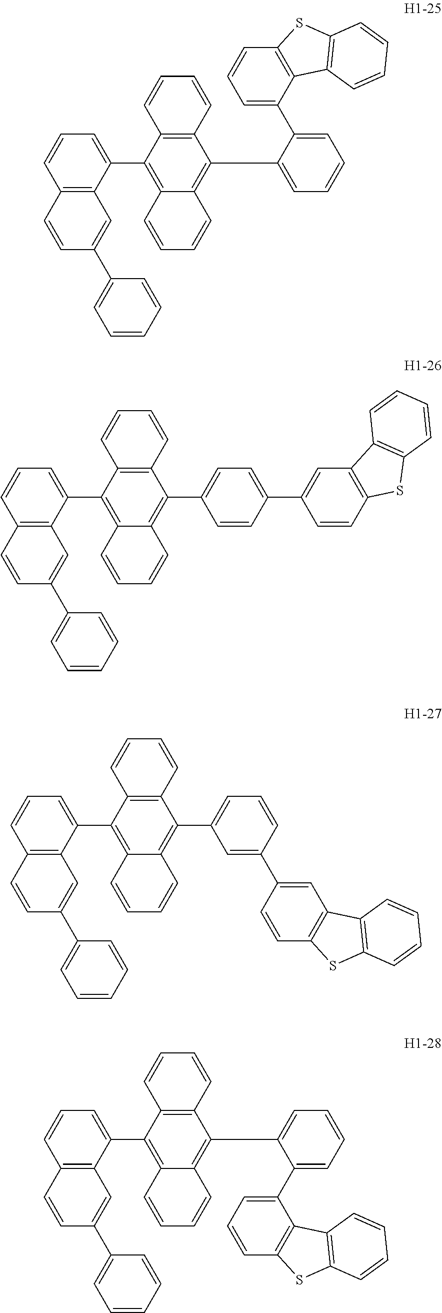 [SMILES: c1ccc(-c2ccc3cccc(-c4c5ccccc5c(Cc5ccccc5-c5cccc6sc7ccccc7c56)c5ccccc45)c3c2)cc1 ; c1ccc(-c2ccc3cccc(-c4c5ccccc5c(-c5ccc(-c6ccc7sc8ccccc8c7c6)cc5)c5ccccc45)c3c2)cc1 ; c1ccc(-c2ccc3cccc(-c4c5ccccc5c(-c5cccc(-c6ccc7sc8ccccc8c7c6)c5)c5ccccc45)c3c2)cc1 ; c1ccc(-c2ccc3cccc(-c4c5ccccc5c(Cc5ccccc5-c5cccc6sc7ccccc7c56)c5ccccc45)c3c2)cc1]

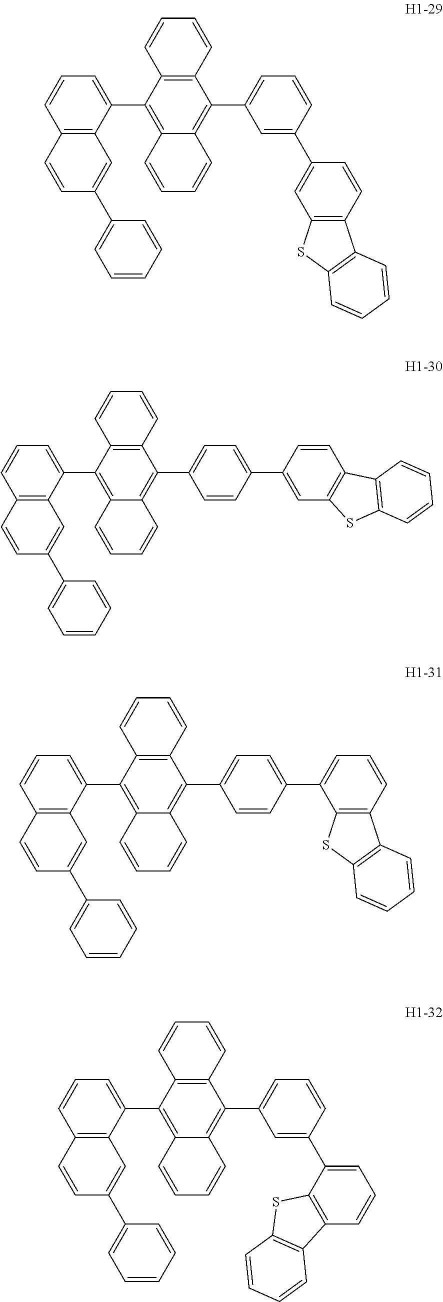 [SMILES: c1ccc(-c2ccc3cccc(-c4c5ccccc5c(-c5cccc(-c6ccc7c(c6)sc6ccccc67)c5)c5ccccc45)c3c2)cc1 ; c1ccc(-c2ccc3cccc(-c4c5ccccc5c(-c5ccc(-c6ccc7c(c6)sc6ccccc67)cc5)c5ccccc45)c3c2)cc1 ; c1ccc(-c2ccc3cccc(-c4c5ccccc5c(-c5ccc(-c6cccc7c6sc6ccccc67)cc5)c5ccccc45)c3c2)cc1 ; c1ccc(-c2ccc3cccc(-c4c5ccccc5c(-c5cccc(-c6cccc7c6sc6ccccc67)c5)c5ccccc45)c3c2)cc1]

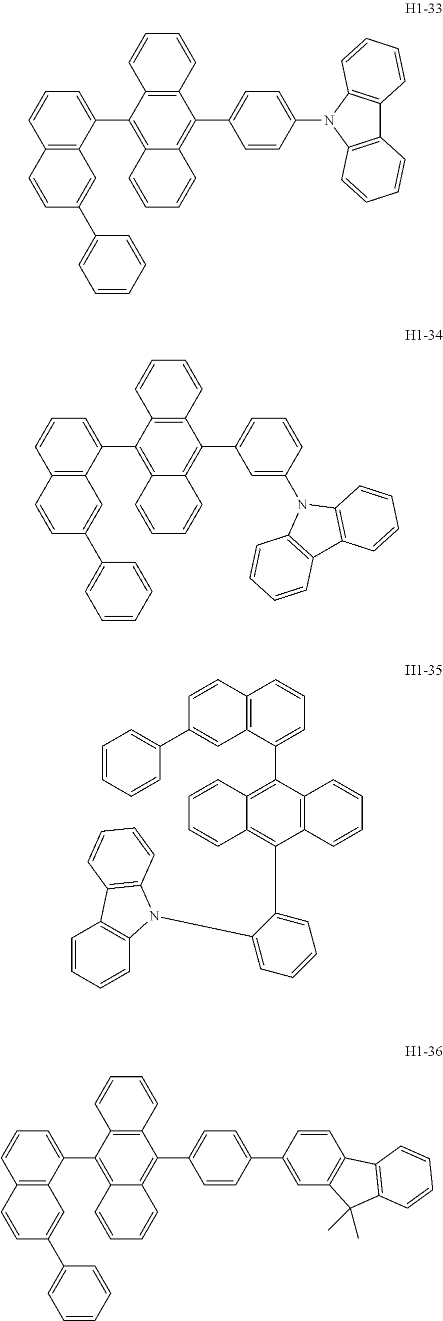

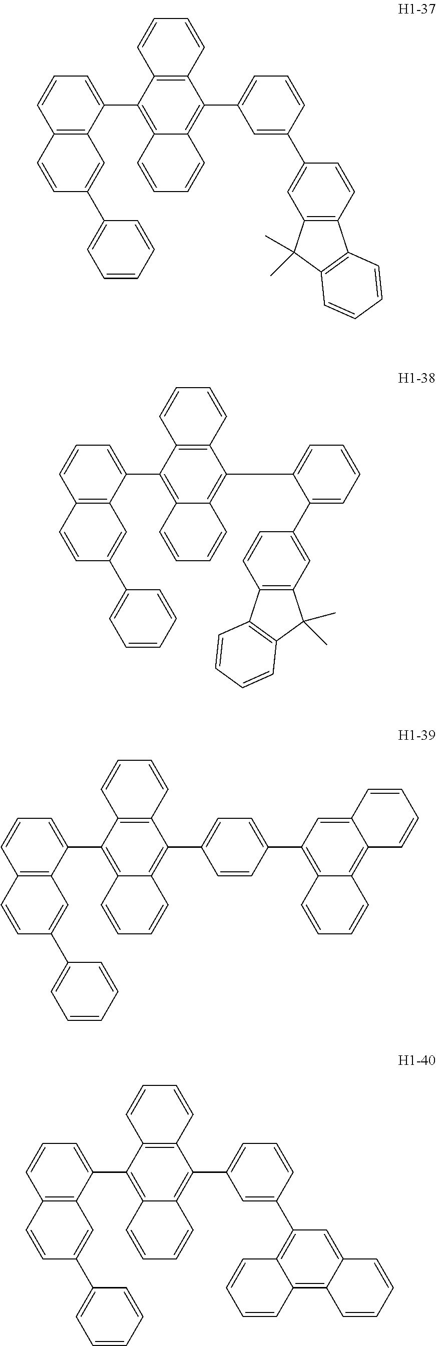

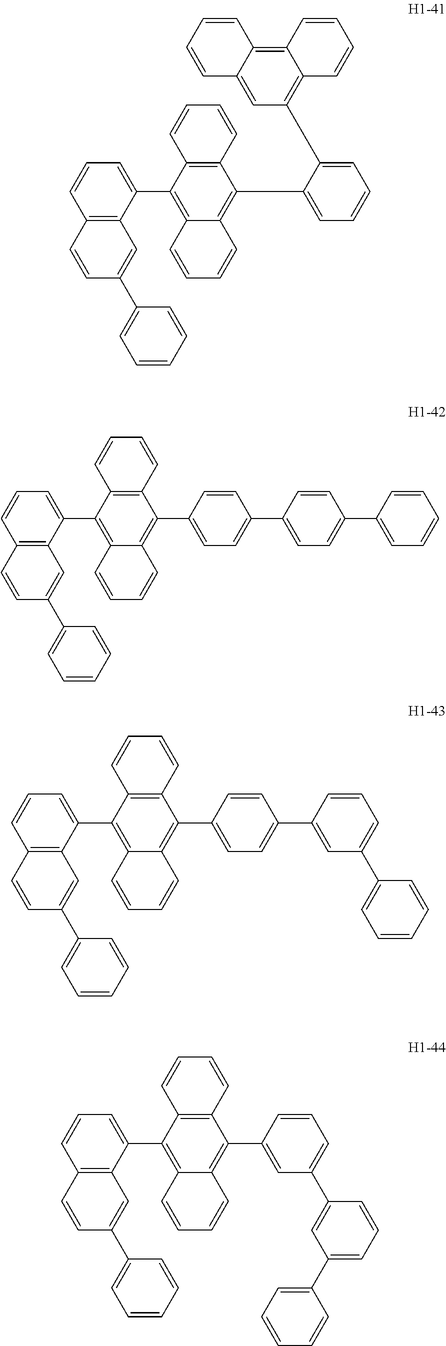

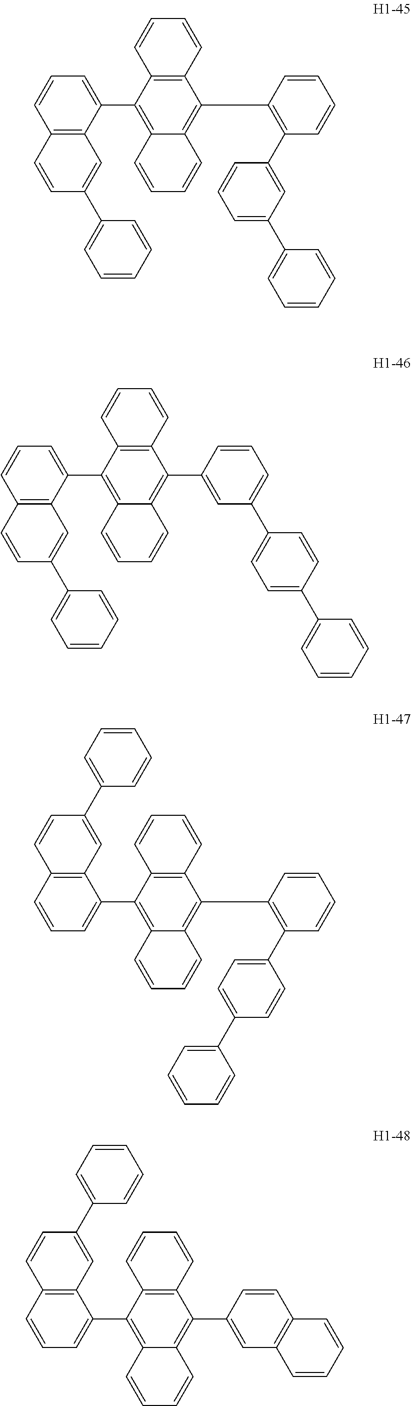

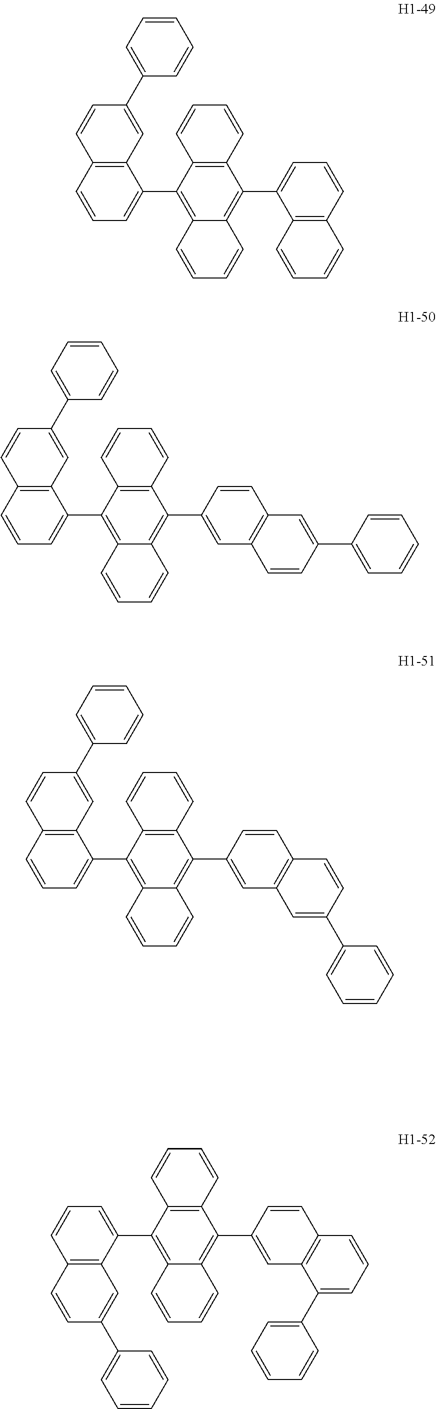

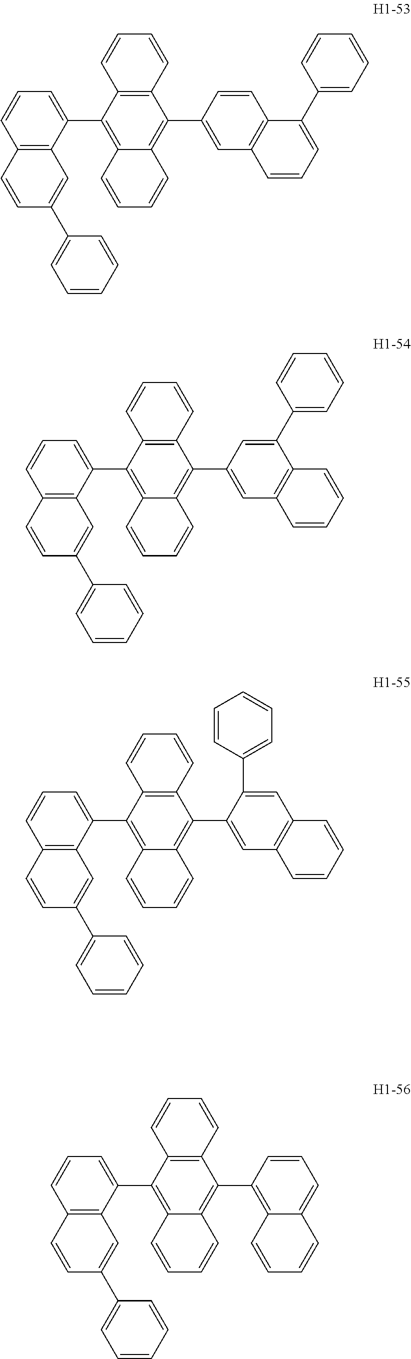

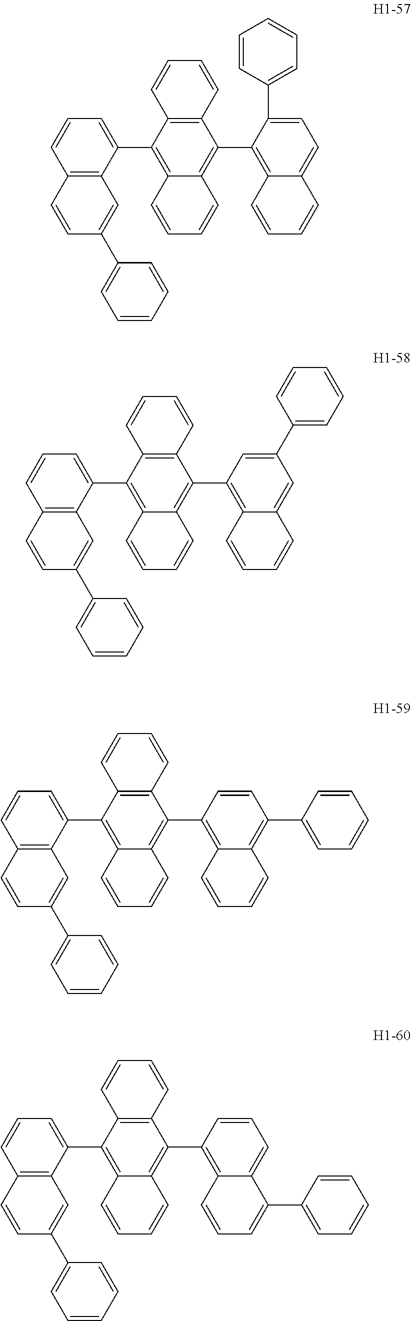

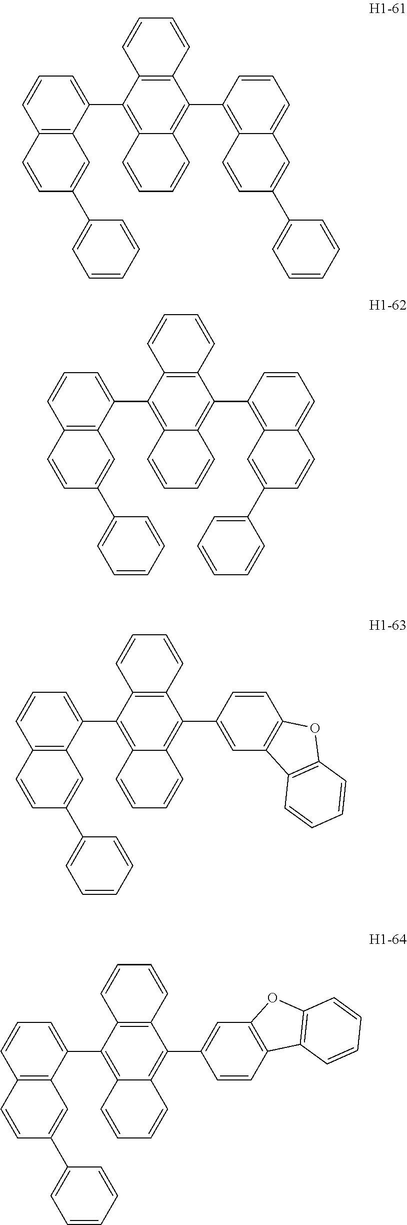

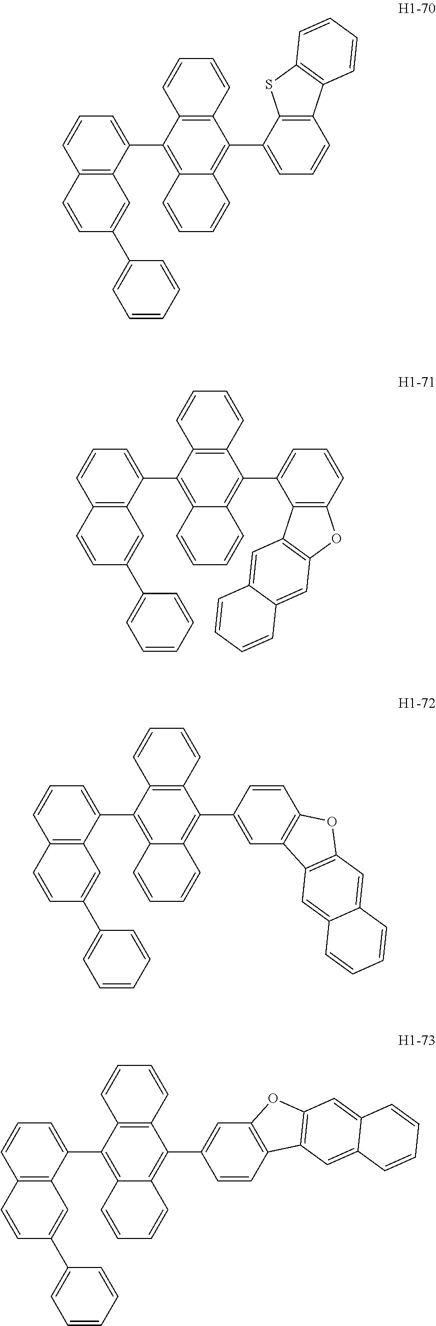 [SMILES: c1ccc(-c2ccc3cccc(-c4c5ccccc5c(-c5cccc6c5sc5ccccc56)c5ccccc45)c3c2)cc1 ; c1ccc(-c2ccc3cccc(-c4c5ccccc5c(-c5cccc6oc7cc8ccccc8cc7c56)c5ccccc45)c3c2)cc1 ; c1ccc(-c2ccc3cccc(-c4c5ccccc5c(-c5ccc6oc7cc8ccccc8cc7c6c5)c5ccccc45)c3c2)cc1 ; c1ccc(-c2ccc3cccc(-c4c5ccccc5c(-c5ccc6c(c5)oc5cc7ccccc7cc56)c5ccccc45)c3c2)cc1]

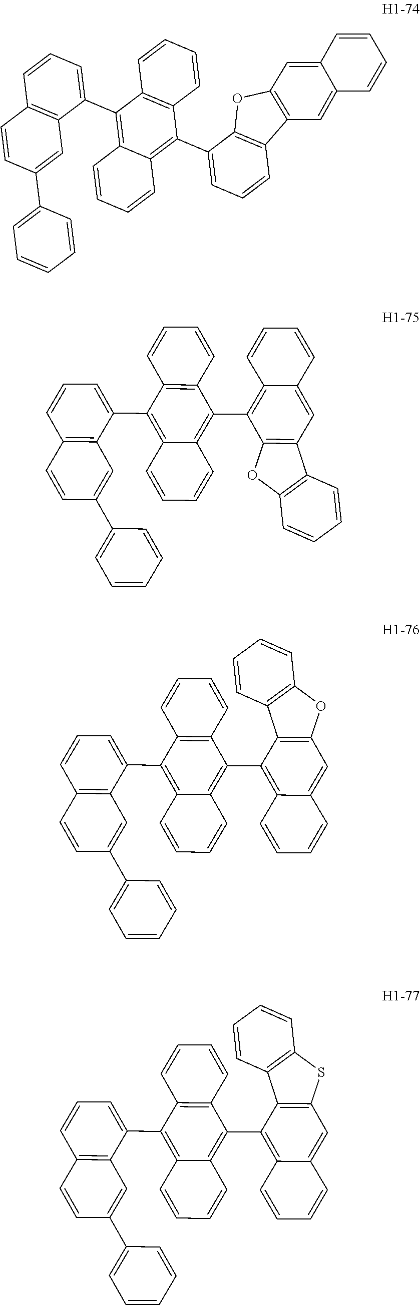 [SMILES: c1ccc(-c2ccc3cccc(-c4c5ccccc5c(-c5cccc6c5oc5cc7ccccc7cc56)c5ccccc45)c3c2)cc1 ; c1ccc(-c2ccc3cccc(-c4c5ccccc5c(-c5c6ccccc6cc6c5oc5ccccc56)c5ccccc45)c3c2)cc1 ; c1ccc(-c2ccc3cccc(-c4c5ccccc5c(-c5c6ccccc6cc6oc7ccccc7c56)c5ccccc45)c3c2)cc1 ; c1ccc(-c2ccc3cccc(-c4c5ccccc5c(-c5c6ccccc6cc6sc7ccccc7c56)c5ccccc45)c3c2)cc1]

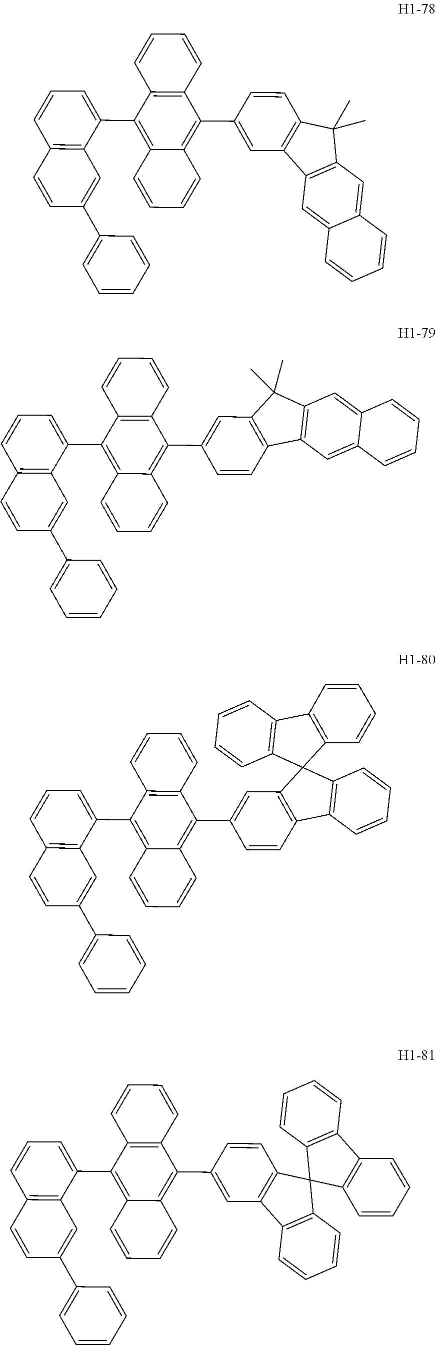

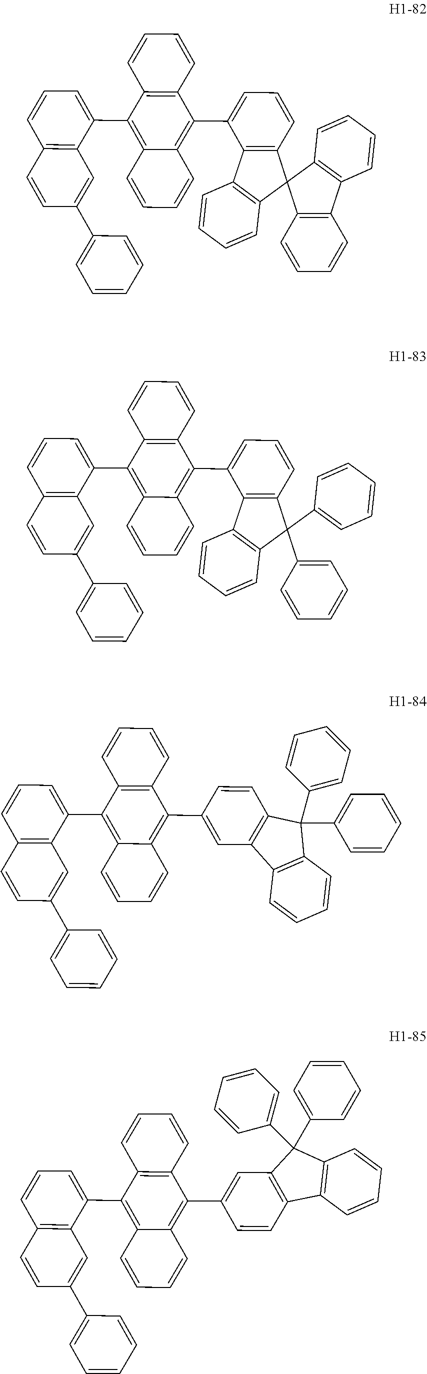

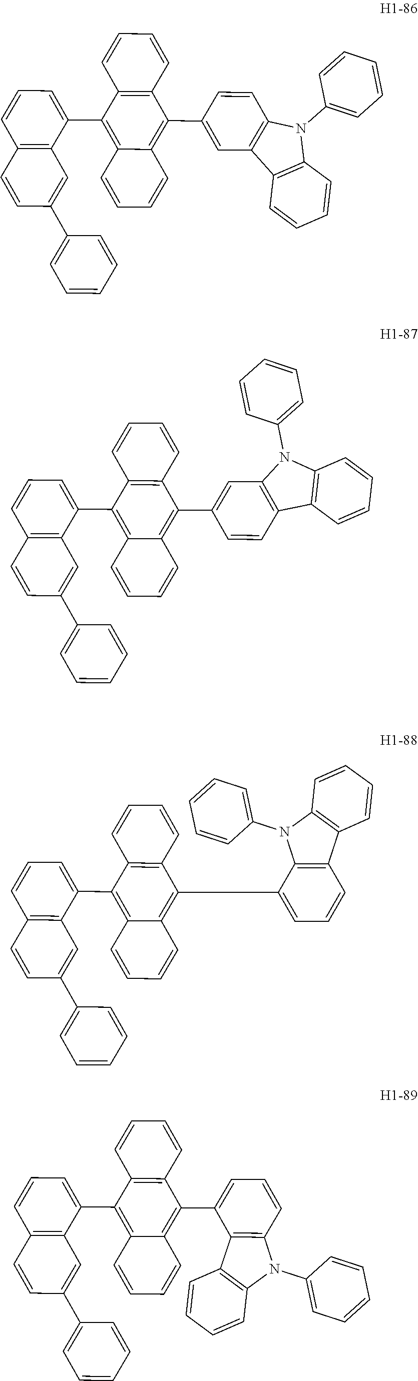 [SMILES: c1ccc(-c2ccc3cccc(-c4c5ccccc5c(-c5ccc6c(c5)c5ccccc5n6-c5ccccc5)c5ccccc45)c3c2)cc1 ; c1ccc(-c2ccc3cccc(-c4c5ccccc5c(-c5ccc6c7ccccc7n(-c7ccccc7)c6c5)c5ccccc45)c3c2)cc1 ; c1ccc(-c2ccc3cccc(-c4c5ccccc5c(CCc5cccc6c7ccccc7n(-c7ccccc7)c56)c5ccccc45)c3c2)cc1 ; c1ccc(-c2ccc3cccc(-c4c5ccccc5c(-c5cccc6c5c5ccccc5n6-c5ccccc5)c5ccccc45)c3c2)cc1]

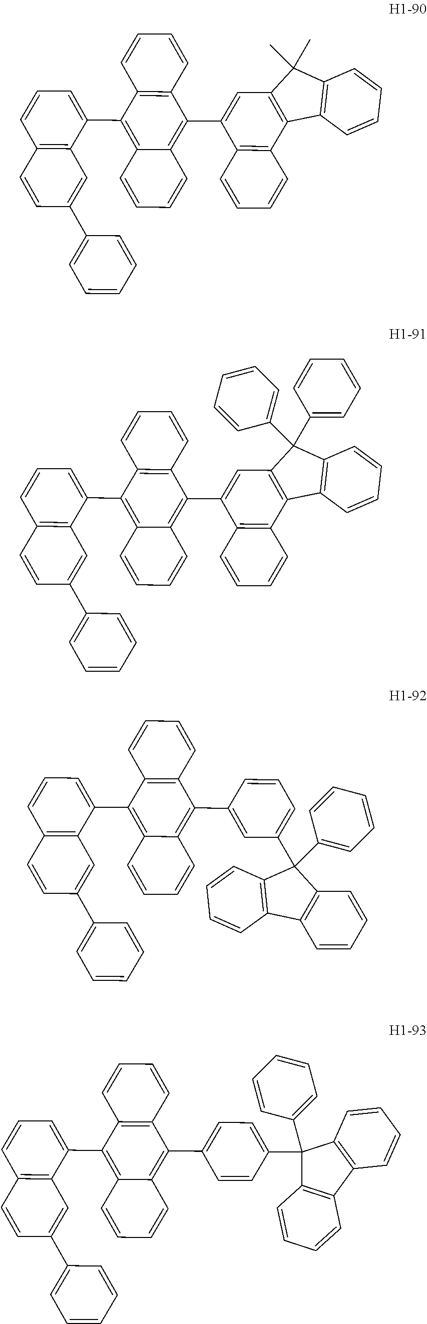

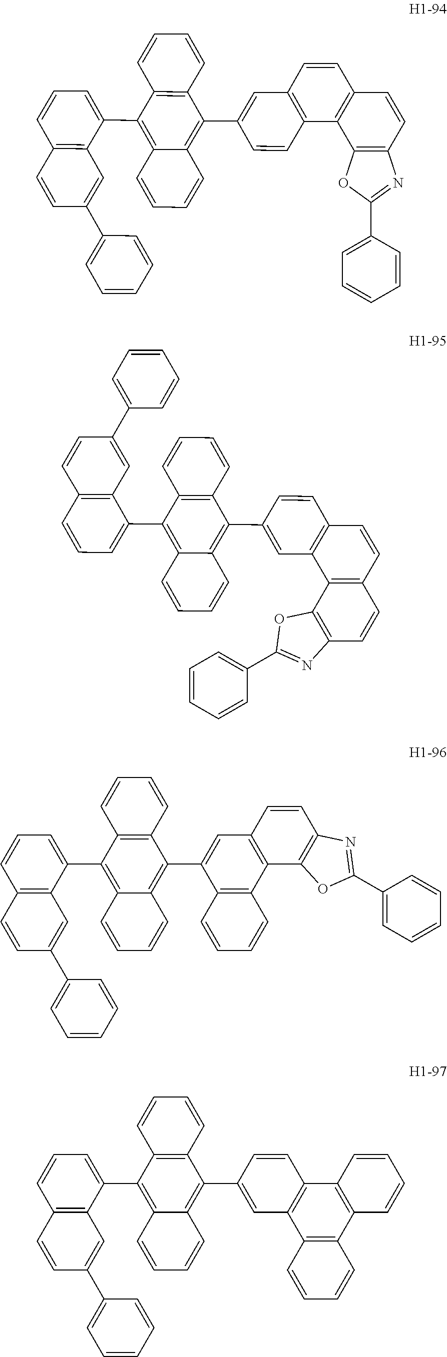

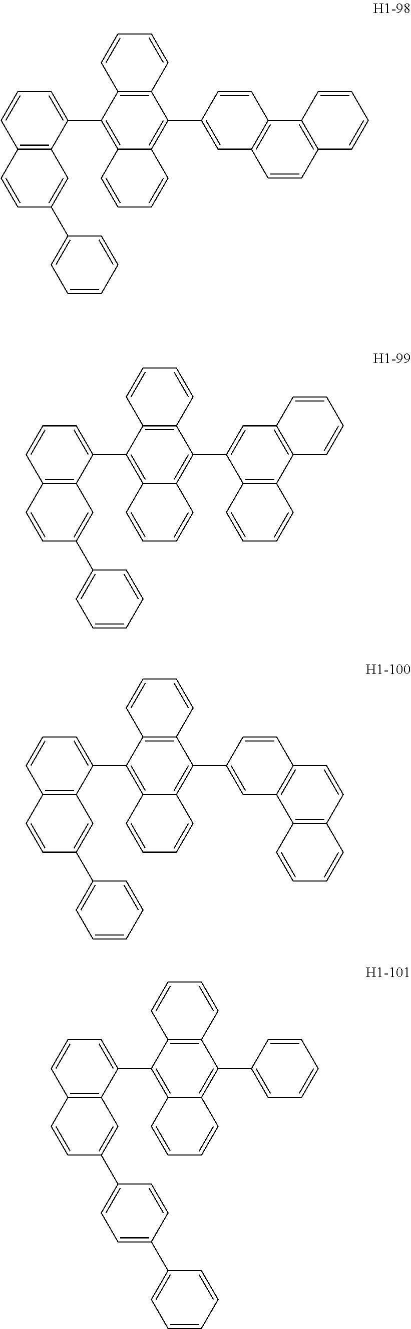

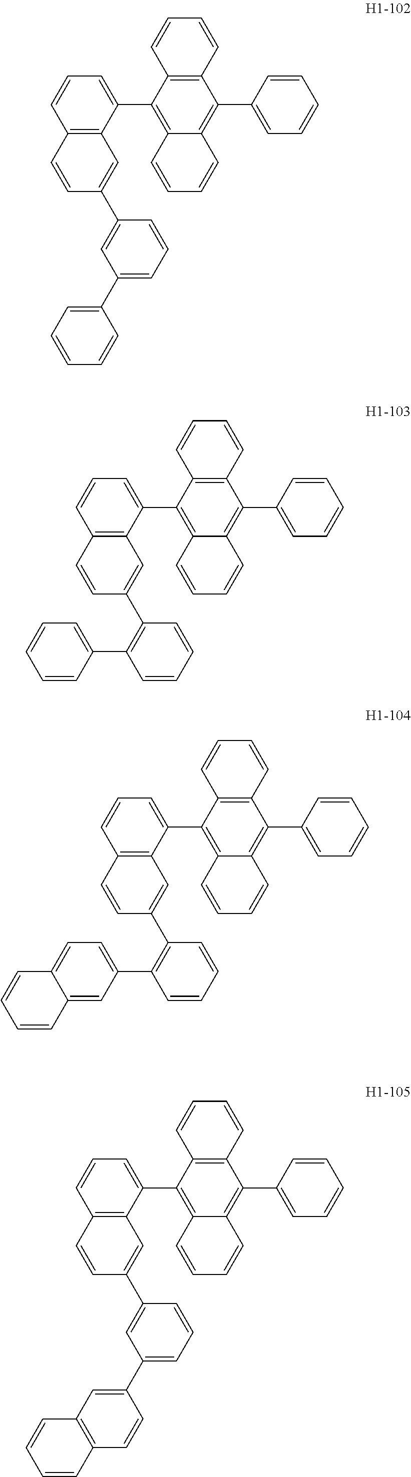

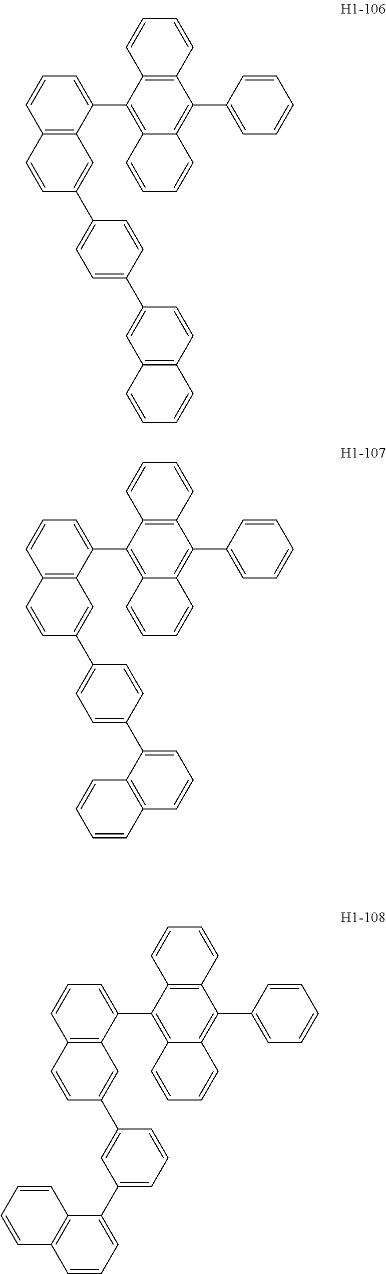

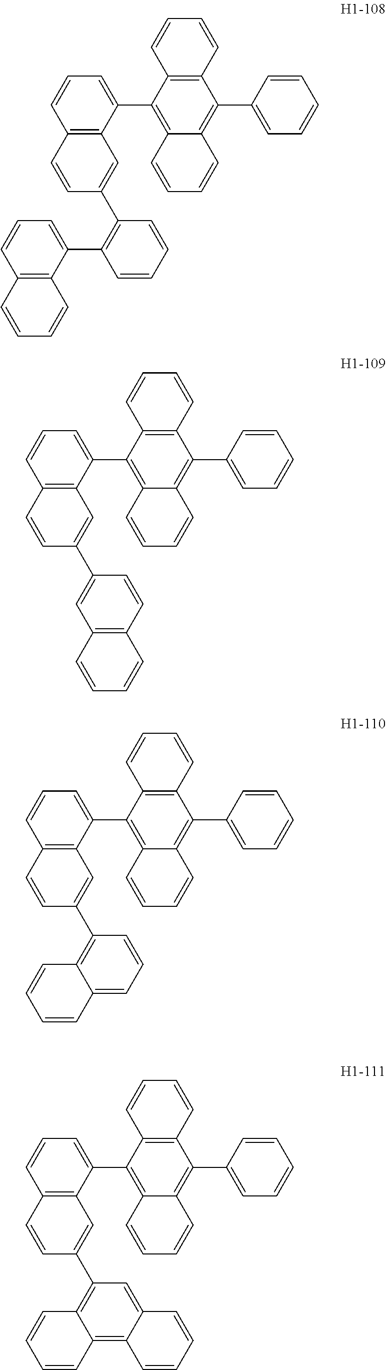

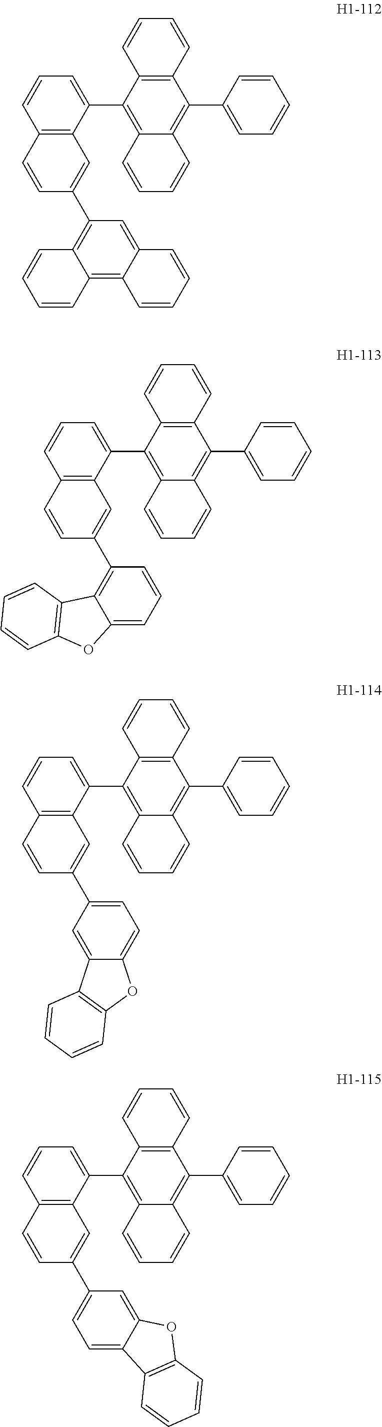

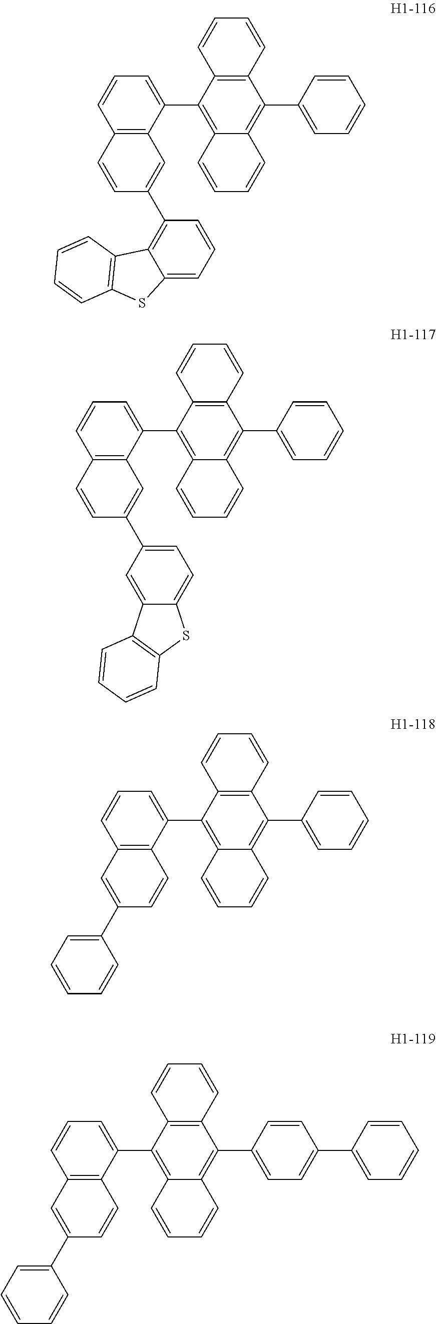

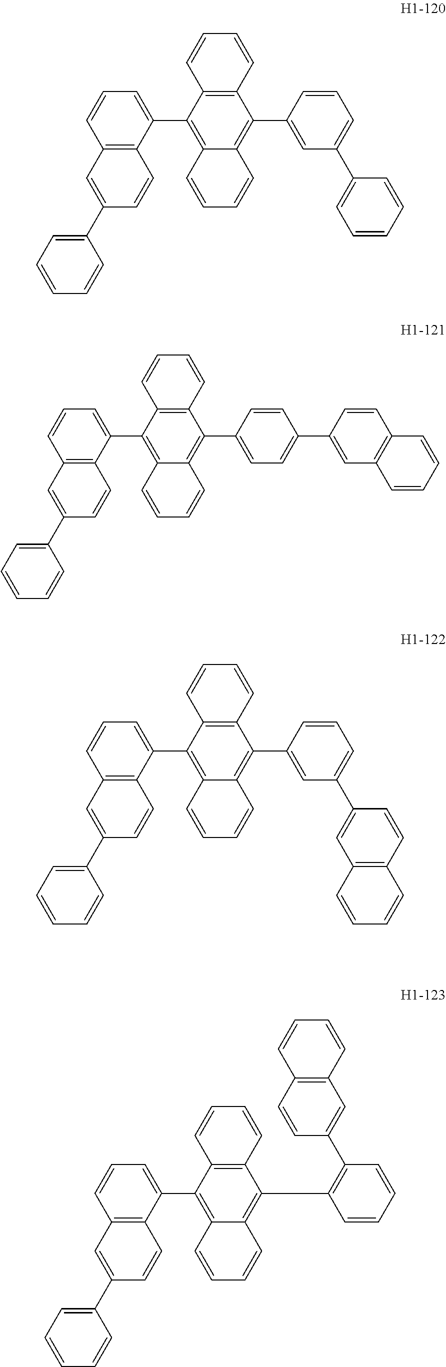

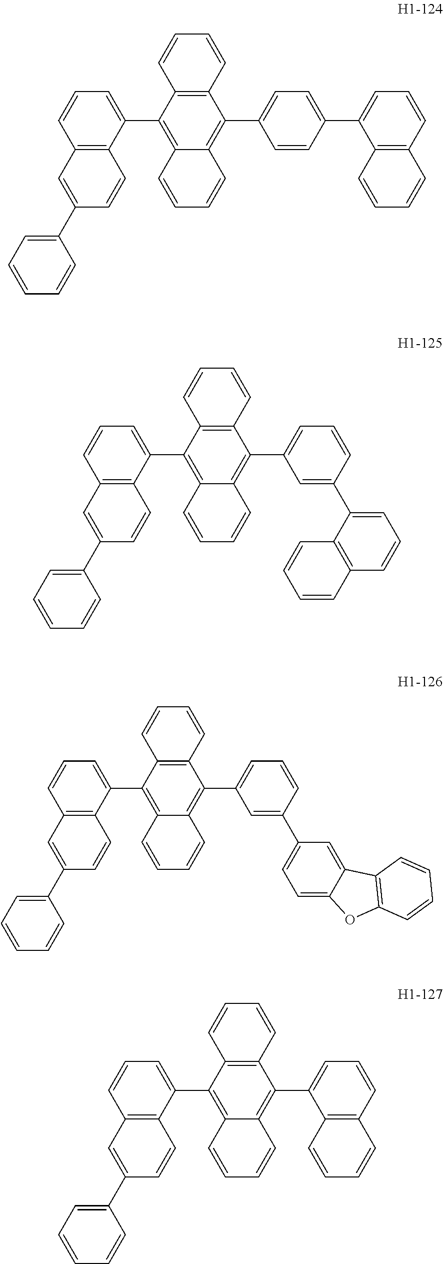

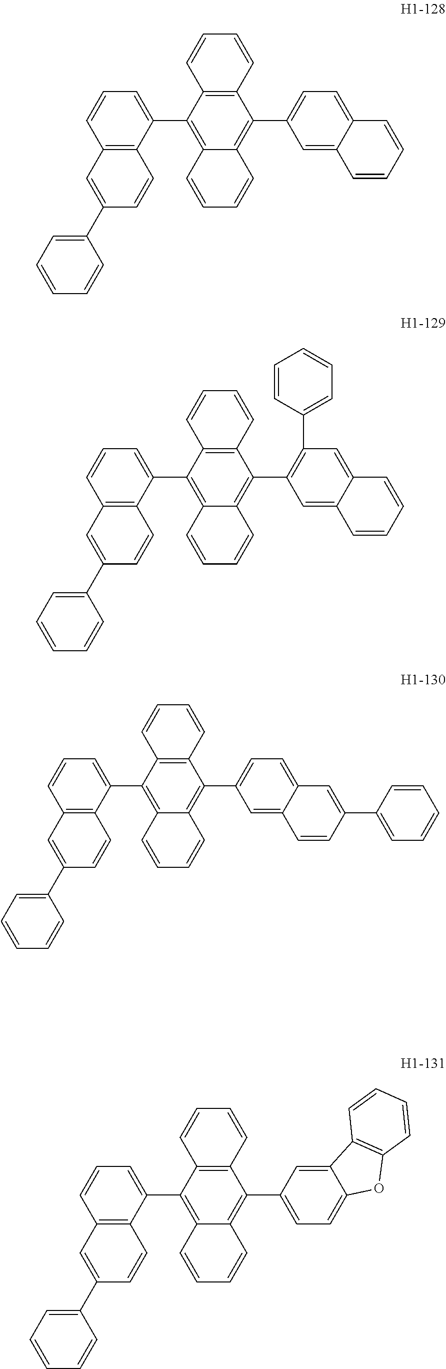

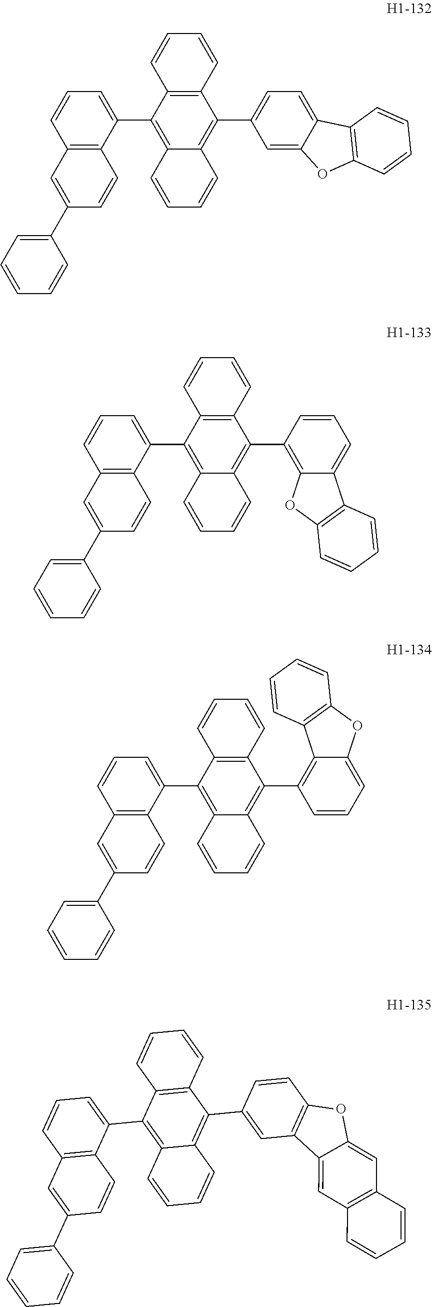 [SMILES: c1ccc(-c2ccc3c(-c4c5ccccc5c(-c5ccc6c(c5)oc5ccccc56)c5ccccc45)cccc3c2)cc1 ; c1ccc(-c2ccc3c(-c4c5ccccc5c(-c5cccc6c5oc5ccccc56)c5ccccc45)cccc3c2)cc1 ; c1ccc(-c2ccc3c(-c4c5ccccc5c(-c5cccc6oc7ccccc7c56)c5ccccc45)cccc3c2)cc1 ; c1ccc(-c2ccc3c(-c4c5ccccc5c(-c5ccc6oc7cc8ccccc8cc7c6c5)c5ccccc45)cccc3c2)cc1]

The light-emitting material according to one embodiment comprises at least one boron derivative represented by formula 2. For example, the compound represented by formula 2 may be a fluorescent dopant, e.g., a fluorescent blue dopant.

In formula 2, ring A, ring B, and ring C, each independently, represent a substituted or unsubstituted (C6-C30)aryl, or a substituted or unsubstituted (3- to 50-membered)heteroaryl, wherein ring B and ring C may be linked to each other to form a ring. According to one embodiment of the present disclosure, ring A, ring B, and ring C, each independently, represent a substituted or unsubstituted (C6-C25)aryl, or a substituted or unsubstituted (5- to 40-membered)heteroaryl, wherein ring B and ring C may be linked to each other to form a ring. According to another embodiment of the present disclosure, ring A represents a substituted or unsubstituted (C6-C18)aryl; and ring B and ring C, each independently, represent a substituted or unsubstituted (C6-C18)aryl, or a substituted or unsubstituted (5- to 36-membered)heteroaryl. For example, ring A may represent a substituted or unsubstituted benzene ring, an unsubstituted naphthalene ring, or an unsubstituted terphenyl ring. The substituents of the substituted benzene ring may be at least one selected from the group consisting of deuterium; a methyl unsubstituted or substituted with at least one deuterium; a tert-butyl; a diphenylamino unsubstituted or substituted with at least one of deuterium, a methyl(s) and a tert-butyl(s); a phenylnaphthylamino; a phenylbiphenylamino unsubstituted or substituted with tert-butyl(s); a dinaphthylamino; a dibiphenylamino; a phenyldibenzofuranylamino; a substituted or unsubstituted phenyl; a biphenyl; a terphenyl; a triphenylenyl; a carbazolyl; a phenoxazinyl; a phenothiazinyl; a dimethylacridinyl; and a dimethylxantenyl, in which the substituents of the substituted phenyl may be at least one of a methyl(s), a carbazolyl(s), a dibenzofuranyl(s), a diphenylamino(s), a phenoxazinyl(s), a phenothiazinyl(s), and a dimethylacridinyl(s). For example, ring B and ring C, each independently, may represent a substituted or unsubstituted benzene ring, an unsubstituted naphthalene ring, an unsubstituted dibenzothiophene ring, an unsubstituted dibenzofuran ring, a carbazole ring substituted with at least one of a phenyl(s) and a diphenylamino(s), a boron- and nitrogen-containing (21-membered)hetero ring substituted with at least one of a methyl(s) and a phenyl(s), a boron- and nitrogen-containing (25-membered)hetero ring substituted with at least one phenyl(s), or a boron- and nitrogen-containing (36-membered)hetero ring substituted with at least one methyl(s). The substituents of the substituted benzene ring may be at least one selected from the group consisting of a methyl, tert-butyl, a phenyl, a naphthyl, a substituted or unsubstituted diphenylamino, a phenylnaphthylamino, and a phenylamino substituted with a phenylcarbazolyl(s) or a dibenzofuranyl(s). The substituents of the substituted diphenylamino may be at least one of a methyl(s) and/or a diphenylamino(s).

In formula 2, Y₁ represents B; and X₁ and X₂, each independently, represent NR or O. R each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to at least one of ring A, ring B, and ring C to form a ring(s). According to one embodiment of the present disclosure, R each independently represents hydrogen, deuterium, a substituted or unsubstituted (C1-C20)alkyl, a substituted or unsubstituted (C6-C25)aryl, or a substituted or unsubstituted (3- to 25-membered)heteroaryl; or may be linked to at least one of ring A, ring B, and ring C to form a ring(s). According to another embodiment of the present disclosure, R each independently represents hydrogen, deuterium, an unsubstituted (C1-C10)alkyl, a (C6-C18)aryl unsubstituted or substituted with at least one of a (C1-C10)alkyl(s) and a di(C6-C18)arylamino(s), or a (5- to 20-membered)heteroaryl substituted with a (C6-C18)aryl(s); or may be linked to at least one of ring A, ring B, and ring C to form a ring(s). For example, R each independently may represent hydrogen, deuterium, a methyl, a phenyl unsubstituted or substituted with at least one of a methyl(s) and/or a tert-butyl(s), a naphthyl, a biphenyl unsubstituted or substituted with a diphenylamino(s), a triphenylenyl, or a carbazolyl substituted with a phenyl(s), or may be linked to at least one of ring A, ring B, and ring C to form a ring(s).

According to one embodiment of the present disclosure, formula 2 may be represented by the following formula 2-1.

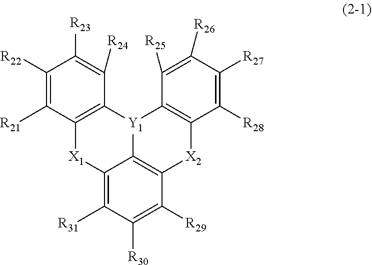

In formula 2-1,
Y₁, X₁, and X₂, each are as defined in formula 2; and
R₂₁ to R₃₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a ring(s). According to one embodiment of the present disclosure, R₂₁ to R₃₁, each independently, represent hydrogen, deuterium, a substituted or unsubstituted (C1-C20)alkyl, a substituted or unsubstituted (C6-C25)aryl, a substituted or unsubstituted (5- to 20-membered)heteroaryl, or a substituted or unsubstituted mono- or di-(C6-C25)arylamino; or may be linked to an adjacent substituent to form a ring(s). According to another embodiment of the present disclosure, R₂₁ to R₃₁, each independently, represent hydrogen, deuterium, a (C1-C10)alkyl unsubstituted or substituted with at least one deuterium; a (C6-C18)aryl unsubstituted or substituted with at least one of deuterium, a (C1-C10)alkyl(s), a (13- to 18-membered)heteroaryl(s), and a di(C6)arylamino(s); a (5- to 18-membered)heteroaryl unsubstituted or substituted with at least one of deuterium and a (C1-C10)alkyl(s); or a mono- or di-(C6-C18)arylamino unsubstituted or substituted with at least one of deuterium, a (C1-C10)alkyl(s), a di(C6-C18)arylamino(s), and a (13- to 20-membered)heteroaryl(s); or may be linked to an adjacent substituent to form a ring(s). For example, R₂₁ to R₃₁, each independently, may represent hydrogen, a methyl, a tert-butyl, a substituted or unsubstituted phenyl, a naphthyl, a biphenyl, a terphenyl, a triphenylenyl, a carbazolyl, a phenoxazinyl, a phenothiazinyl, a dimethylacridinyl, a dimethylxantenyl, a diphenylamino unsubstituted or substituted with at least one of a methyl(s) and a diphenylamino(s), a phenylnaphthylamino, a phenylbiphenylamino unsubstituted or substituted with a tert-butyl(s), a dinaphthylamino, a dibiphenylamino, a phenylamino substituted with a phenylcarbazolyl(s) or a dibenzofuranyl(s), or a (17- to 21-membered)heteroaryl substituted with at least one of a methyl(s) and a phenyl(s); or may be linked to an adjacent substituent to form a benzene ring(s), an indole ring(s) substituted with a phenyl(s) and/or a diphenylamino(s), a benzofuran ring(s), a benzothiophene ring(s), or a (19-membered)hetero ring(s) substituted with at least one methyl(s). In addition, R₂₄ and R₂₅ may be linked to each other via —O—. The substituents of the substituted phenyl may be at least one of a methyl(s), a carbazolyl(s), a dibenzofuranyl(s), a diphenylamino(s), a phenoxazinyl(s), a phenothiazinyl(s) and a dimethylacridinyl(s).

The compound represented by formula 2 may be at least one selected from the following compounds, but is not limited thereto.

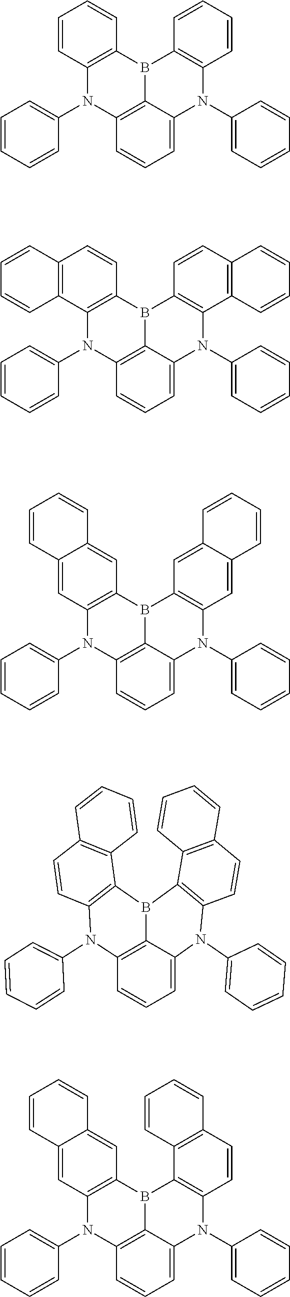 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1]

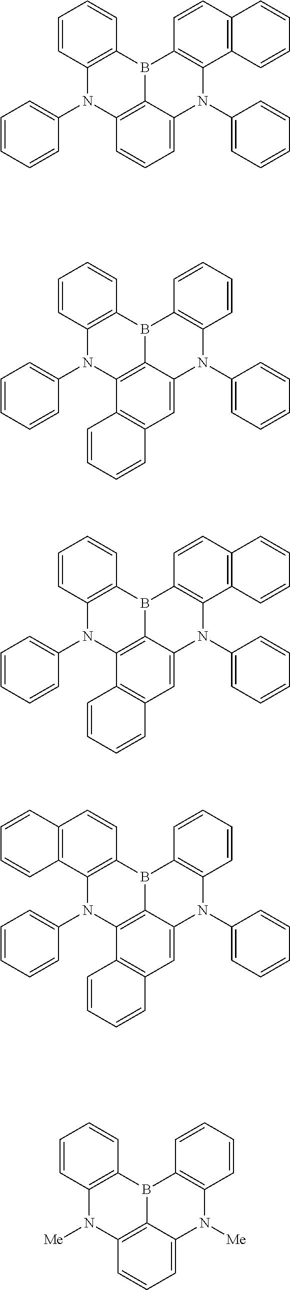 [SMILES: c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32]

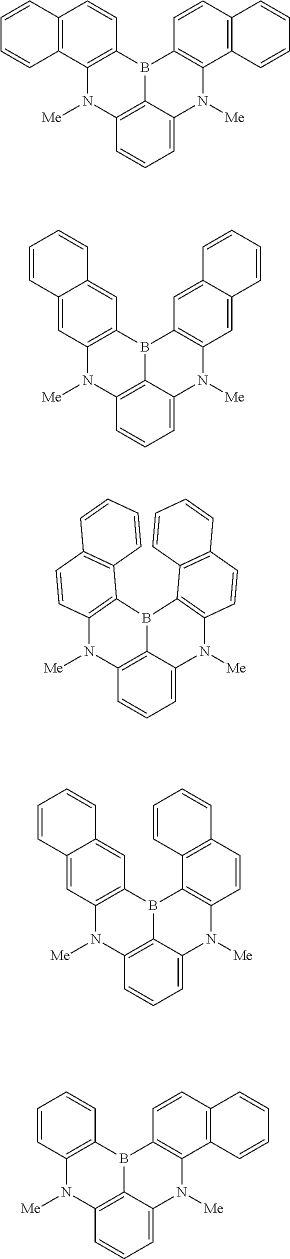 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

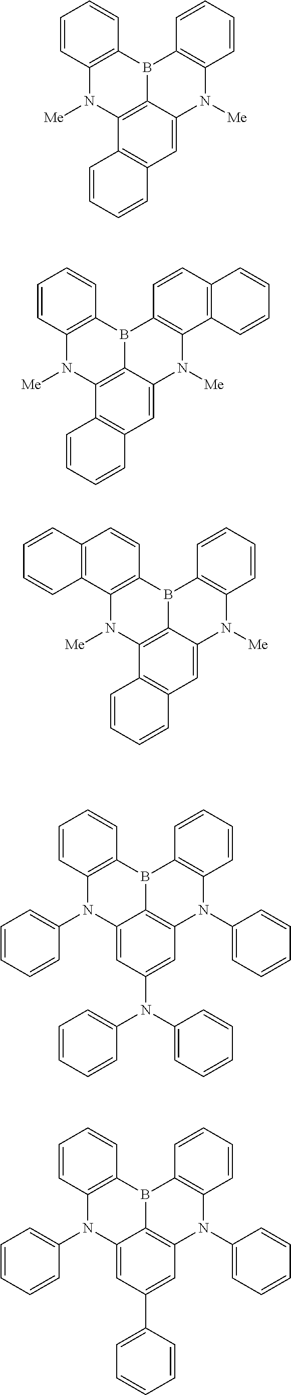

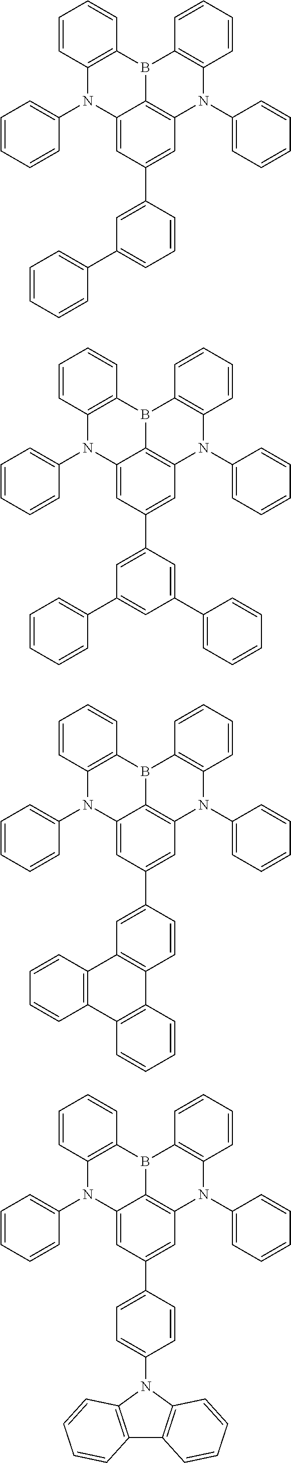

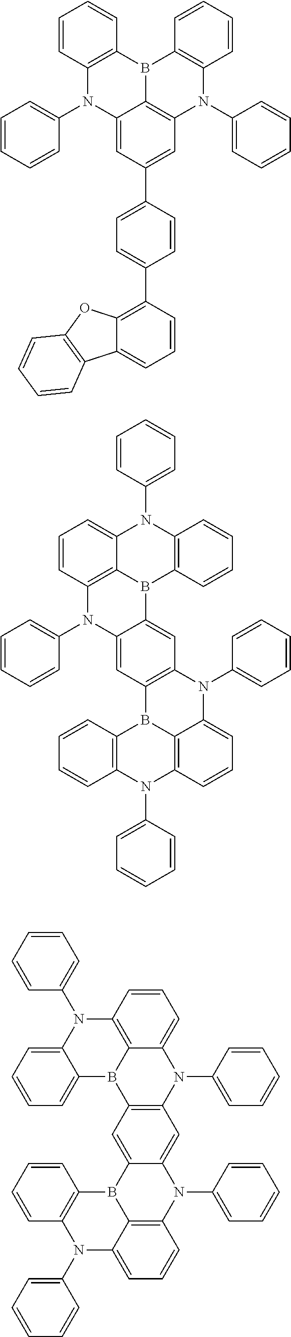 [SMILES: c1ccc(N2c3ccccc3B3c4ccccc4N(c4ccccc4)c4cc(-c5ccc(-c6cccc7c6oc6ccccc67)cc5)cc2c43)cc1 ; c1ccc(N2c3ccccc3B3c4cc5c(cc4N(c4ccccc4)c4cccc2c43)B2c3ccccc3N(c3ccccc3)c3cccc(c32)N5c2ccccc2)cc1 ; c1ccc(N2c3ccccc3B3c4cc5c(cc4N(c4ccccc4)c4cccc2c43)N(c2ccccc2)c2cccc3c2B5c2ccccc2N3c2ccccc2)cc1]

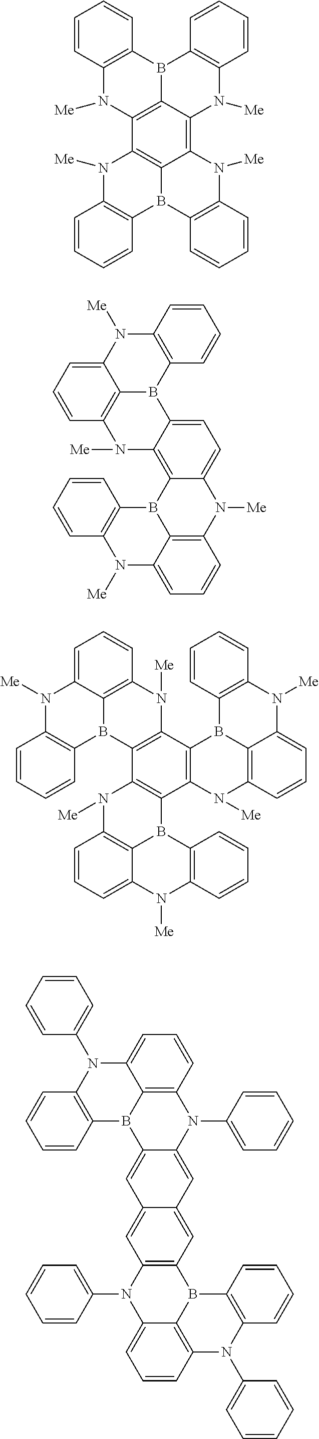

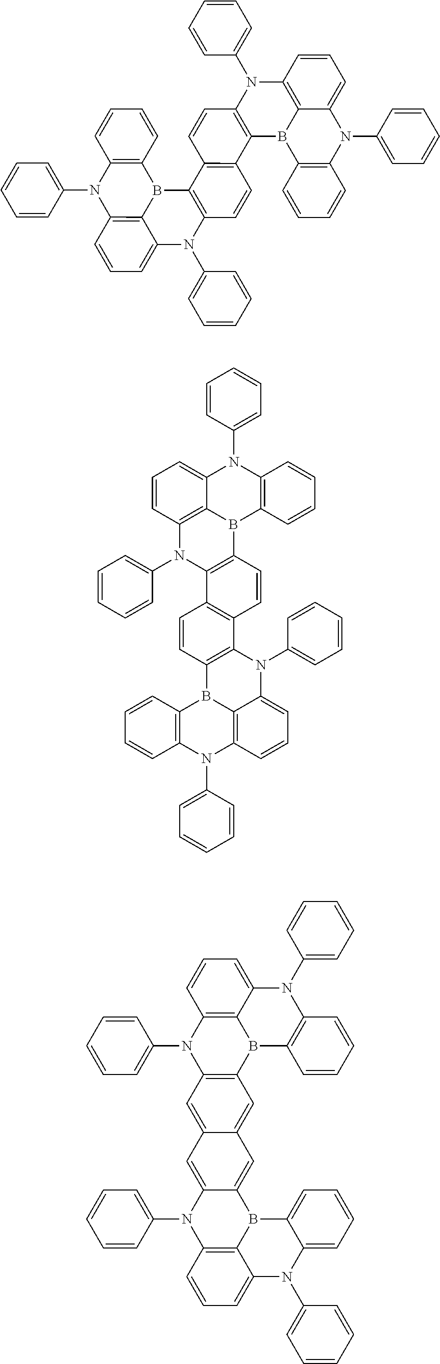

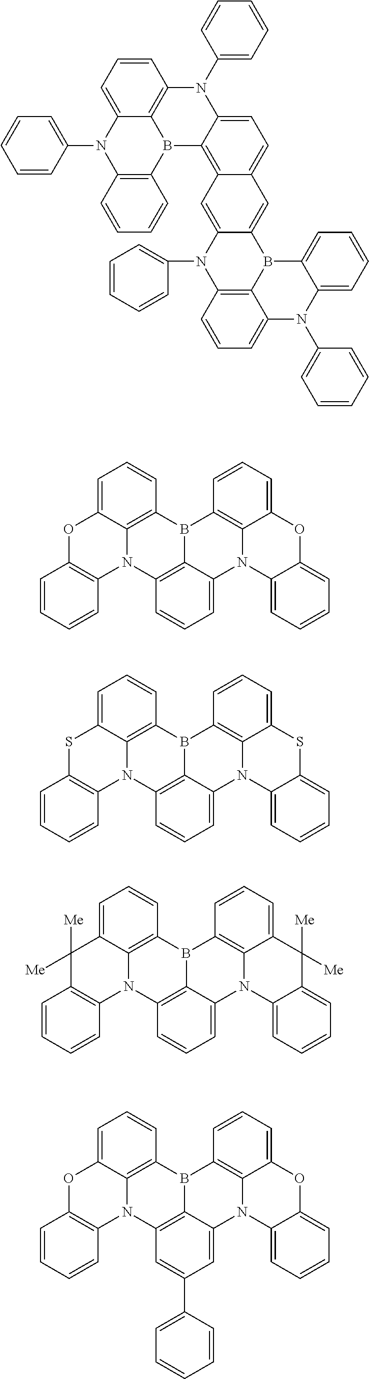 [SMILES: c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1 ; c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C ; c1ccc(-c2cc3c4c(c2)N2c5ccccc5Oc5cccc(c52)B4c2cccc4c2N3c2ccccc2O4)cc1]

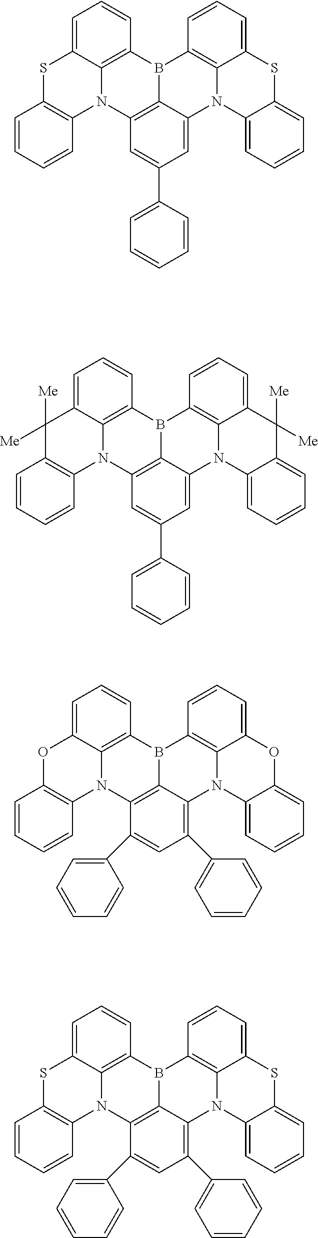 [SMILES: c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccccc4)cc4c3B(c3cccc1c32)c1cccc2c1N4c1ccccc1C2(C)C ; c1ccc(-c2cc(-c3ccccc3)c3c4c2N2c5ccccc5Oc5cccc(c52)B4c2cccc4c2N3c2ccccc2O4)cc1 ; c1ccc(-c2cc(-c3ccccc3)c3c4c2N2c5ccccc5Sc5cccc(c52)B4c2cccc4c2N3c2ccccc2S4)cc1]

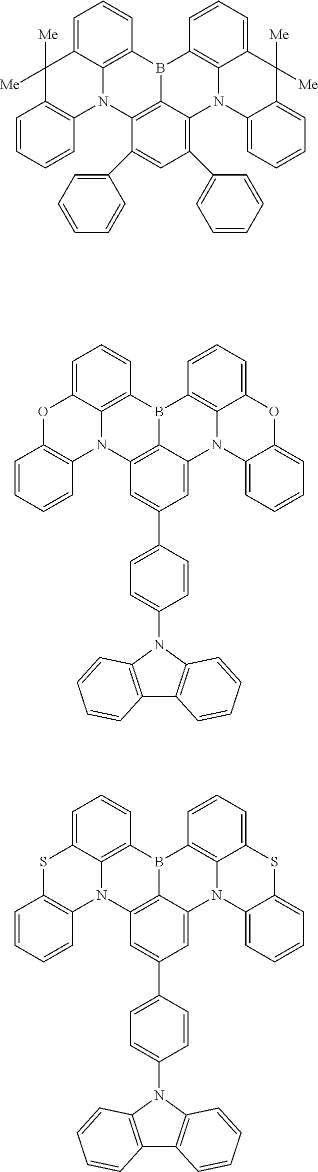 [SMILES: CC1(C)c2ccccc2N2c3c(cccc31)B1c3cccc4c3N(c3ccccc3C4(C)C)c3c(-c4ccccc4)cc(-c4ccccc4)c2c31 ; c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3]

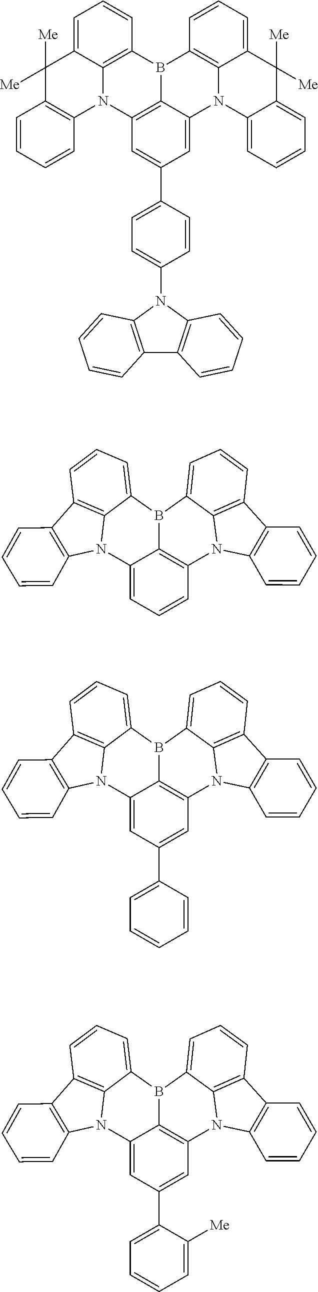 [SMILES: CC1(C)c2ccccc2N2c3cc(-c4ccc(-n5c6ccccc6c6ccccc65)cc4)cc4c3B(c3cccc1c32)c1cccc2c1N4c1ccccc1C2(C)C ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

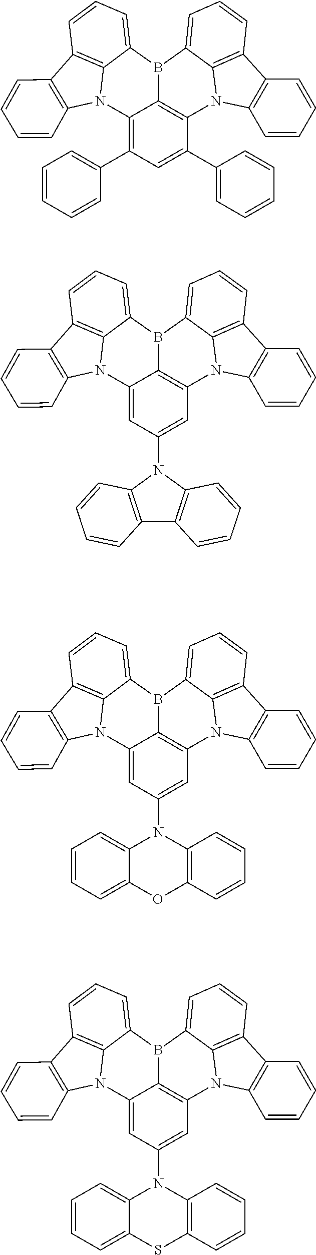 [SMILES: c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13]

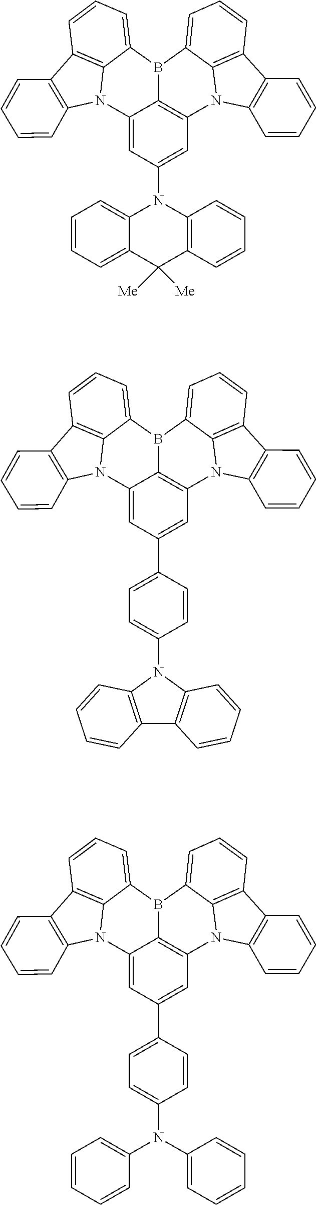 [SMILES: CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1]

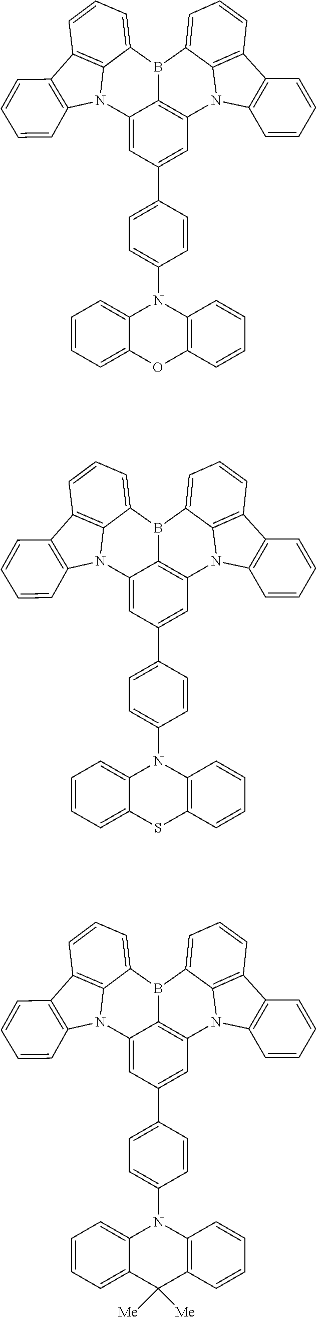 [SMILES: c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

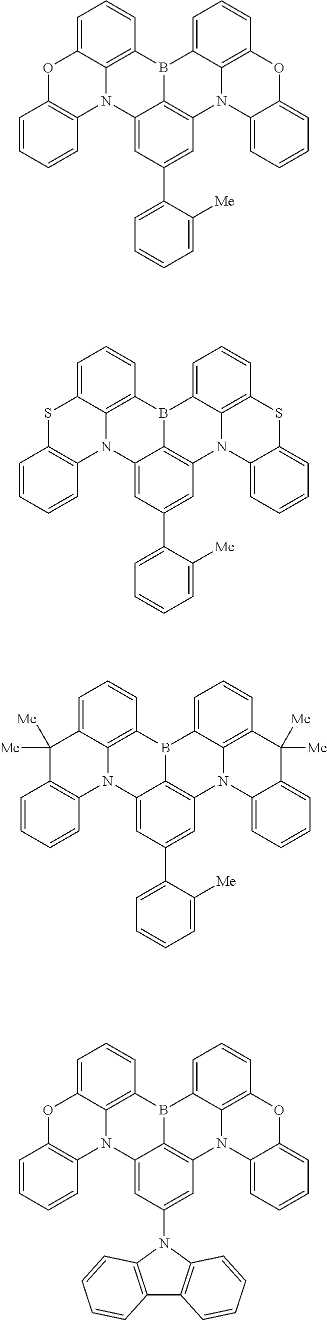 [SMILES: Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C ; c1ccc2c(c1)Oc1cccc3c1N2c1cc(-n2c4ccccc4c4ccccc42)cc2c1B3c1cccc3c1N2c1ccccc1O3]

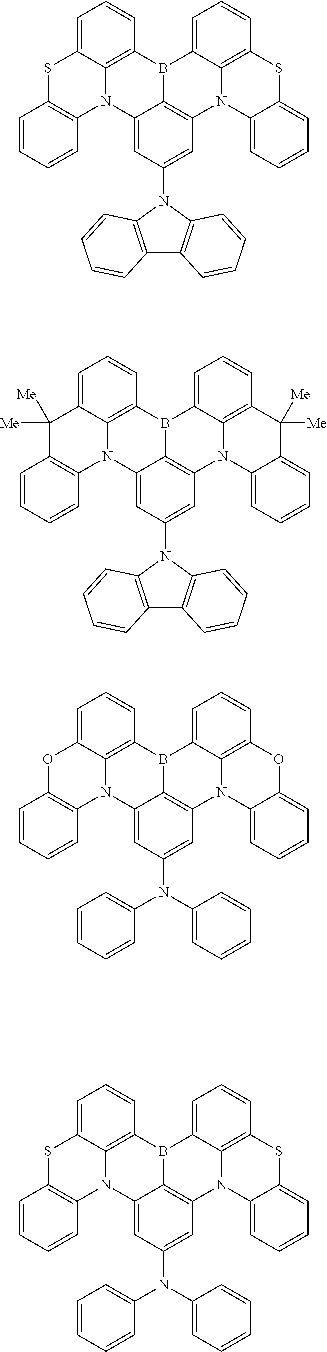 [SMILES: c1ccc2c(c1)Sc1cccc3c1N2c1cc(-n2c4ccccc4c4ccccc42)cc2c1B3c1cccc3c1N2c1ccccc1S3 ; CC1(C)c2ccccc2N2c3cc(-n4c5ccccc5c5ccccc54)cc4c3B(c3cccc1c32)c1cccc2c1N4c1ccccc1C2(C)C ; c1ccc(N(c2ccccc2)c2cc3c4c(c2)N2c5ccccc5Oc5cccc(c52)B4c2cccc4c2N3c2ccccc2O4)cc1 ; c1ccc(N(c2ccccc2)c2cc3c4c(c2)N2c5ccccc5Sc5cccc(c52)B4c2cccc4c2N3c2ccccc2S4)cc1]

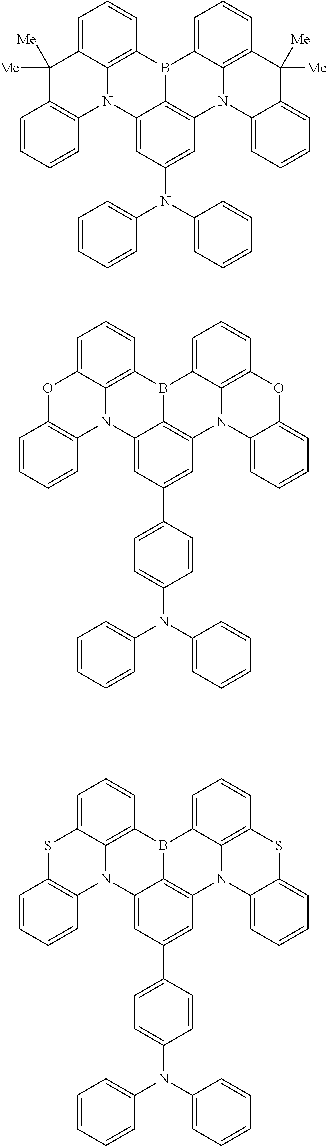 [SMILES: CC1(C)c2ccccc2N2c3cc(N(c4ccccc4)c4ccccc4)cc4c3B(c3cccc1c32)c1cccc2c1N4c1ccccc1C2(C)C ; c1ccc(N(c2ccccc2)c2ccc(-c3cc4c5c(c3)N3c6ccccc6Oc6cccc(c63)B5c3cccc5c3N4c3ccccc3O5)cc2)cc1 ; c1ccc(N(c2ccccc2)c2ccc(-c3cc4c5c(c3)N3c6ccccc6Sc6cccc(c63)B5c3cccc5c3N4c3ccccc3S5)cc2)cc1]

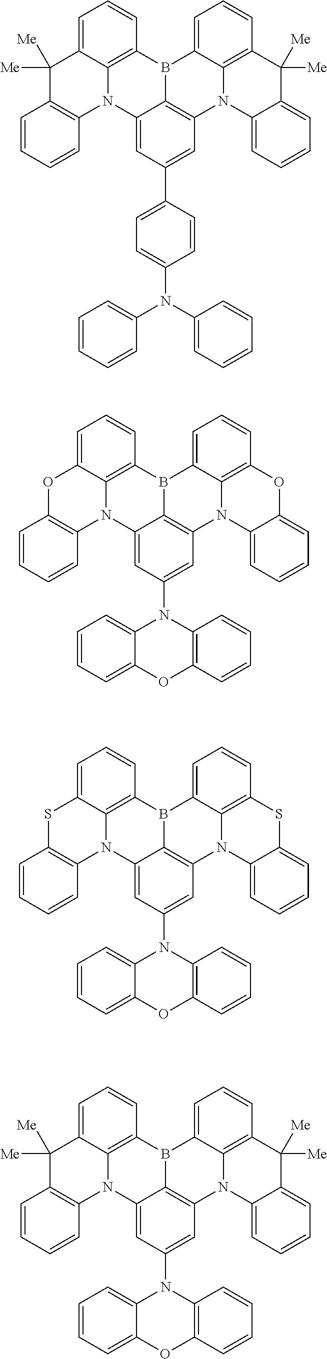 [SMILES: CC1(C)c2ccccc2N2c3cc(-c4ccc(N(c5ccccc5)c5ccccc5)cc4)cc4c3B(c3cccc1c32)c1cccc2c1N4c1ccccc1C2(C)C ; c1ccc2c(c1)Oc1ccccc1N2c1cc2c3c(c1)N1c4ccccc4Oc4cccc(c41)B3c1cccc3c1N2c1ccccc1O3 ; c1ccc2c(c1)Oc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Oc5ccccc54)cc4c3B(c3cccc1c32)c1cccc2c1N4c1ccccc1C2(C)C]

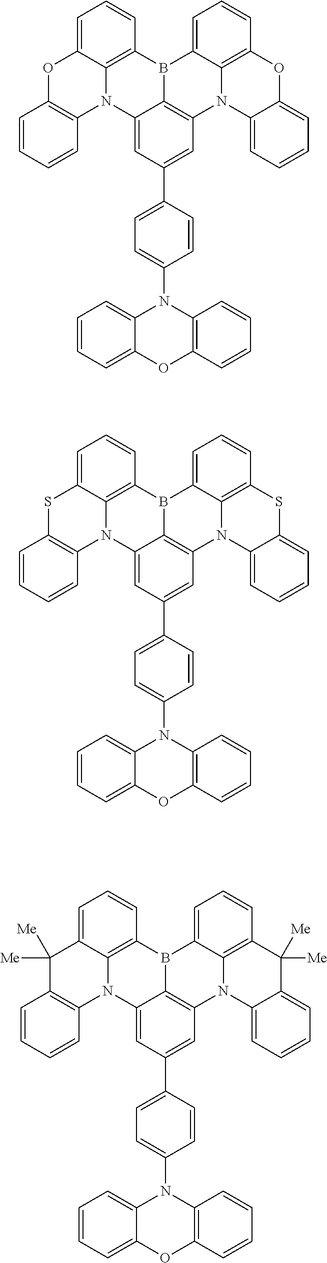 [SMILES: c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc2c(c1)Oc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Oc6ccccc65)cc4)cc4c3B(c3cccc1c32)c1cccc2c1N4c1ccccc1C2(C)C]

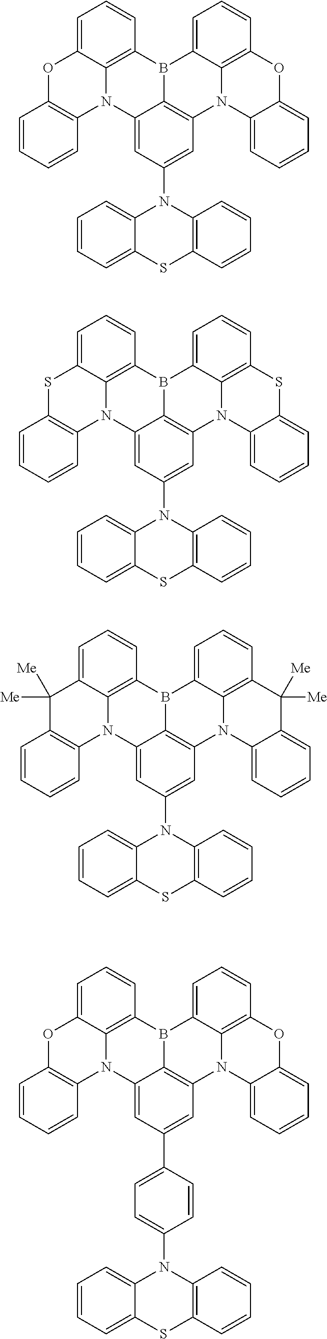 [SMILES: c1ccc2c(c1)Oc1cccc3c1N2c1cc(N2c4ccccc4Sc4ccccc42)cc2c1B3c1cccc3c1N2c1ccccc1O3 ; c1ccc2c(c1)Sc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Sc5ccccc54)cc4c3B(c3cccc1c32)c1cccc2c1N4c1ccccc1C2(C)C ; c1ccc2c(c1)Oc1cccc3c1N2c1cc(-c2ccc(N4c5ccccc5Sc5ccccc54)cc2)cc2c1B3c1cccc3c1N2c1ccccc1O3]

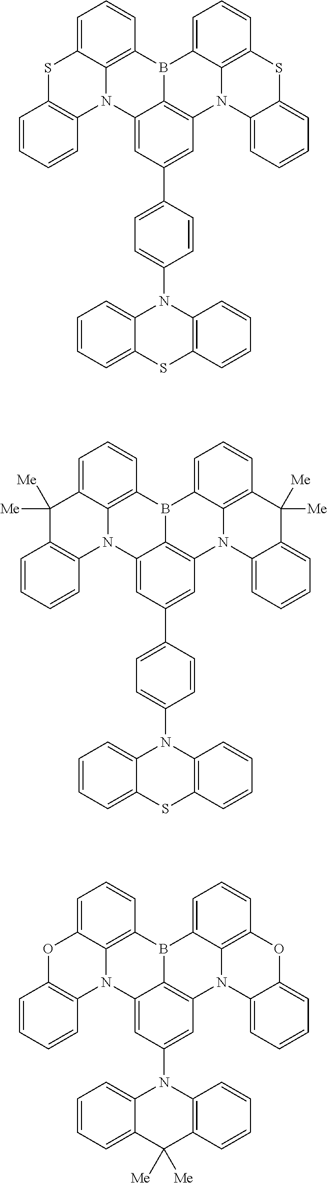 [SMILES: c1ccc2c(c1)Sc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Sc6ccccc65)cc4)cc4c3B(c3cccc1c32)c1cccc2c1N4c1ccccc1C2(C)C ; CC1(C)c2ccccc2N(c2cc3c4c(c2)N2c5ccccc5Oc5cccc(c52)B4c2cccc4c2N3c2ccccc2O4)c2ccccc21]

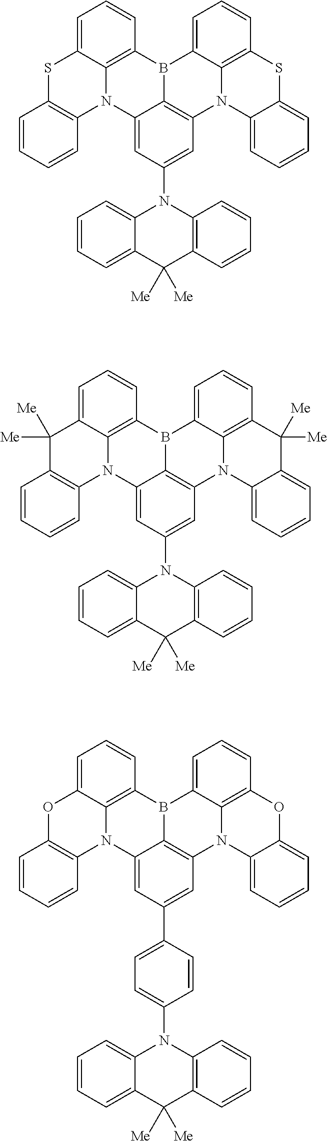 [SMILES: CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21]

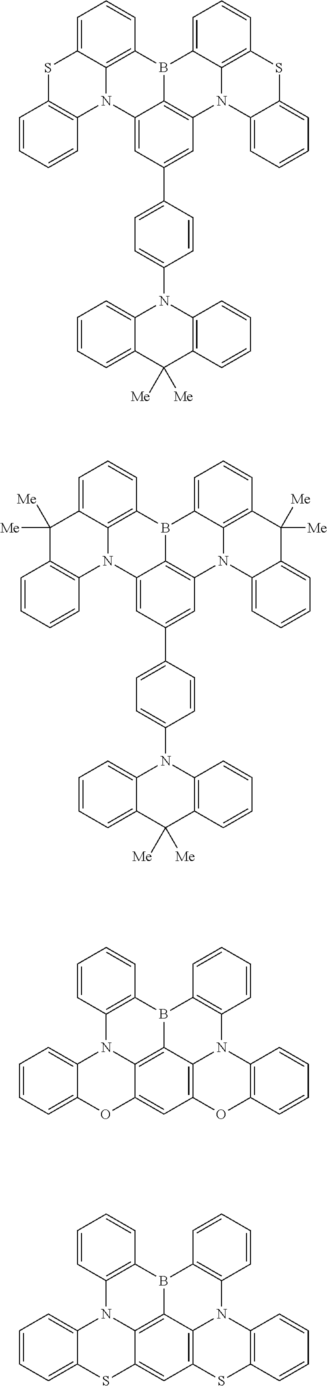 [SMILES: CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21 ; c1ccc2c(c1)B1c3ccccc3-n3c4ccccc4oc4cc5oc6ccccc6n-2c5c1c43 ; c1ccc2c(c1)B1c3ccccc3-n3c4ccccc4sc4cc5sc6ccccc6n-2c5c1c43]

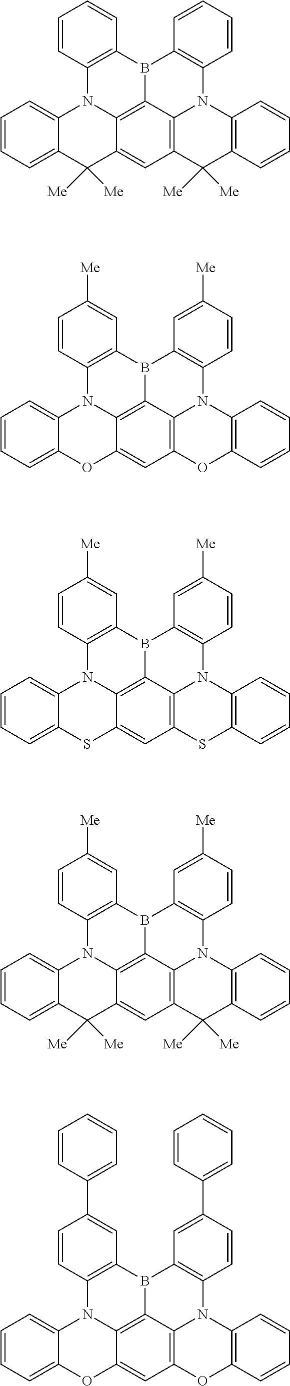 [SMILES: CC1(C)c2ccccc2N2c3ccccc3B3c4ccccc4N4c5ccccc5C(C)(C)c5cc1c2c3c54 ; Cc1ccc2c(c1)B1c3cc(C)ccc3-n3c4ccccc4oc4cc5oc6ccccc6n-2c5c1c43 ; Cc1ccc2c(c1)B1c3cc(C)ccc3-n3c4ccccc4sc4cc5sc6ccccc6n-2c5c1c43 ; Cc1ccc2c(c1)B1c3cc(C)ccc3N3c4ccccc4C(C)(C)c4cc5c(c1c43)N2c1ccccc1C5(C)C ; c1ccc(-c2ccc3c(c2)B2c4cc(-c5ccccc5)ccc4-n4c5ccccc5oc5cc6oc7ccccc7n-3c6c2c54)cc1]

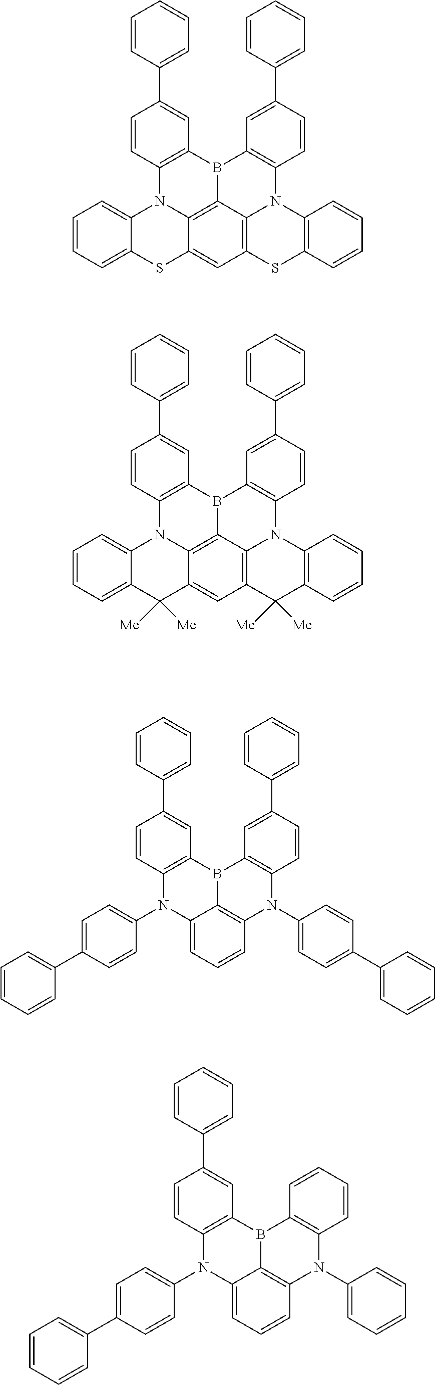 [SMILES: c1ccc(-c2ccc3c(c2)B2c4cc(-c5ccccc5)ccc4-n4c5ccccc5sc5cc6sc7ccccc7n-3c6c2c54)cc1 ; CC1(C)c2ccccc2N2c3ccc(-c4ccccc4)cc3B3c4cc(-c5ccccc5)ccc4N4c5ccccc5C(C)(C)c5cc1c2c3c54 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1]

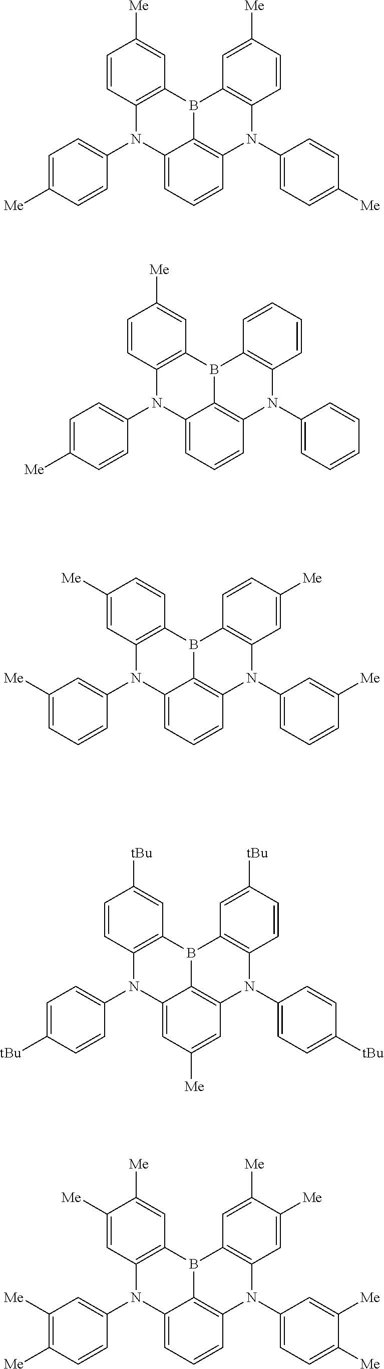 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; Cc1ccc(N2c3cc(C)c(C)cc3B3c4cc(C)c(C)cc4N(c4ccc(C)c(C)c4)c4cccc2c43)cc1C]

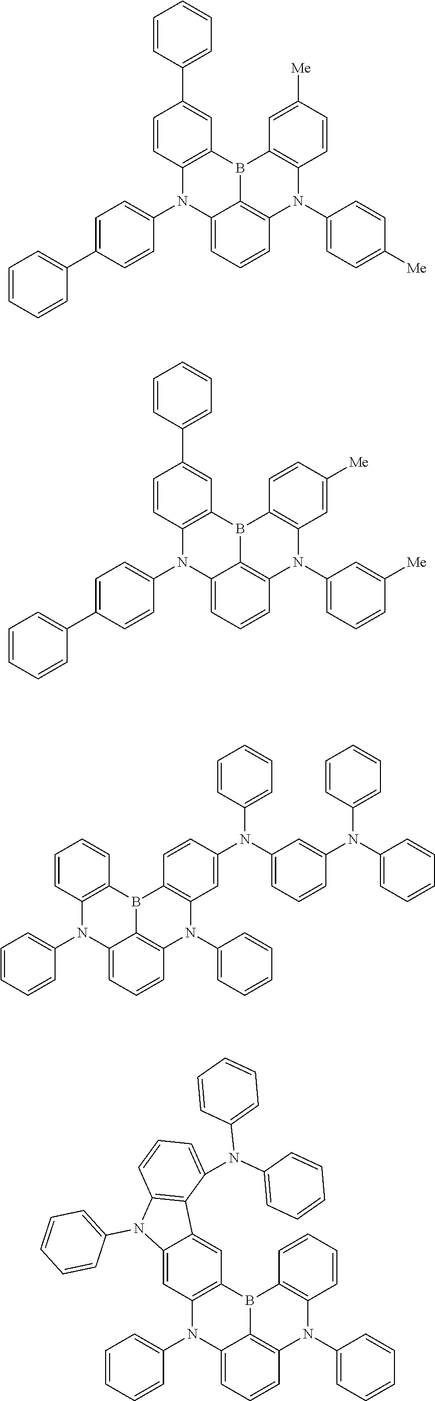 [SMILES: Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1]

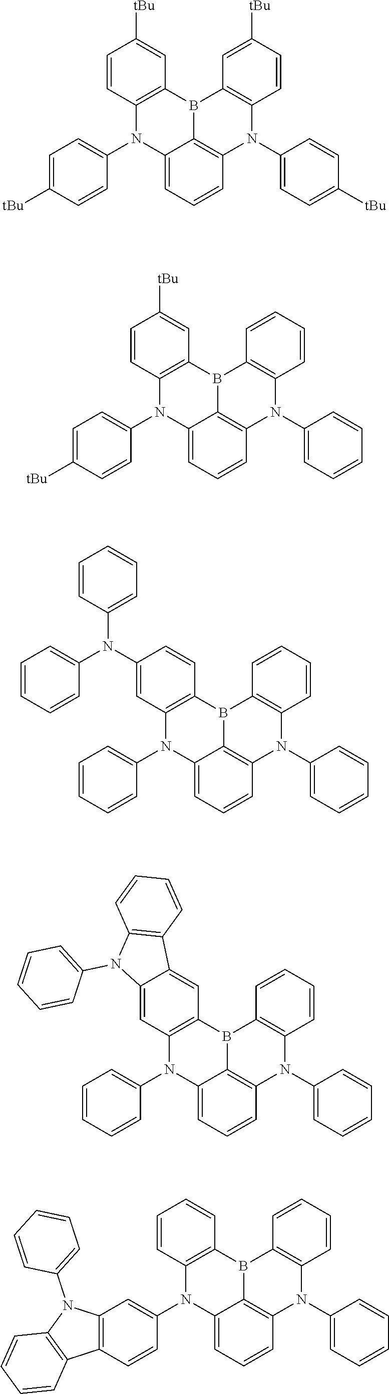 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1]

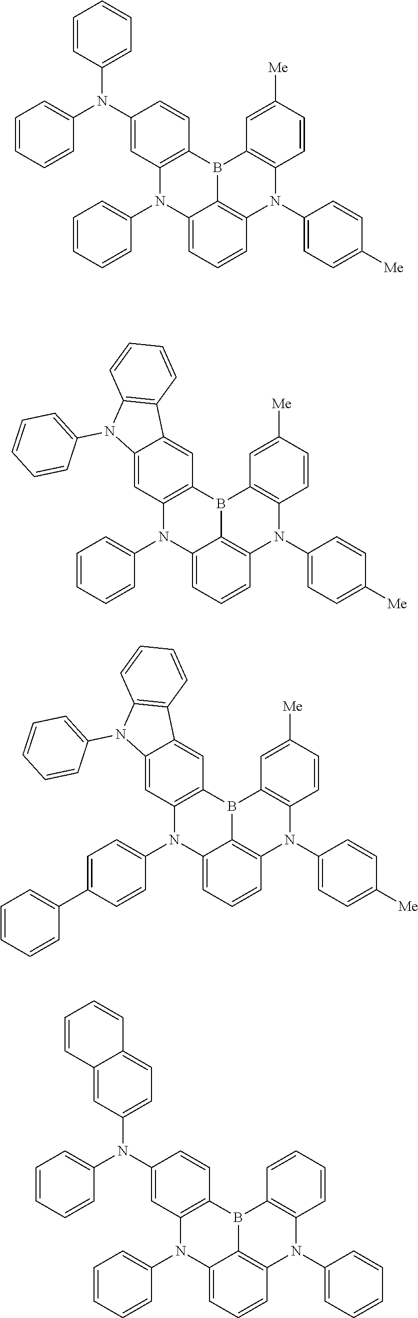 [SMILES: Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3ccccc3c2)cc1]

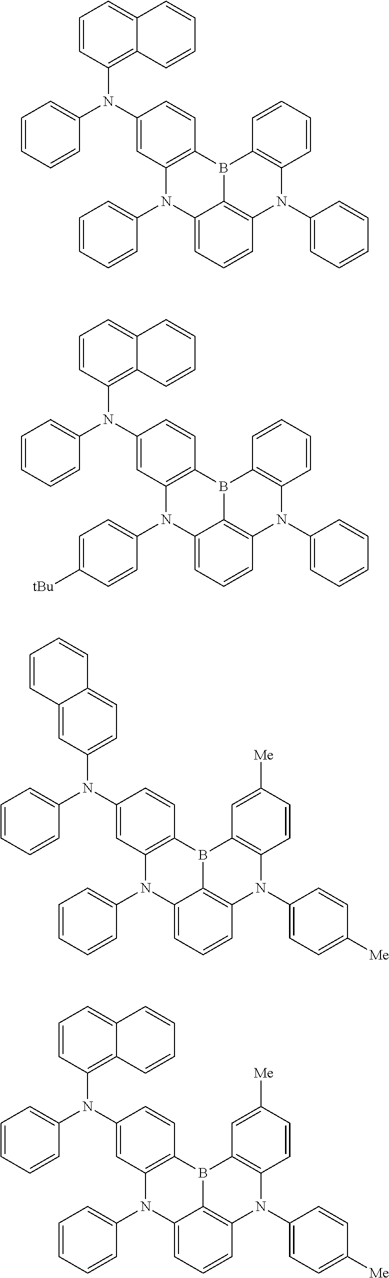 [SMILES: c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1]

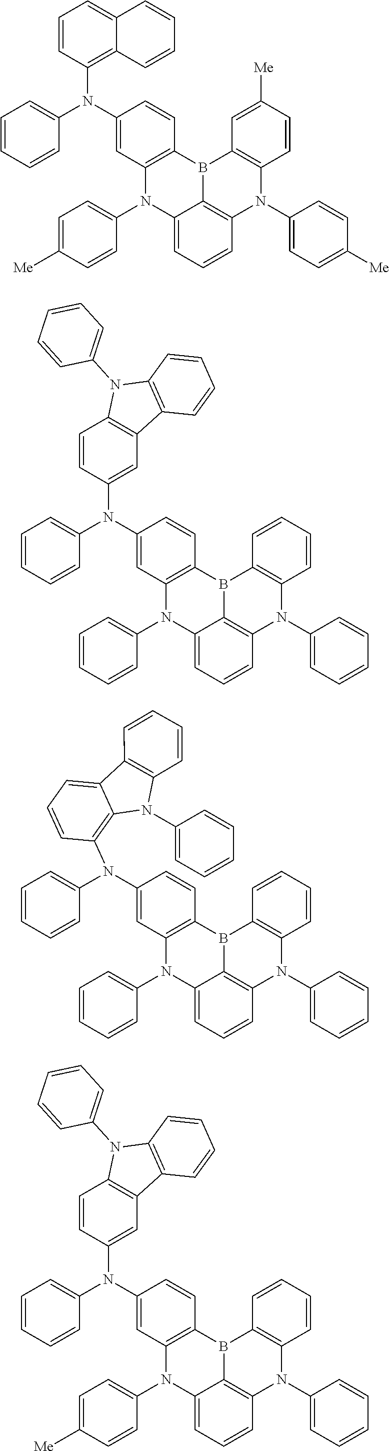

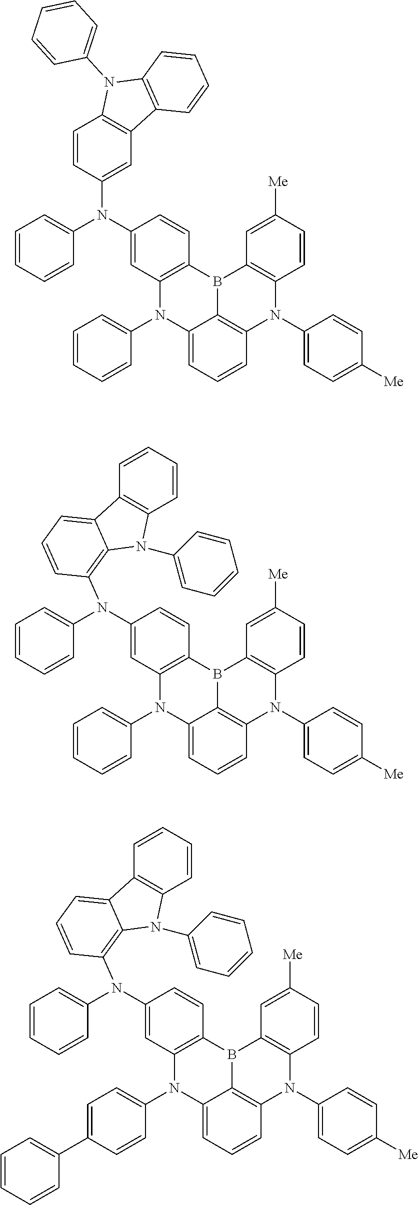 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

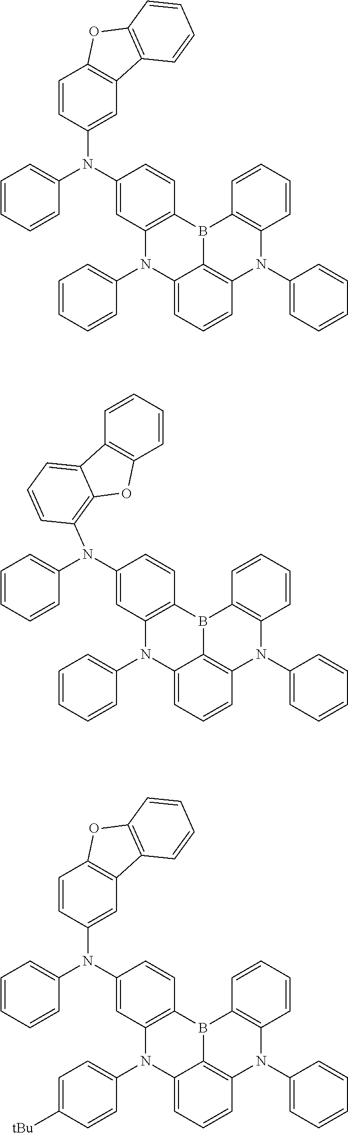 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

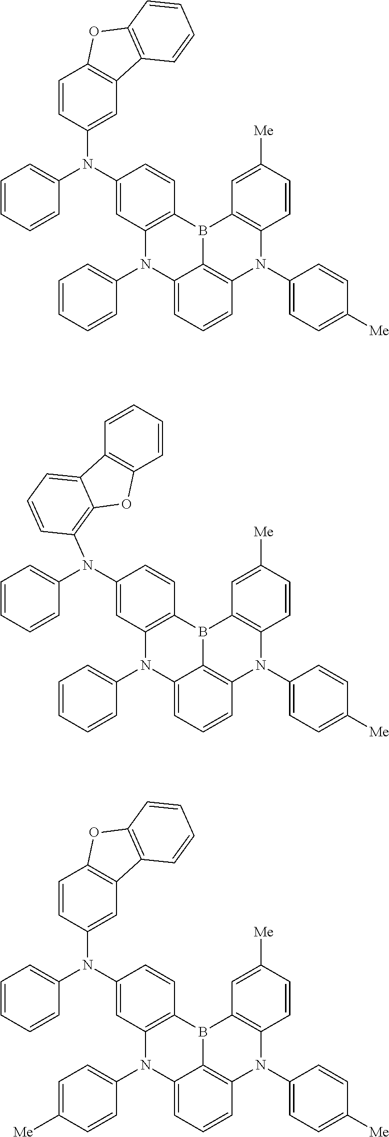 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

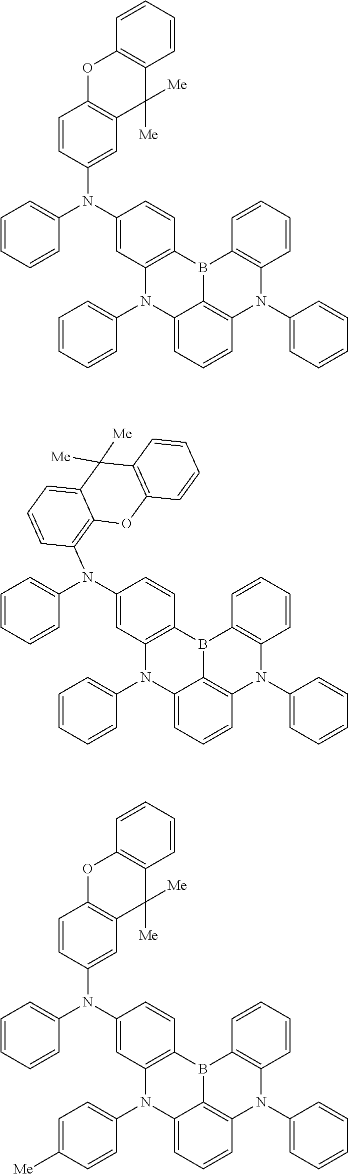 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

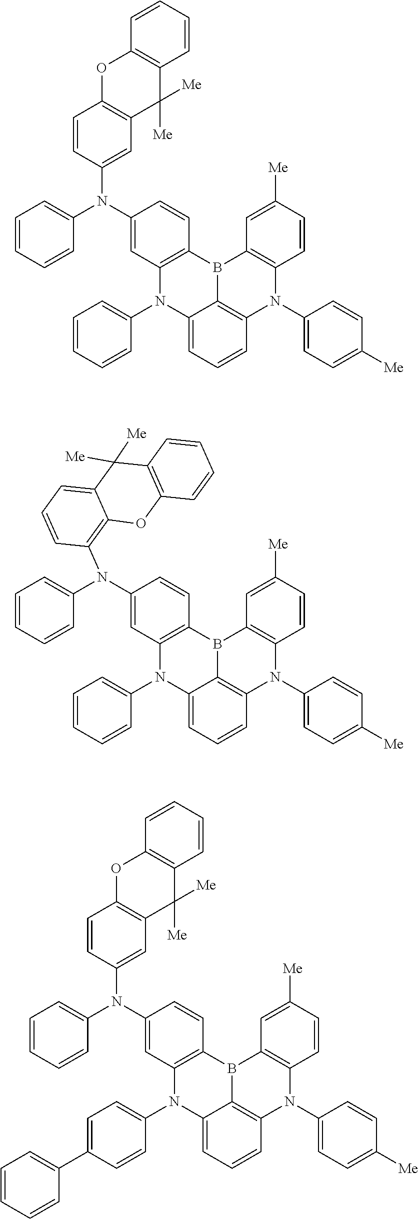 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

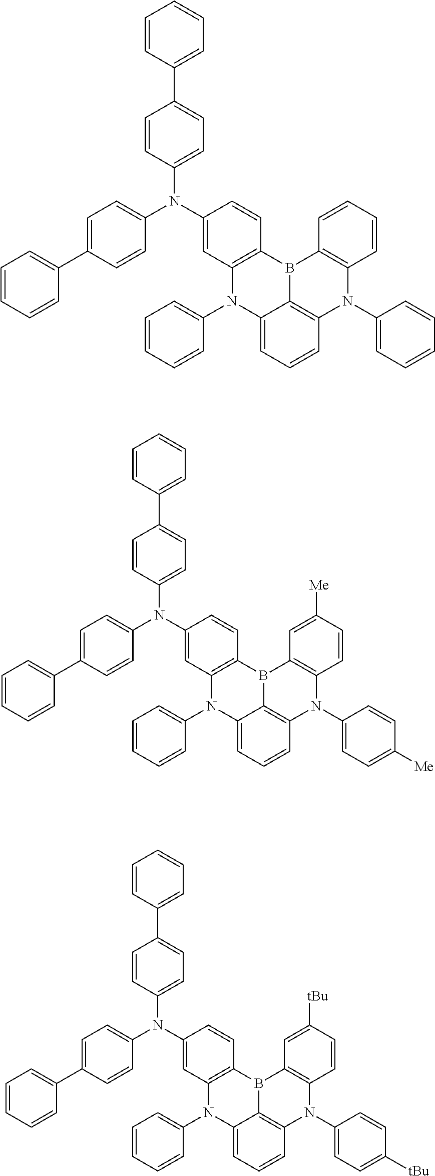 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1]

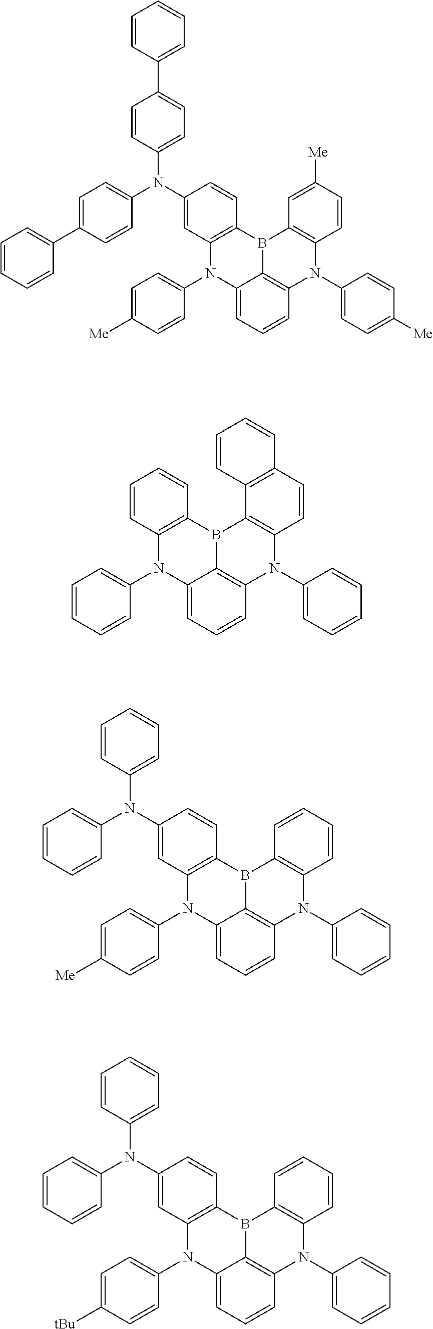 [SMILES: Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1]

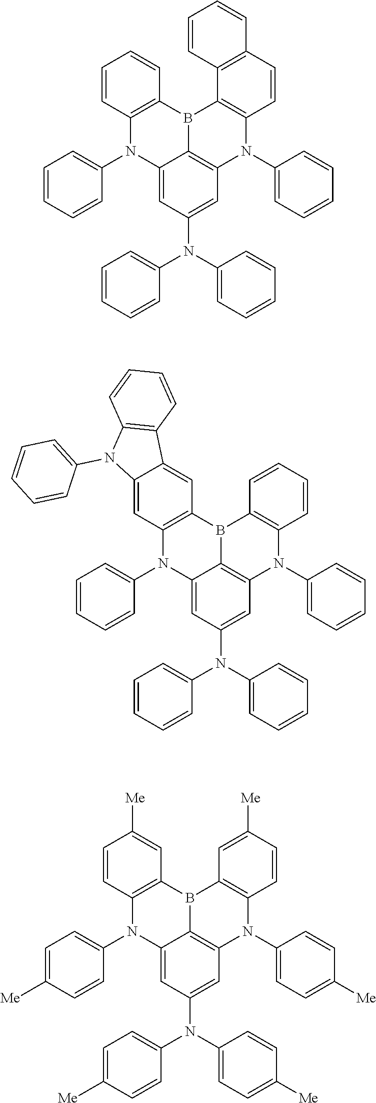 [SMILES: c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1]

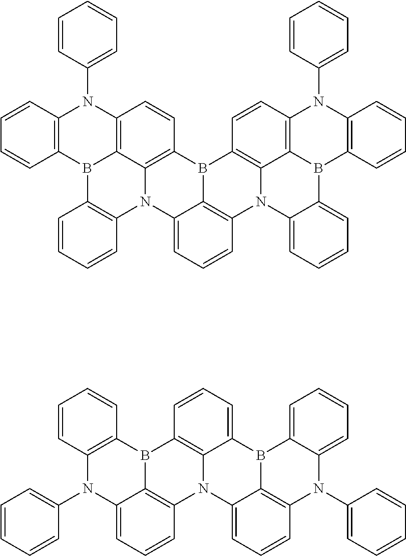 [SMILES: c1ccc(N2c3ccccc3B3c4ccccc4N4c5cccc6c5B(c5ccc2c3c54)c2ccc3c4c2N6c2ccccc2B4c2ccccc2N3c2ccccc2)cc1 ; c1ccc(N2c3ccccc3B3c4cccc5c4N(c4cccc2c43)c2cccc3c2B5c2ccccc2N3c2ccccc2)cc1]

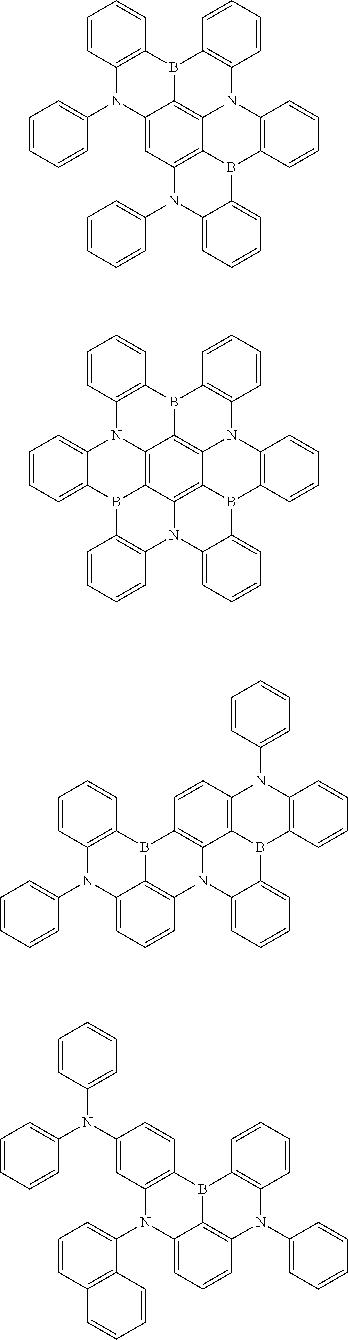 [SMILES: c1ccc(N2c3ccccc3B3c4ccccc4N4c5ccccc5B5c6ccccc6N(c6ccccc6)c6cc2c3c4c65)cc1 ; c1ccc2c(c1)B1c3ccccc3N3c4ccccc4B4c5ccccc5N5c6ccccc6B6c7ccccc7N2c2c1c3c4c5c26 ; c1ccc(N2c3ccccc3B3c4ccc5c6c4N(c4ccccc4B6c4ccccc4N5c4ccccc4)c4cccc2c43)cc1 ; c1ccc(N(c2ccccc2)c2ccc3c(c2)N(c2cccc4ccccc24)c2cccc4c2B3c2ccccc2N4c2ccccc2)cc1]

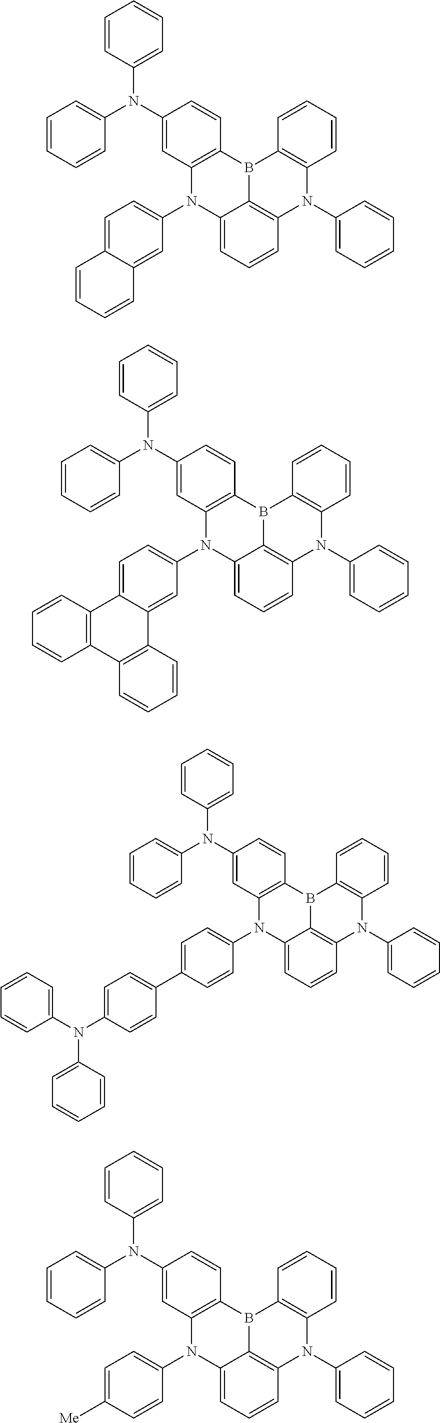

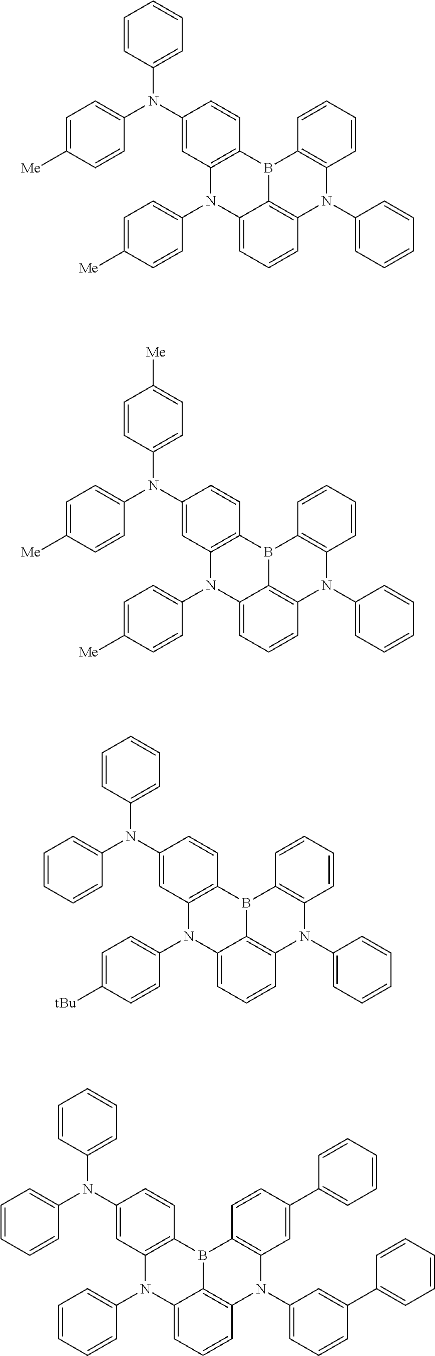 [SMILES: Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

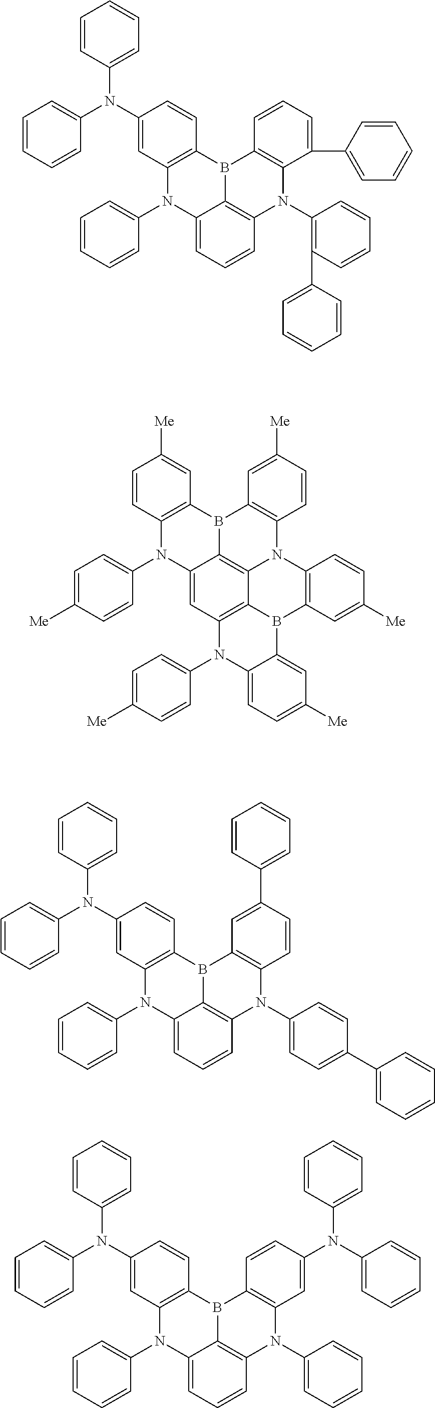 [SMILES: c1ccc(-c2ccccc2N2c3cccc4c3B(c3ccc(N(c5ccccc5)c5ccccc5)cc3N4c3ccccc3)c3cccc(-c4ccccc4)c32)cc1 ; Cc1ccc(N2c3ccc(C)cc3B3c4cc(C)ccc4N4c5ccc(C)cc5B5c6cc(C)ccc6N(c6ccc(C)cc6)c6cc2c3c4c65)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

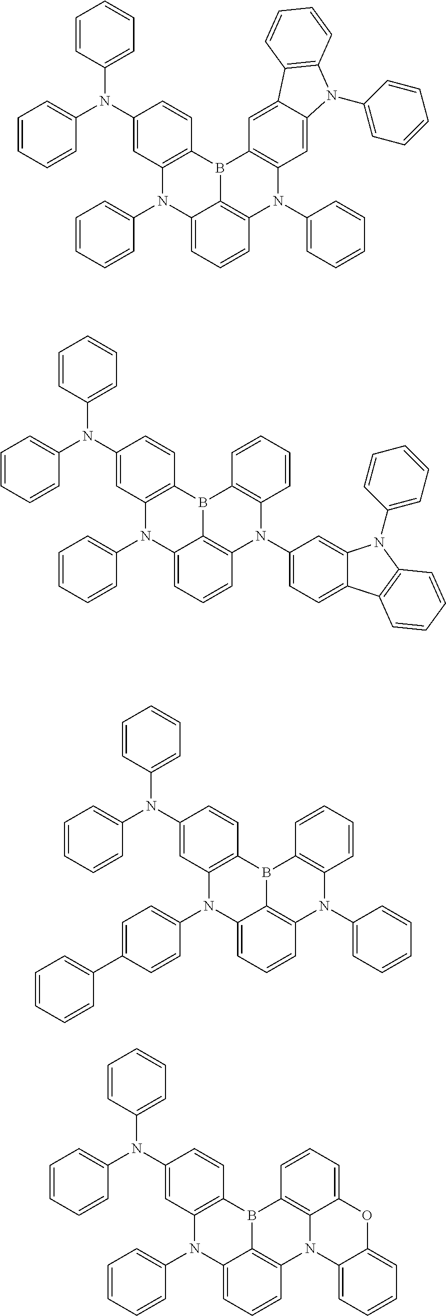 [SMILES: c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1 ; c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2cccc3c2N4c2ccccc2O3)cc1]

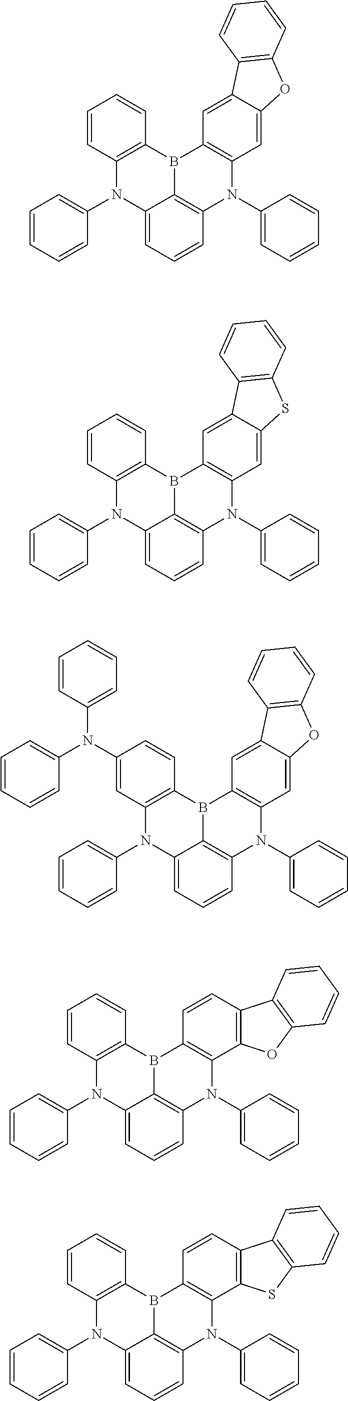 [SMILES: c1ccc(N2c3ccccc3B3c4cc5c(cc4N(c4ccccc4)c4cccc2c43)oc2ccccc25)cc1 ; c1ccc(N2c3ccccc3B3c4cc5c(cc4N(c4ccccc4)c4cccc2c43)sc2ccccc25)cc1 ; c1ccc(N(c2ccccc2)c2ccc3c(c2)N(c2ccccc2)c2cccc4c2B3c2cc3c(cc2N4c2ccccc2)oc2ccccc23)cc1 ; c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1]

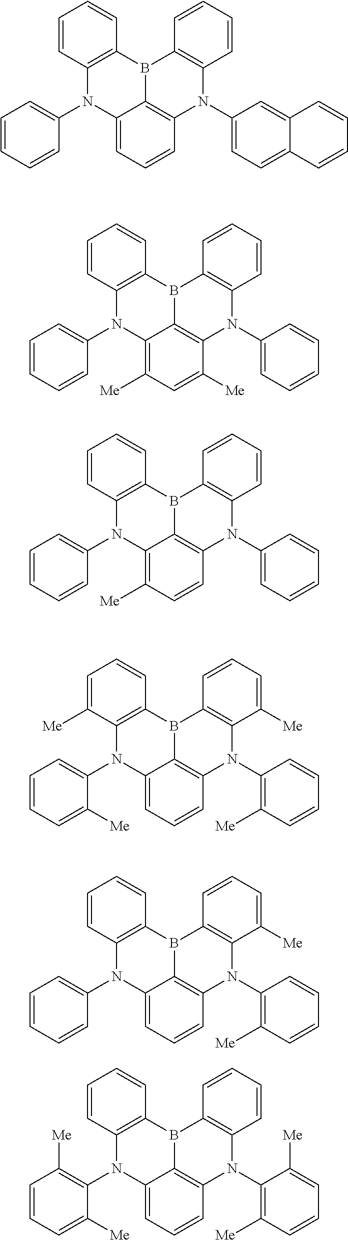

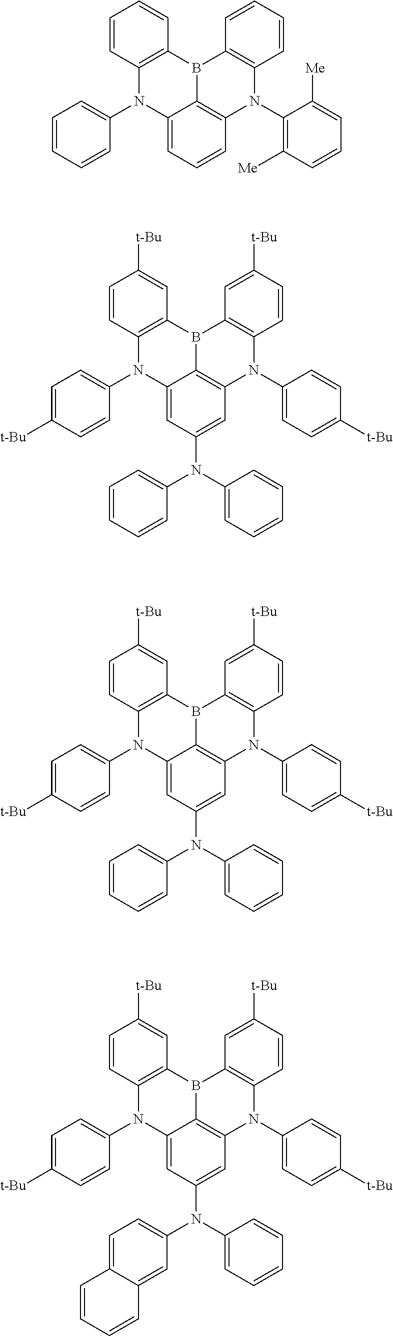 [SMILES: Cc1cccc(C)c1N1c2ccccc2B2c3ccccc3N(c3ccccc3)c3cccc1c32 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1]

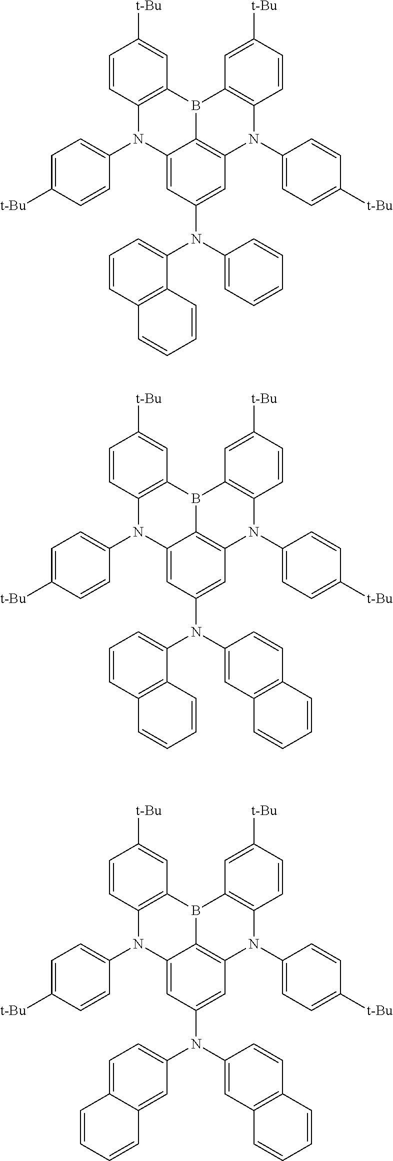 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5ccc6ccccc6c5)cc2c43)cc1]

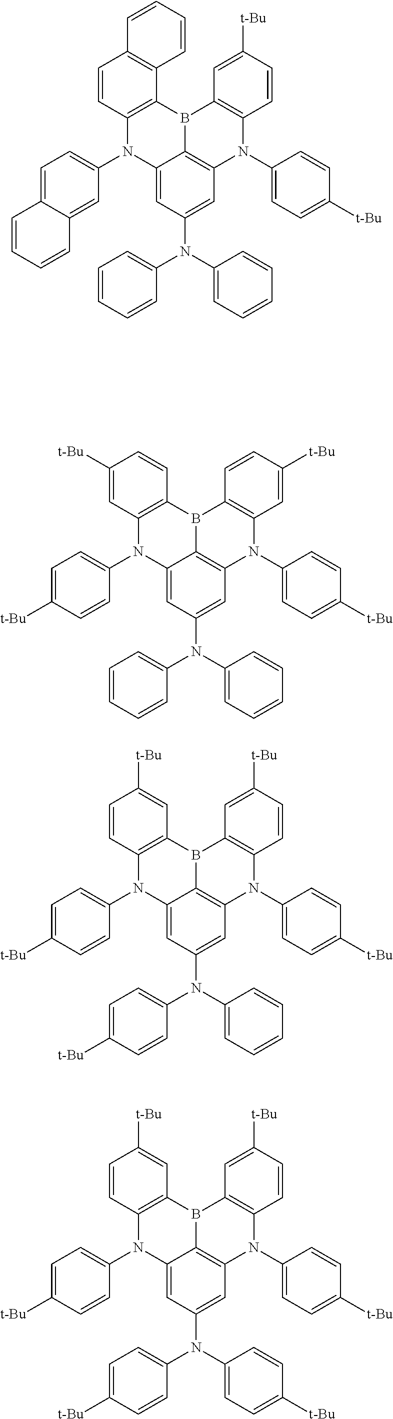

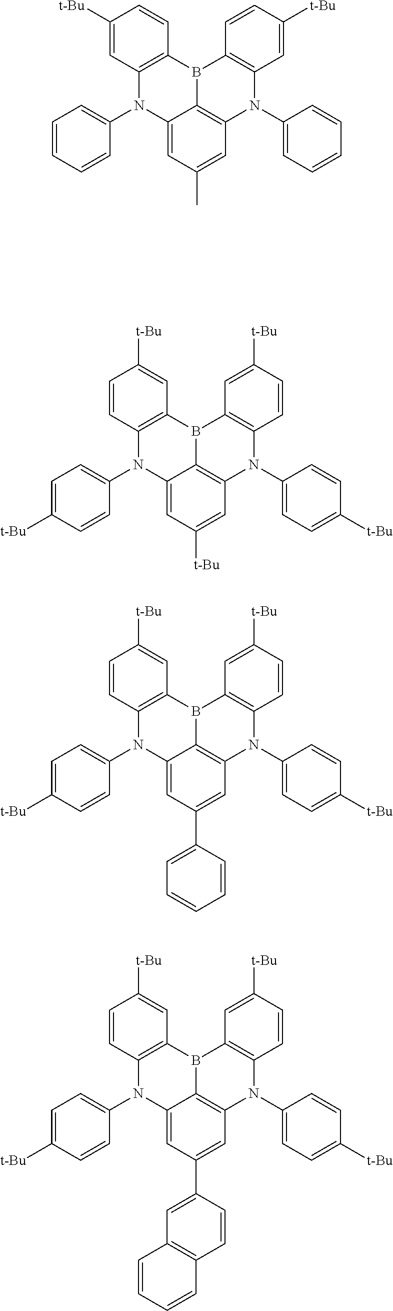 [SMILES: Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1]

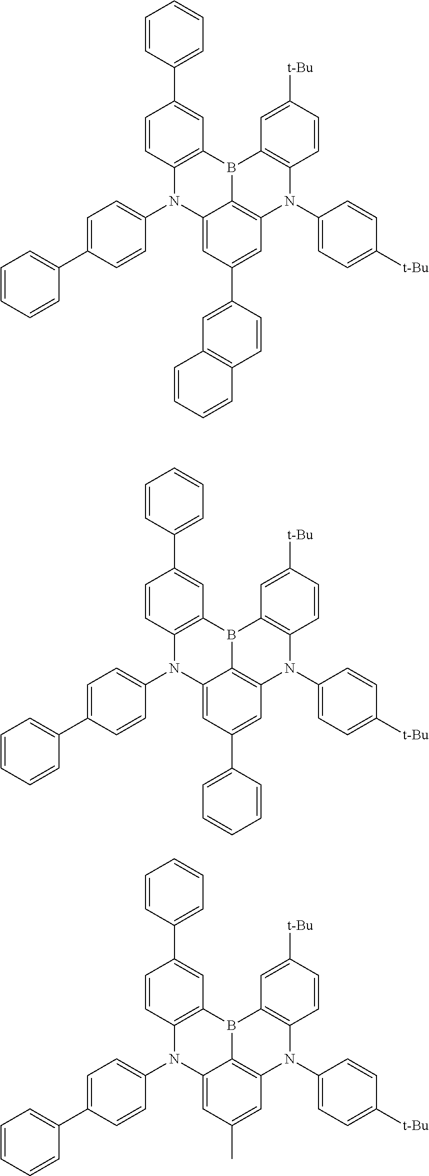 [SMILES: CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1 ; Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1]

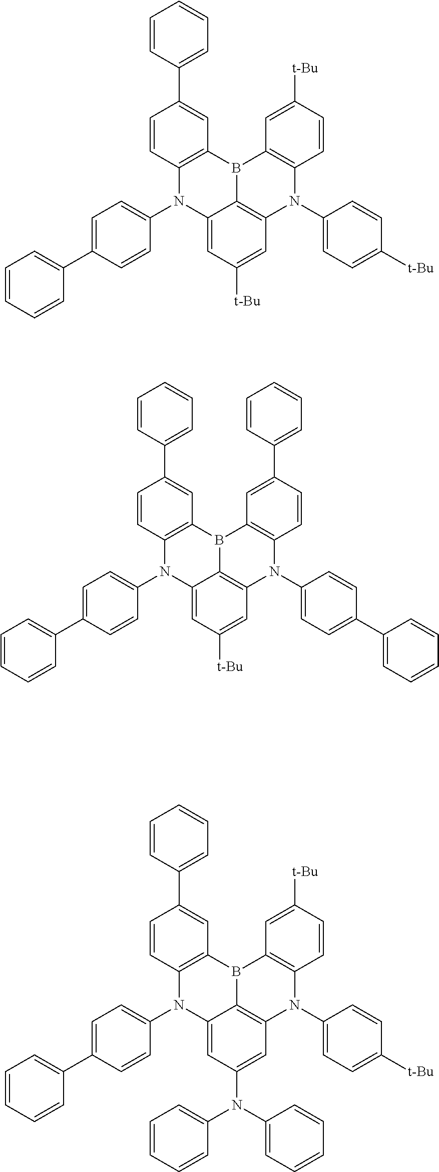 [SMILES: CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1]

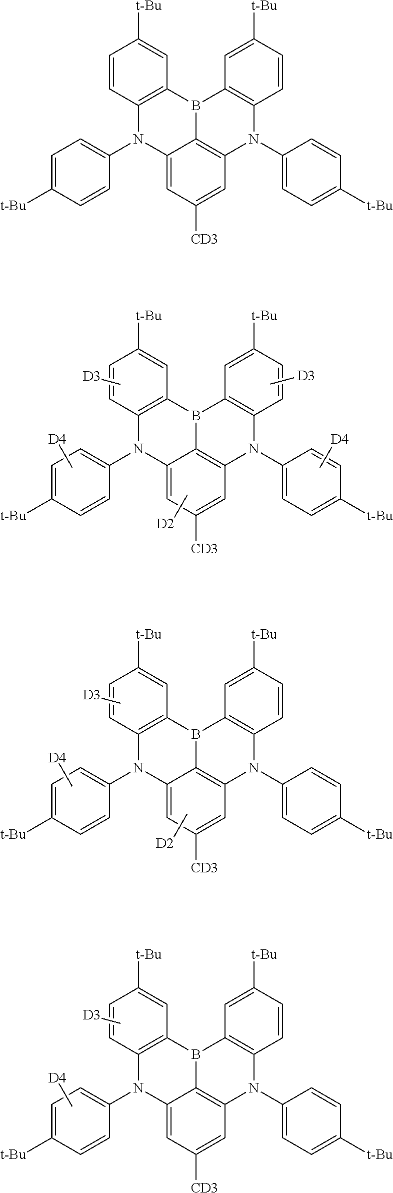

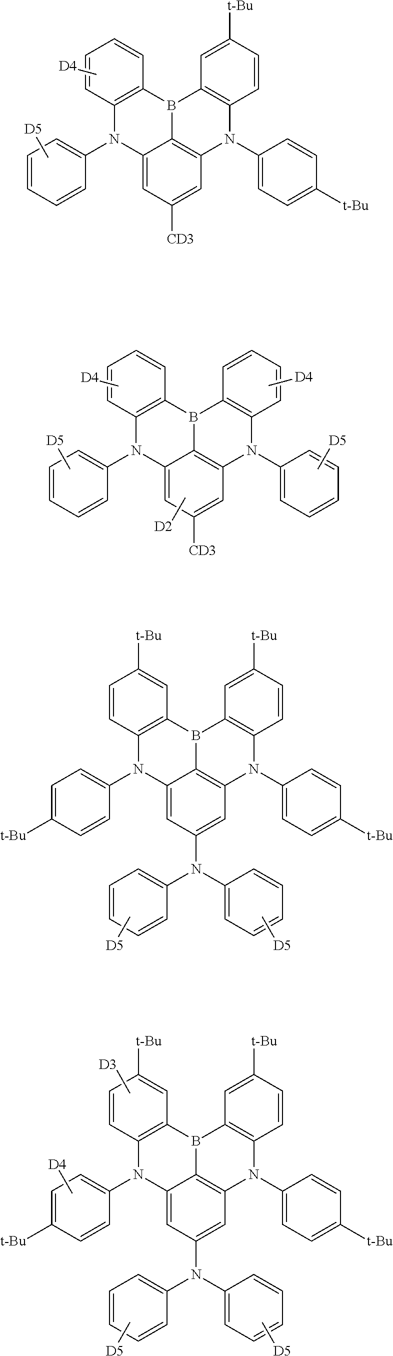

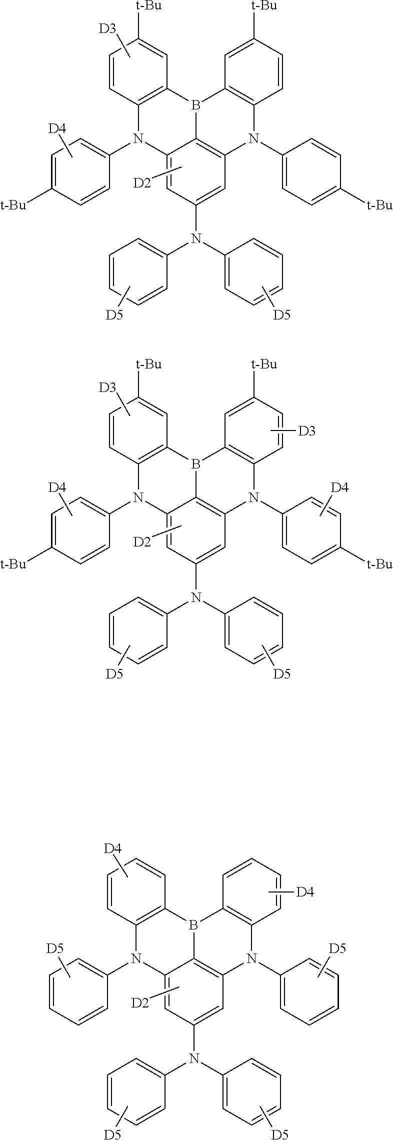

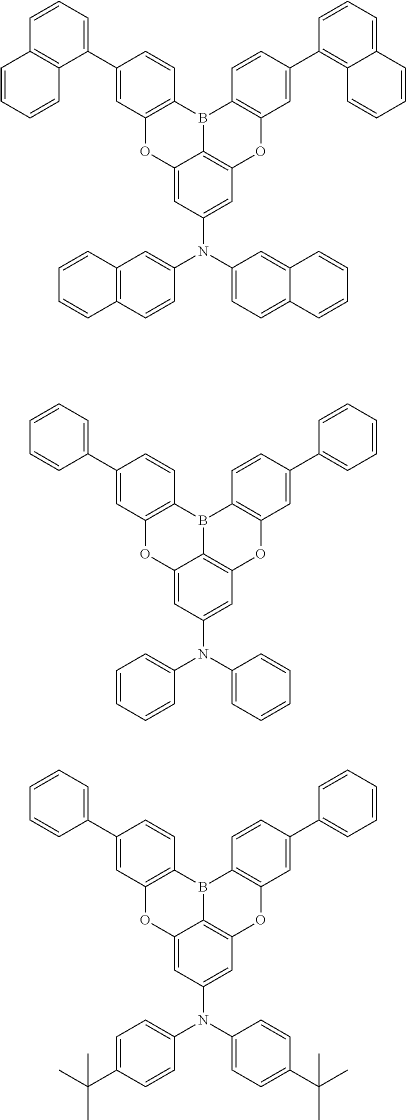 [SMILES: c1ccc2cc(N(c3cc4c5c(c3)Oc3cc(-c6cccc7ccccc67)ccc3B5c3ccc(-c5cccc6ccccc56)cc3O4)c3ccc4ccccc4c3)ccc2c1 ; c1ccc(-c2ccc3c(c2)Oc2cc(N(c4ccccc4)c4ccccc4)cc4c2B3c2ccc(-c3ccccc3)cc2O4)cc1 ; CC(C)(C)c1ccc(N(c2ccc(C(C)(C)C)cc2)c2cc3c4c(c2)Oc2cc(-c5ccccc5)ccc2B4c2ccc(-c4ccccc4)cc2O3)cc1]

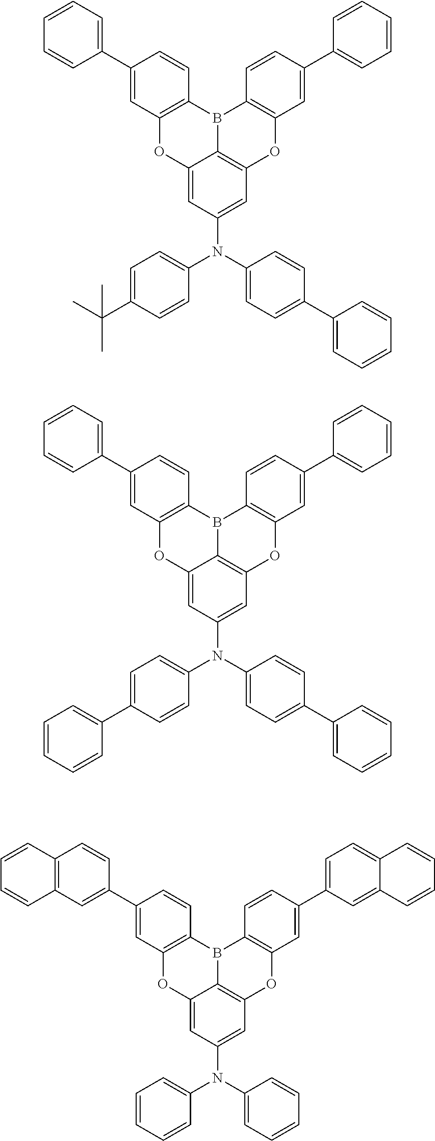

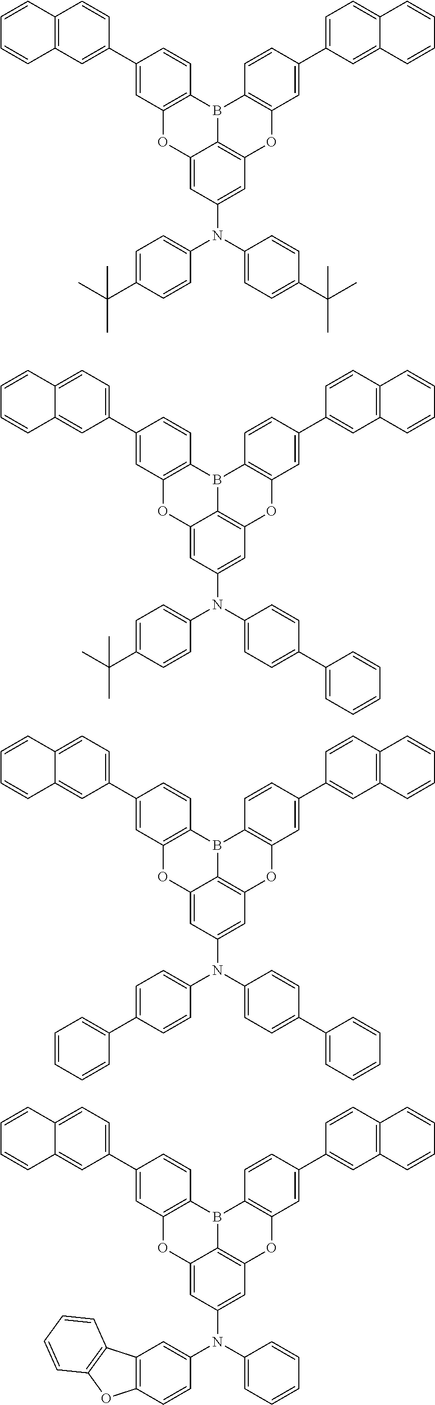

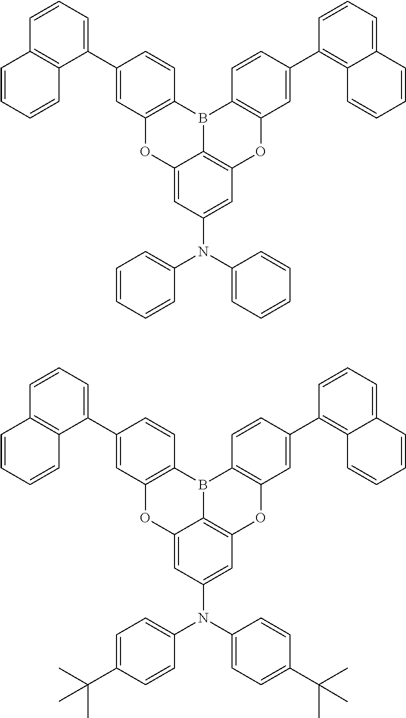 [SMILES: c1ccc(N(c2ccccc2)c2cc3c4c(c2)Oc2cc(-c5cccc6ccccc56)ccc2B4c2ccc(-c4cccc5ccccc45)cc2O3)cc1 ; CC(C)(C)c1ccc(N(c2ccc(C(C)(C)C)cc2)c2cc3c4c(c2)Oc2cc(-c5cccc6ccccc56)ccc2B4c2ccc(-c4cccc5ccccc45)cc2O3)cc1]

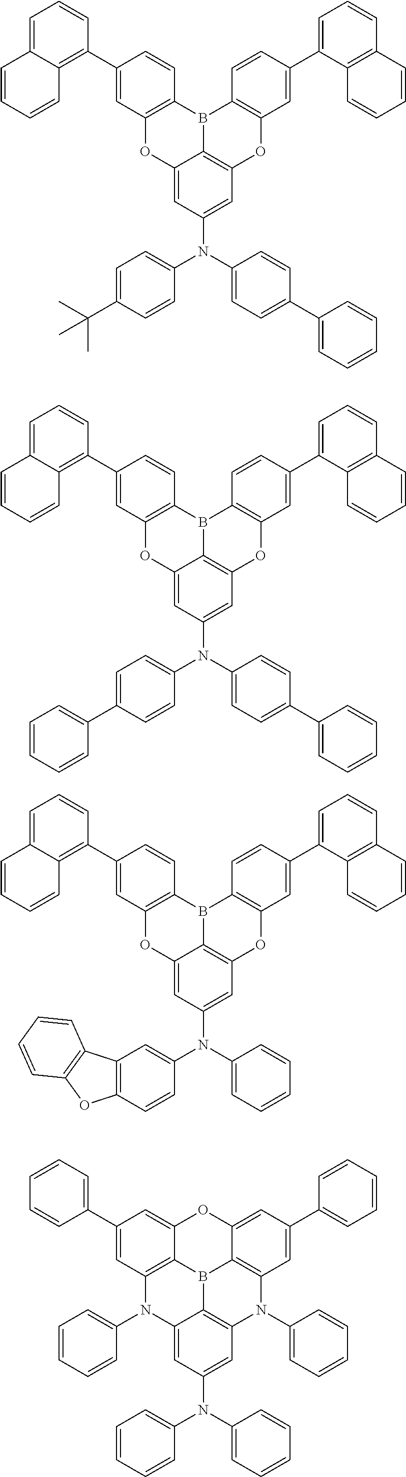

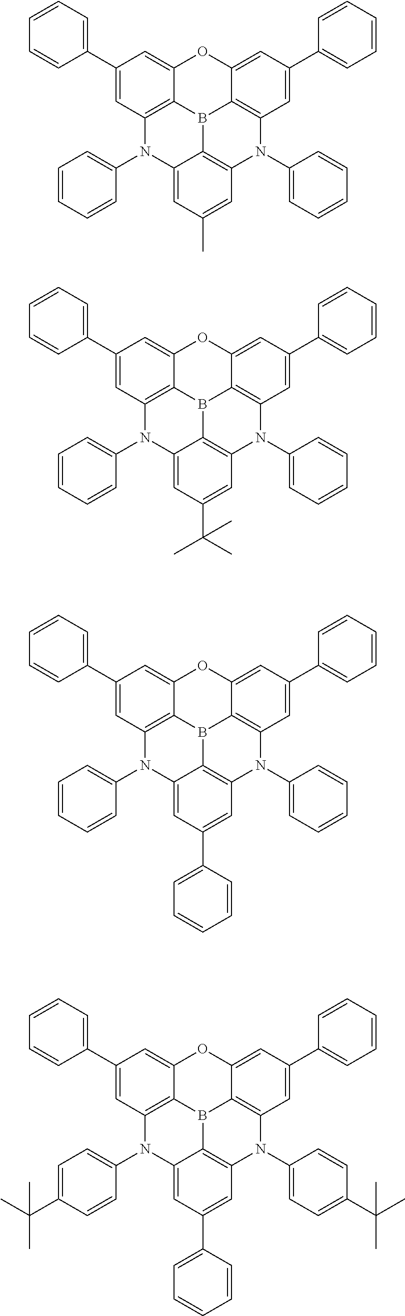 [SMILES: Cc1cc2c3c(c1)N(c1ccccc1)c1cc(-c4ccccc4)cc4c1B3c1c(cc(-c3ccccc3)cc1N2c1ccccc1)O4 ; CC(C)(C)c1cc2c3c(c1)N(c1ccccc1)c1cc(-c4ccccc4)cc4c1B3c1c(cc(-c3ccccc3)cc1N2c1ccccc1)O4 ; c1ccc(-c2cc3c4c(c2)N(c2ccccc2)c2cc(-c5ccccc5)cc5c2B4c2c(cc(-c4ccccc4)cc2N5c2ccccc2)O3)cc1 ; CC(C)(C)c1ccc(N2c3cc(-c4ccccc4)cc4c3B3c5c(cc(-c6ccccc6)cc5N(c5ccc(C(C)(C)C)cc5)c5cc(-c6ccccc6)cc2c53)O4)cc1]

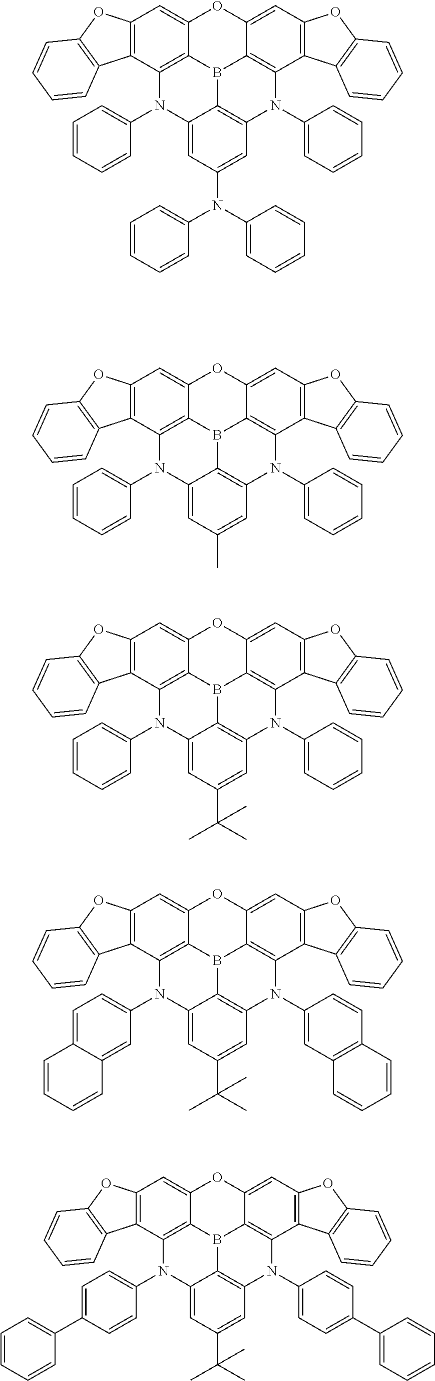 [SMILES: c1ccc(N(c2ccccc2)c2cc3c4c(c2)N(c2ccccc2)c2c5c(cc6oc7ccccc7c26)Oc2cc6oc7ccccc7c6c(c2B45)N3c2ccccc2)cc1 ; Cc1cc2c3c(c1)N(c1ccccc1)c1c4c(cc5oc6ccccc6c15)Oc1cc5oc6ccccc6c5c(c1B34)N2c1ccccc1 ; CC(C)(C)c1cc2c3c(c1)N(c1ccccc1)c1c4c(cc5oc6ccccc6c15)Oc1cc5oc6ccccc6c5c(c1B34)N2c1ccccc1 ; CC(C)(C)c1cc2c3c(c1)N(c1ccc4ccccc4c1)c1c4c(cc5oc6ccccc6c15)Oc1cc5oc6ccccc6c5c(c1B34)N2c1ccc2ccccc2c1 ; CC(C)(C)c1cc2c3c(c1)N(c1ccc(-c4ccccc4)cc1)c1c4c(cc5oc6ccccc6c15)Oc1cc5oc6ccccc6c5c(c1B34)N2c1ccc(-c2ccccc2)cc1]

In the compounds above, D2 to D5 represent that 2 to 5 hydrogens are replaced with deuterium, respectively. For example, D5 means substituted with five (5) deuterium.

At least one of the compounds H1-1 to H1-135 and at least one of the specific compounds of formula 2 above may be combined to be used in the organic electroluminescent device.

The compound represented by formula 1 according to the present disclosure may be prepared by a synthetic method known to one skilled in the art, for example, by referring to Korean Patent Appl. Laid-Open No. 2015-0010016 (published on Jan. 28, 2015), etc.

The compound represented by formula 2 according to the present disclosure may be prepared by a synthetic method known to one skilled in the art, for example, by referring to Korean Patent No. 1876763 (registered on Jul. 4, 2018), Japanese Patent No. 5935199 (registered on May 20, 2016), Korean Patent Appl. Laid-Open No. 2017-0130434 (published on Nov. 28, 2017), etc.

Hereinafter, an organic electroluminescent device comprising the aforementioned plurality of light-emitting materials will be described.

The organic electroluminescent device according to one embodiment of the present disclosure comprises a first electrode, a second electrode, and at least one organic layer interposed between the first and the second electrodes. The organic layer may comprise a light-emitting layer, and the light-emitting layer may include a light-emitting material(s) comprising at least one first compound represented by formula 1, and at least one second compound represented by formula 2.

According to one embodiment, the host compound represented by formula 1 and the dopant compound represented by formula 2 may be included in the same organic layer, or may be included in different organic layers, respectively.

The light-emitting layer is a layer comprising a host(s) and a dopant(s) from which light is emitted, and can be a single layer or a multi-layer in which two or more layers are stacked. Here, the host mainly has a function of promoting recombination of electrons and holes and confining excitons in the light-emitting layer, and the dopant has a function of efficiently emitting light of excitons obtained by recombination. The dopant compound of the light-emitting layer may be doped to less than 25% by weight, preferably less than 17% by weight based on the total amount of the host and dopant compounds.

One of the first and the second electrodes may be an anode, and the other may be a cathode. The organic layer comprises a light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. The second electrode may be a transflective electrode or a reflective electrode, and may be a top emission type, a bottom emission type, or a both-sides emission type, depending on the materials. In addition, the hole injection layer may be additionally doped with a p-dopant, and the electron injection layer may be additionally doped with an n-dopant.

The organic electroluminescent device according to one embodiment of the present disclosure may comprise an anode, a cathode, and at least one organic layer between the anode and the cathode, in which the organic layer may comprise a light-emitting layer and a hole transport zone between the anode and the light-emitting layer. The light-emitting layer may comprise a plurality of light-emitting materials according to the present disclosure, and the hole transport zone comprises a compound represented by the following formula 3.

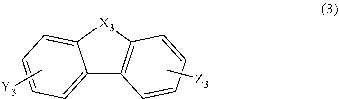

In formula 3,
X₃ represents NR₄₁, O, S, or CR₄₂R₄₃;
Y₃, Z₃ and R₄₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; and R₄₂ and R₄₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or R₄₂ and R₄₃ may be linked to each other to form a ring(s).

The organic layer may further comprise an amine-based compound and/or an azine-based compound in addition to the light-emitting material(s) of the present disclosure. Specifically, the hole injection layer, the hole transport layer, the hole auxiliary layer, the light-emitting layer, the light-emitting auxiliary layer, or the electron blocking layer may comprise an amine-based compound, e.g., an arylamine-based compound, a styrylarylamine-based compound, etc., as a hole injection material, a hole transport material, a hole auxiliary material, a light-emitting material, a light-emitting auxiliary material, and an electron blocking material. In addition, the electron transport layer, the electron injection layer, the electron buffer layer, and the hole blocking layer may comprise an azine-based compound as an electron transport material, an electron injection material, an electron buffer material, and a hole blocking material.

In addition, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal.

A hole injection layer, a hole transport layer, an electron blocking layer, or a combination thereof may be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. The electron blocking layer may be located between the hole transport layer (or the hole injection layer) and the light-emitting layer, and may block the overflow of electrons from the light-emitting layer to trap the excitons in the light-emitting layer to prevent light leakage. The hole transport layer or the electron blocking layer may be multilayers, wherein each of the multilayers may use a plurality of compounds.

An electron buffer layer, a hole blocking layer, an electron transport layer, an electron injection layer, or a combination thereof may be used between the light-emitting layer and the cathode. The electron buffer layer may be multilayers in order to control the injection of the electrons and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

A light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes.

In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifespan of the organic electroluminescent device.

In the organic electroluminescent device of the present disclosure, at least one layer selected from a chalcogenide layer, a metal halide layer and a metal oxide layer (hereinafter, “a surface layer”) may be preferably placed on an inner surface(s) of at least one of a pair of electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer may provide operation stability for the organic electroluminescent device. Preferably, the chalcogenide includes SiO_{X}(1≤X≤2), AlO_{X}(1≤X≤1.5), SiON, SiAION, etc.; the metal halide includes LiF, MgF₂, CaF₂, a rare earth metal fluoride, etc.; and the metal oxide includes Cs₂O, Li₂O, MgO, SrO, BaO, CaO, etc.

In the organic electroluminescent device of the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be used as a charge-generating layer to prepare an organic electroluminescent device having two or more light-emitting layers and emitting white light.

Various structures have been proposed for the white organic electroluminescent device, for example, a side-by-side structure or a stacking structure depending on the arrangement of R (red), G (green) or YG (yellow green), and B (blue) light emitting parts, or a color conversion material (CCM) method, etc. The plurality of light-emitting materials of the present disclosure may also be applied to such white organic electroluminescent device.

The plurality of light-emitting materials according to one embodiment of the present disclosure may also be applied to the organic electroluminescent device comprising a QD.

The present disclosure may provide a display device by using a plurality of light-emitting materials according to one embodiment of the present disclosure. That is, it is possible to produce a display device or a lighting device by using the compounds of the present disclosure. Specifically, it is possible to produce a display device, e.g., a display device for smartphones, tablets, notebooks, PCs, TVs, or cars, or a lighting device, e.g., an outdoor or indoor lighting device, by using the compounds of the present disclosure.

Each layer of the organic electroluminescent device of the present disclosure may be formed by one of the dry film-forming methods such as vacuum evaporation, sputtering, plasma and ion plating methods, or wet film-forming methods such as ink jet printing, nozzle printing, slot coating, spin coating, dip coating, and flow coating methods.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

The dopant and host compounds of the present disclosure may be co-evaporated or mixture-evaporated. The co-evaporation is a mixed deposition method in which two or more isomer materials are placed in a respective individual crucible source and a current is applied to both cells at the same time to evaporate the materials. The mixture-evaporation is a mixed deposition method in which two or more isomer materials are mixed in one crucible source before evaporating them, and a current is applied to the cell to evaporate the materials.

Hereinafter, the preparation method of the organic electroluminescent compound according to the present disclosure and the organic electroluminescent device comprising a plurality of the light-emitting materials according to the present disclosure, and the properties thereof will be explained in detail.

### Example 1: Preparation of Compound H1-1

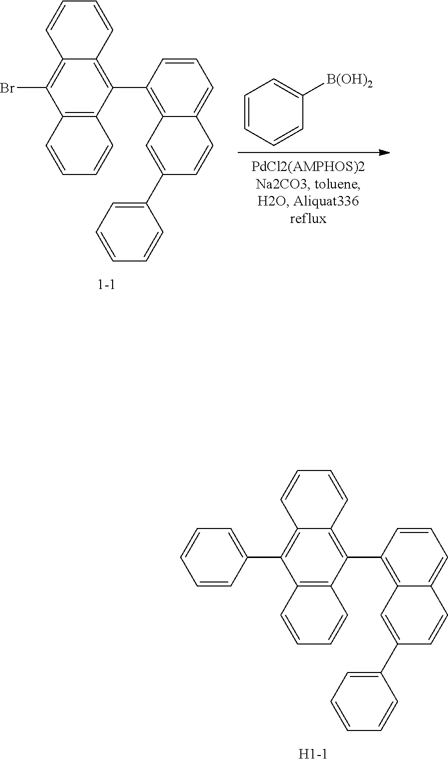

In a flask, compound 1-1 (10 g, 21.7 mmol), phenylboronic acid (3.2 g, 26.1 mmol), PdCl₂(Amphos)₂ (0.77 g, 1.1 mmol), Na₂CO₃ (4.62 g, 43.6 mmol), toluene 150 mL, distilled water 50 mL, and Aliquat336 (0.44 g, 1.1 mmol) were added and stirred under reflux for 30 minutes. After cooling to room temperature, distilled water was added thereto. An organic layer was extracted with dichloromethane and dried by magnesium sulfate. The organic layer was distilled under reduced pressure and separated by column chromatography to obtain compound H1-1 (9 g, 90.9%).

| | MW | M.P. |
|---|---|---|
| H1-1 | 456.59 | 242° C. |

### Example 2: Preparation of Compound H1-6

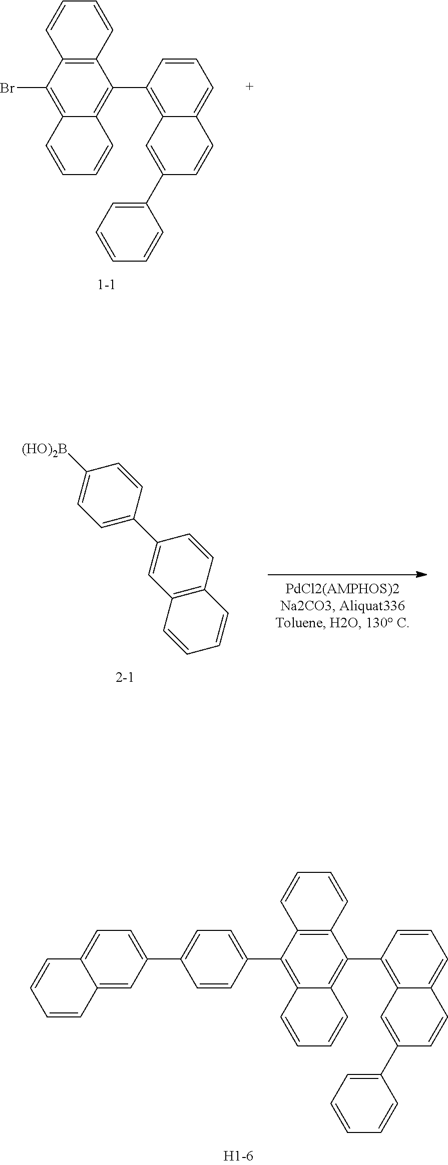

In a flask, compound 1-1 (10 g, 21.7 mmol), compound 2-1 (6.5 g, 26.1 mmol), PdCl₂(Amphos)₂ (0.49 g, 0.7 mmol), Na₂CO₃ (6.9 g, 65.4 mmol), toluene 200 mL, distilled water 65 mL, and Aliquat336 (0.5 mL, 1.1 mmol) were added and stirred at 130° C. for 3 hours. After cooling to room temperature, distilled water was added thereto. An organic layer was extracted with dichloromethane and dried by magnesium sulfate. The organic layer was distilled under reduced pressure and separated by column chromatography to obtain compound H1-6 (9.2 g, 72.4%).

### Example 3: Preparation of Compound H1-7

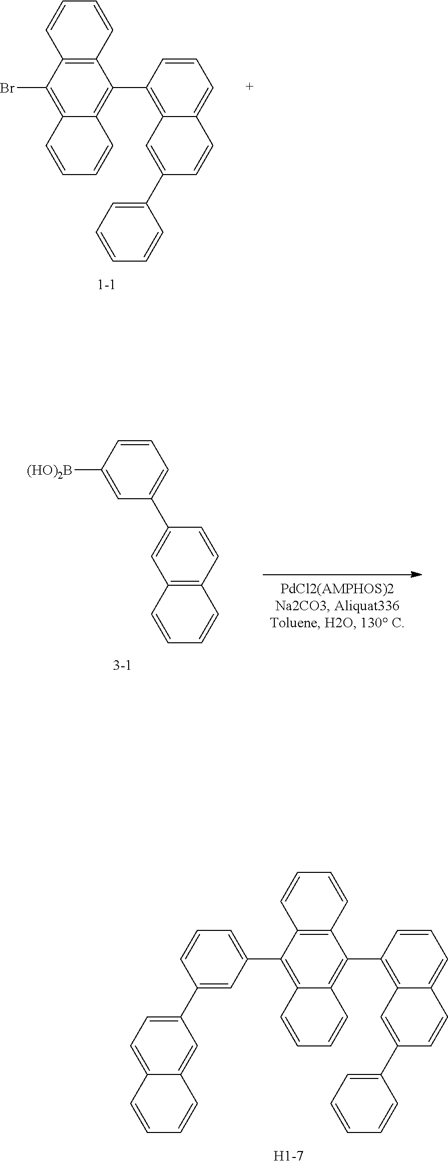

In a flask, compound 1-1 (10 g, 21.7 mmol), compound 3-1 (6.5 g, 26.1 mmol), PdCl₂(Amphos)₂ (0.49 g, 0.7 mmol), Na₂CO₃ (6.9 g, 65.4 mmol), toluene 200 mL, distilled water 65 mL, and Aliquat336 (0.5 mL, 1.1 mmol) were added and stirred at 130° C. for 3 hours. After cooling to room temperature, distilled water was added thereto. An organic layer was extracted with dichloromethane and dried by magnesium sulfate. The organic layer was distilled under reduced pressure and separated by column chromatography to obtain compound H1-7 (9.9 g, 77.9%).

### Example 4: Preparation of Compound H1-48

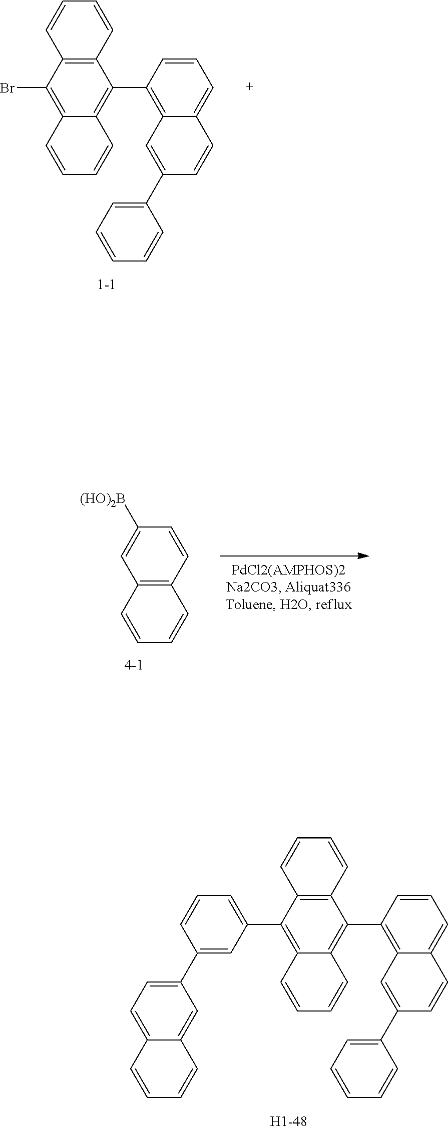

In a flask, compound 1-1 (10 g, 21.7 mmol), compound 4-1 (4.5 g, 26.1 mmol), PdCl₂(Amphos)₂ (0.77 g, 1.1 mmol), Na₂CO₃ (4.62 g, 43.6 mmol), toluene 150 mL, distilled water 50 mL, and Aliquat336 (0.44 g, 1.1 mmol) were added and stirred under reflux for 1 hour. After cooling to room temperature, distilled water was added thereto. An organic layer was extracted with dichloromethane and dried by magnesium sulfate. The organic layer was distilled under reduced pressure and separated by column chromatography to obtain compound H1-48 (10 g, 90.9%).

### Device Examples 1 to 5: Producing an OLED Comprising a Plurality of Light-Emitting Material According to the Present Disclosure

An OLED was produced using the plurality of the light-emitting material according to the present disclosure, as follows: A transparent electrode indium tin oxide (ITO) thin film (10 Ω/sq) on a glass substrate for an OLED (GEOMATEC CO., LTD., Japan) was subjected to an ultrasonic washing with acetone, ethanol and distilled water, sequentially, and then was stored in isopropanol. The ITO substrate was mounted on a substrate holder of a vacuum vapor deposition apparatus. Compound HT-1 was introduced into one cell of the vacuum vapor deposition apparatus, and compound HI-1 was introduced into another cell. The pressure in the chamber of the apparatus was then controlled to 10⁻⁶ torr. Thereafter, the two materials were evaporated so that compound HI-1 was deposited in a doping amount of 3% by weight based on the total amount of compound HT-1 and compound HI-1, thereby depositing a hole injection layer having a thickness of 10 nm on the ITO substrate. Next, compound HT-1 was introduced into a cell of the vacuum vapor deposition apparatus and was evaporated by applying an electric current to the cell, thereby depositing a first hole transport layer having a thickness of 75 nm on the hole injection layer. Compound HT-2 was then introduced into another cell of the vacuum vapor deposition apparatus and was evaporated by applying an electric current to the cell, thereby depositing a second hole transport layer having a thickness of 5 nm on the first hole transport layer. After forming the hole injection layer and the hole transport layers, a light-emitting layer was deposited thereon as follows: Compound H1-1 was introduced into a cell of the vacuum vapor deposition apparatus as a host of a light-emitting layer and compound BD-2 was introduced into another cell as a dopant. The two materials were evaporated so that the dopant was deposited in a doping amount of 2% by weight based on the total amount of the host and dopant to deposit a light-emitting layer having a thickness of 20 nm on the second hole transport layer. Subsequently, compound ET-1 was deposited as a hole blocking layer to a thickness of 5 nm. Next, in two other cells compound ET-2 and compound EI-1 were evaporated at a rate of 1:1 to deposit an electron transport layer having a thickness of 30 nm on the light-emitting layer. After depositing compound EI-1 as an electron injection layer having a thickness of 2 nm, an Al cathode having a thickness of 80 nm was deposited by another vacuum vapor deposition apparatus to produce an OLED.

### Comparative Example 1: Producing an OLED Comprising a Conventional Plurality of Light-Emitting Materials

An OLED was produced in the same manner as in Device Examples 1 to 5, except that compound BD-1 was used instead of compound BD-2 as the dopant material of the light-emitting layer.

Table 1 shows the measurements of CIE color coordinate, electro-luminance wavelength, and full width at half maximum (FWHM) based on 1,000 nit, and the minimum time taken to be reduced from 100% to 90% of the luminance (lifespan; T90) based on a luminance of 2,000 nit of the organic electroluminescent device of the Device Examples 1 to 5 and Comparative Example 1.

TABLE 1

| | | | Electro-luminance | Full width at | CIE color | lifespan |
|---|---|---|---|---|---|---|
| | | | wavelength (nm) | half maximum | coordinate | (T90) |
| | Host | Dopant | @ 1000 nit | | | @ 2000 nit |
| Device | H1-1 | BD-2 | 456 | 27 | (0.138, 0.065) | 136 |
| Example 1 | | | | | | |
| Device | H1-6 | BD-2 | 457 | 28 | (0.137, 0.065) | 212 |
| Example 2 | | | | | | |
| Device | H1-7 | BD-2 | 457 | 27 | (0.138, 0.063) | 165 |
| Example 3 | | | | | | |
| Device | H1-48 | BD-2 | 457 | 26 | (0.138, 0.061) | 194 |
| Example 4 | | | | | | |
| Device | H1-63 | BD-2 | 456 | 27 | (0.139, 0.061) | 73 |
| Example 5 | | | | | | |
| Comparative | H1-1 | BD-1 | 460 | 44 | (0.132, 0.118) | 61 |
| Example 1 | | | | | | |

HT-1 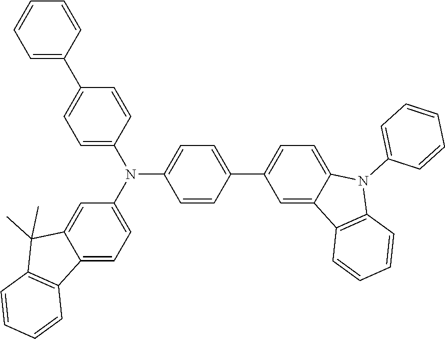 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]
HI-1 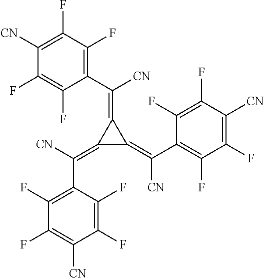
HT-2 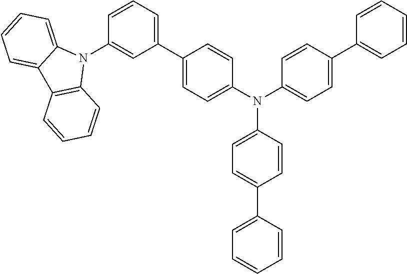 [SMILES: c1ccc(-c2ccc(N(c3ccc(-c4ccccc4)cc3)c3ccc(-c4cccc(-n5c6ccccc6c6ccccc65)c4)cc3)cc2)cc1]
ET-1 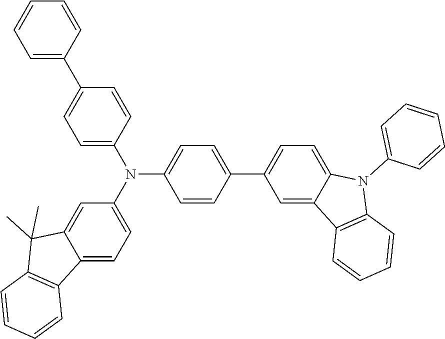 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]
ET-2 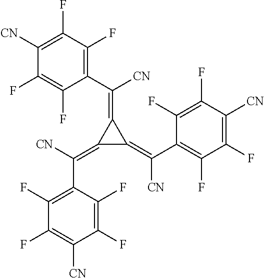
EI-1 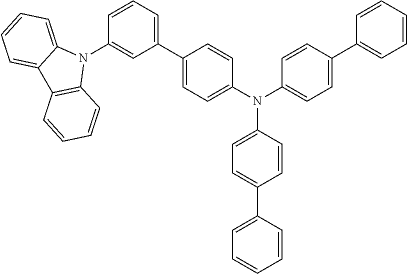 [SMILES: c1ccc(-c2ccc(N(c3ccc(-c4ccccc4)cc3)c3ccc(-c4cccc(-n5c6ccccc6c6ccccc65)c4)cc3)cc2)cc1]
BD-1 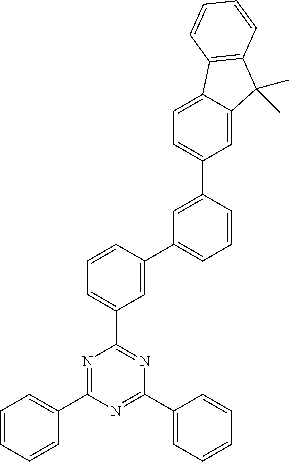 [SMILES: CC1(C)c2ccccc2-c2ccc(-c3cccc(-c4cccc(-c5nc(-c6ccccc6)nc(-c6ccccc6)n5)c4)c3)cc21]
BD-2

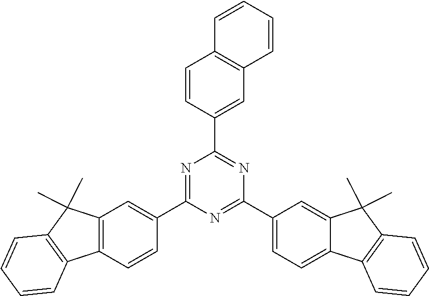 [SMILES: CC1(C)c2ccccc2-c2ccc(-c3nc(-c4ccc5c(c4)C(C)(C)c4ccccc4-5)nc(-c4ccc5ccccc5c4)n3)cc21]

From Table 1 above, it can be seen that the CIE color coordinate of the OLED produced by using the specific combination of a plurality of light-emitting materials according to the present disclosure emitted blue light with deeper color compared to the conventional OLED. Due to such a deep blue light emission, it is possible to express a wider range of colors than the conventional blue light emission in the display implementation. In addition, a blue light-emitting organic electroluminescent device with a longer lifespan than the prior art can be produced according to the present disclosure, which enables to maintain a balance of the lifespan of a red or green light-emitting organic electroluminescent device.

1. A plurality of light-emitting materials comprising at least one of first compounds and at least one of second compounds, wherein the first compound is represented by the following formula 1, and the second compound is represented by the following formula 2: 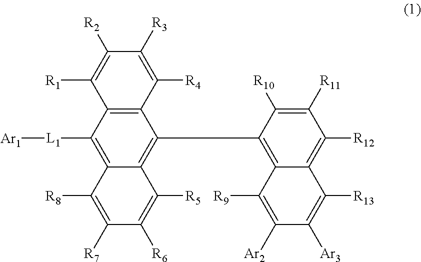
wherein,
L₁ represents a single bond, a substituted or unsubstituted (C6-C30)arylene, or a substituted or unsubstituted (5- to 30-membered)heteroarylene;
Ar₁ represents a substituted or unsubstituted (C6-C30)aryl, or a substituted or unsubstituted (5- to 30-membered)heteroaryl;
Ar₂ and Ar₃, each independently, represent hydrogen, deuterium, a substituted or unsubstituted (C6-C30)aryl, or a substituted or unsubstituted (5- to 30-membered)heteroaryl, with a proviso that Ar₂ and Ar₃ are not simultaneously hydrogen; and
R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino,
or a substituted or unsubstituted (C1-C30)alkyl(C6-C30)arylamino; 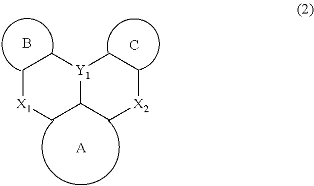
wherein,
ring A, ring B, and ring C, each independently, represent a substituted or unsubstituted (C6-C30)aryl, or a substituted or unsubstituted (3- to 50-membered)heteroaryl, wherein ring B and ring C may be linked to each other to form a ring;
Y₁ represents B;
X₁ and X₂, each independently, represent NR or O; and
R each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, aryl, or a substituted or unsubstituted (C1-C30)alkyl(C6-C30)arylamino; or may be linked to at least one of ring A, ring B, and ring C to form a ring(s).

2. The plurality of light-emitting materials according to claim 1, wherein the substituents of the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, or the substituted alkylarylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with at least one of a (C1-C30)alkyl(s), a (C6-C30)aryl(s) and a di(C6-C30)arylamino(s); a (C6-C30)aryl unsubstituted or substituted with at least one of a (C1-C30)alkyl(s), a (5- to 30-membered)heteroaryl(s) and a di(C6-C30)arylamino(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with at least one of a (C1-C30)alkyl(s), a (5- to 30-membered)heteroaryl(s) and a di(C6-C30)arylamino(s); a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of light-emitting materials according to claim 1, wherein L₁ in formula 1 represents a single bond, or any one selected from the following Group 1: 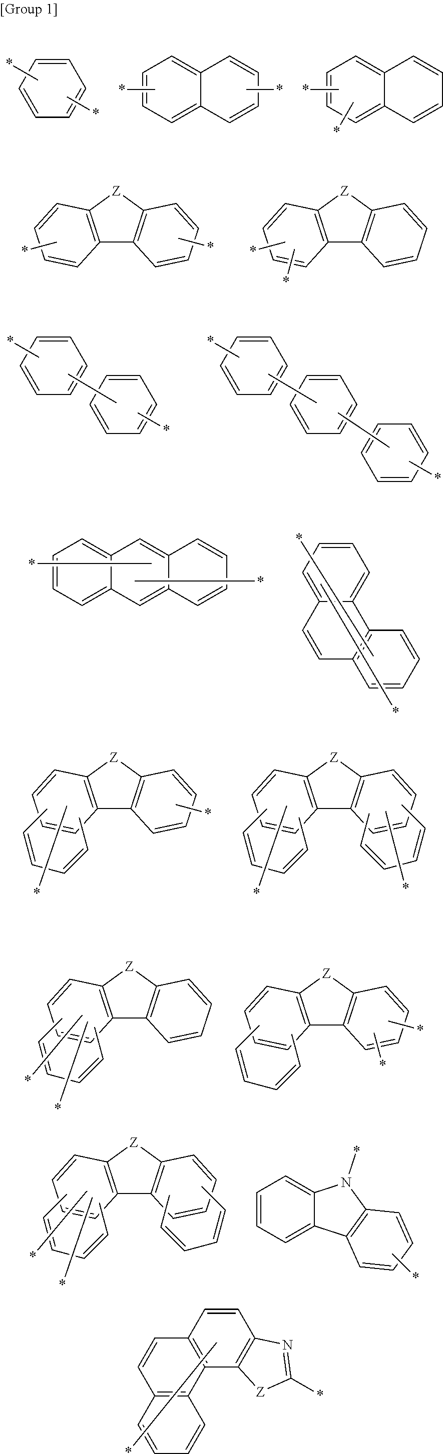
wherein,
Z represents O, S, NR₁₀₁, CR₁₀₂R₁₀₃, or SiR₁₀₄R₁₀₅;
R₁₀₁ to R₁₀₅, each independently, represent a substituted or unsubstituted (C1-C30)alkyl, a substituted or unsubstituted (C6-C30)aryl, or a substituted or unsubstituted (3- to 30-membered)heteroaryl; or may be linked to an adjacent substituent to form a ring(s); and
* represents a bonding site with anthracene backbone and Ar₁.

4. The plurality of light-emitting materials according to claim 1, wherein Ar₁ in formula 1 is selected from the substituents listed in the following Group 2, and Ar₂ and Ar₃, each independently, represent hydrogen, deuterium, or selected from the substituents listed in the following Group 2, with a proviso that Ar₂ and Ar₃ are not simultaneously hydrogen: 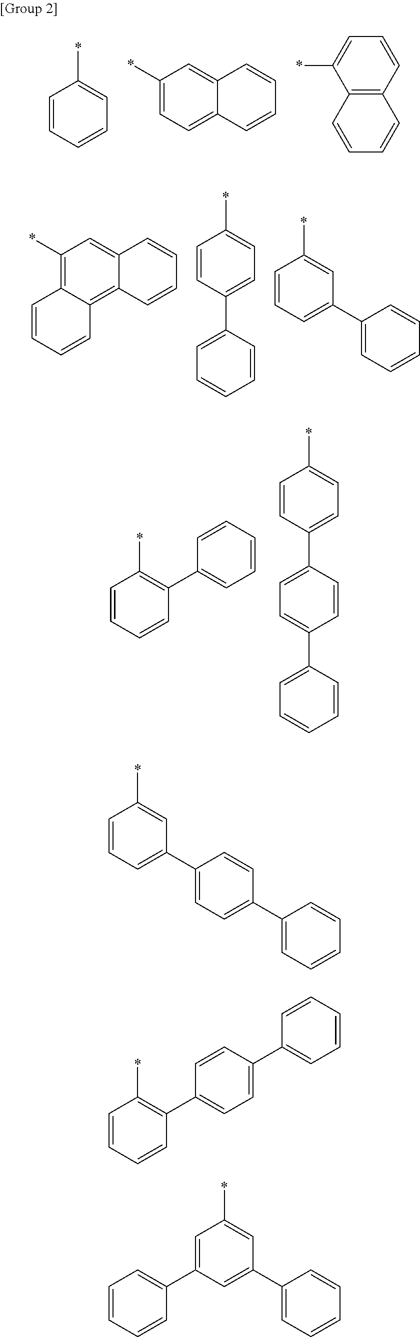 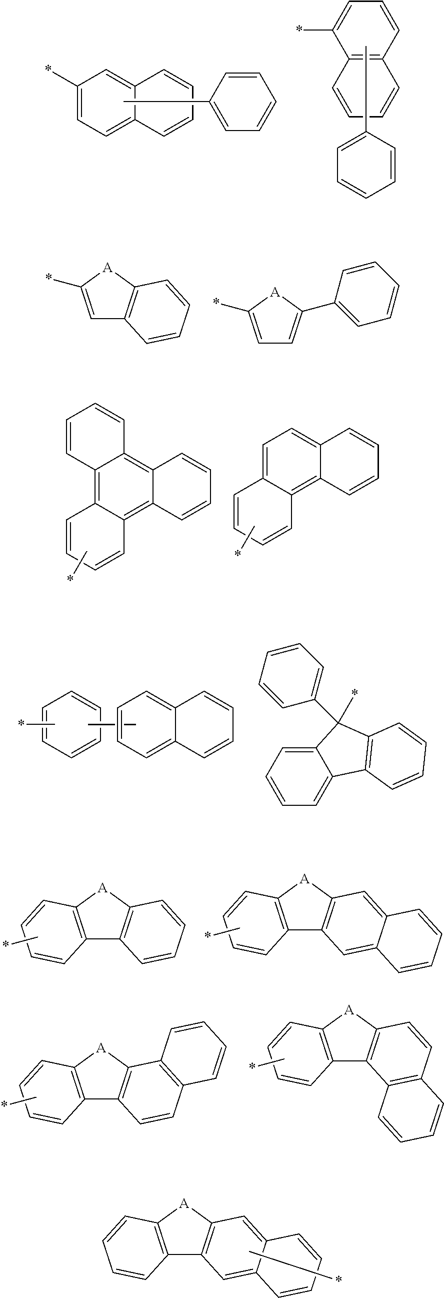 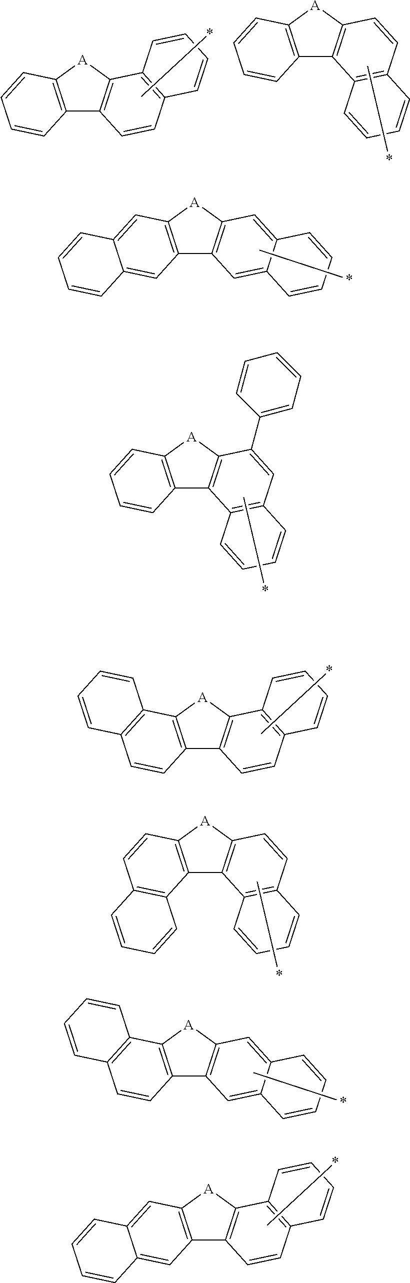 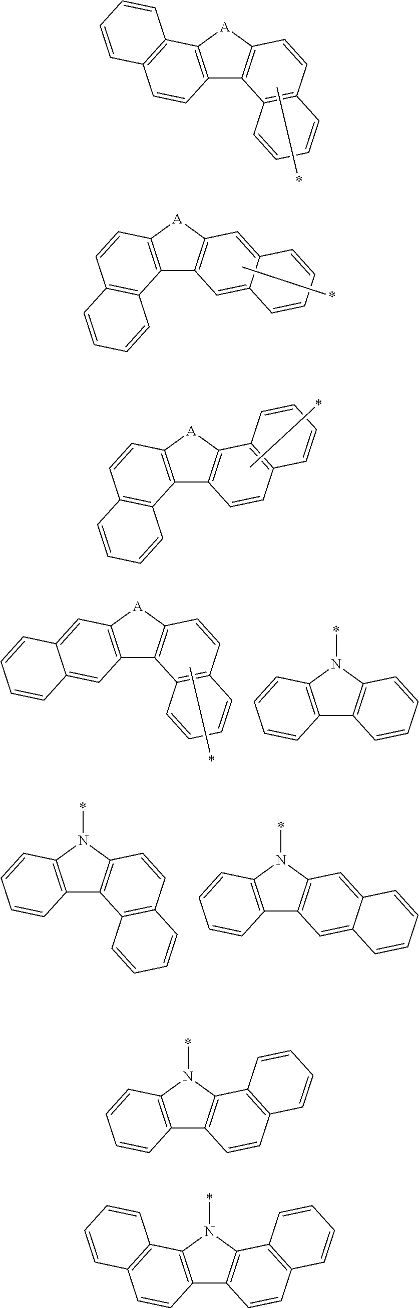 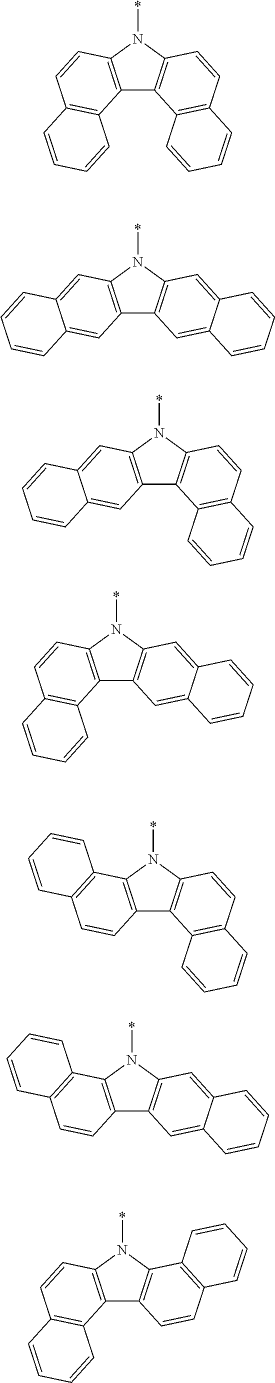 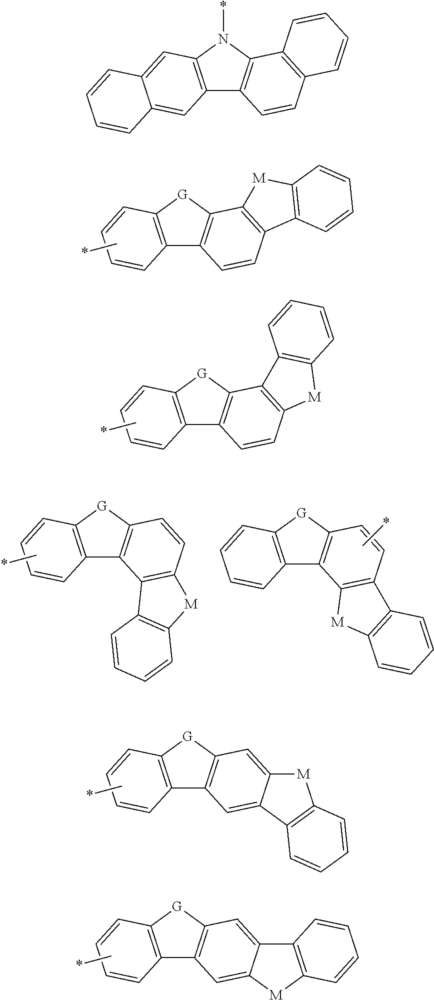 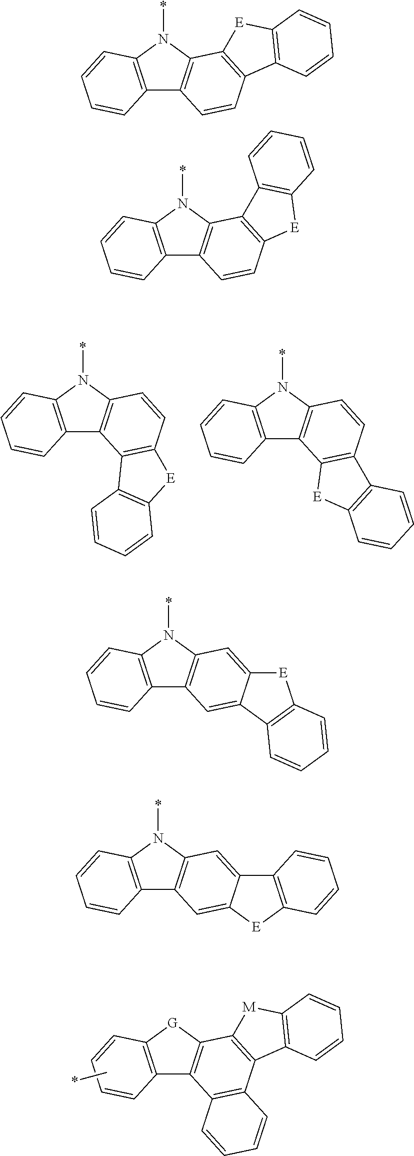 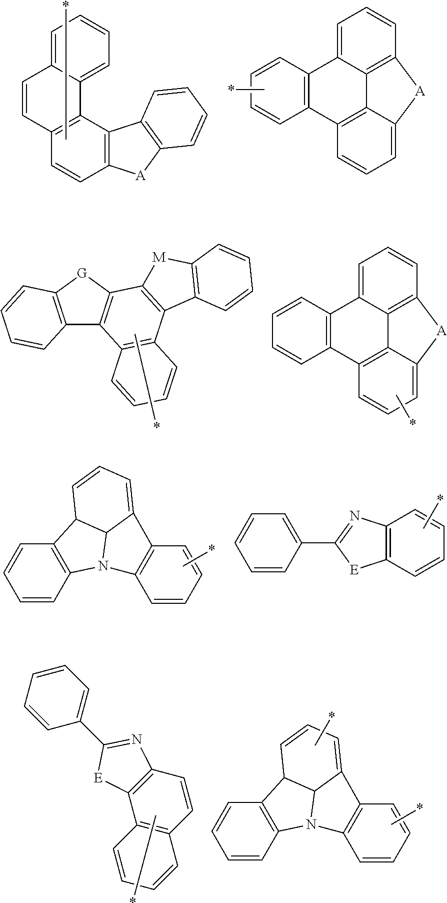
wherein,
A, G, E, and M, each independently, represent O, S, NR₁₀₆, CR₁₀₇R₁₀₈, or SiR₁₀₉R₁₁₀;
R₁₀₆ to R₁₁₀, each independently, represent a substituted or unsubstituted (C1-C30)alkyl, a substituted or unsubstituted (C6-C30)aryl, or a substituted or unsubstituted (3- to 30-membered)heteroaryl; or may be linked to an adjacent substituent to form a ring(s); and
* represents a bonding site with anthracene backbone or L₁.

5. The plurality of light-emitting materials according to claim 1, wherein formula 2 is represented by the following formula 2-1: 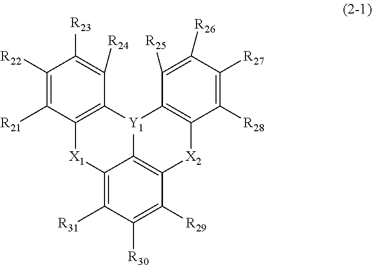
wherein,
Y₁, X₁, and X₂, each are as defined in claim 1; and
R₂₁ to R₃₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a ring(s).

6. The plurality of light-emitting materials according to claim 1, wherein the compound represented by formula 1 is selected from the group consisting of the following compounds:

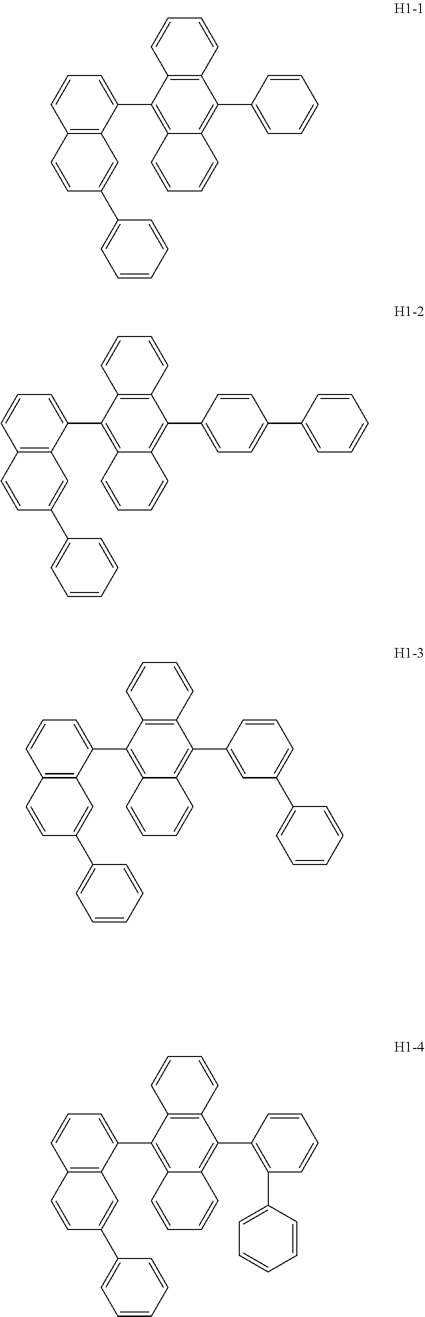

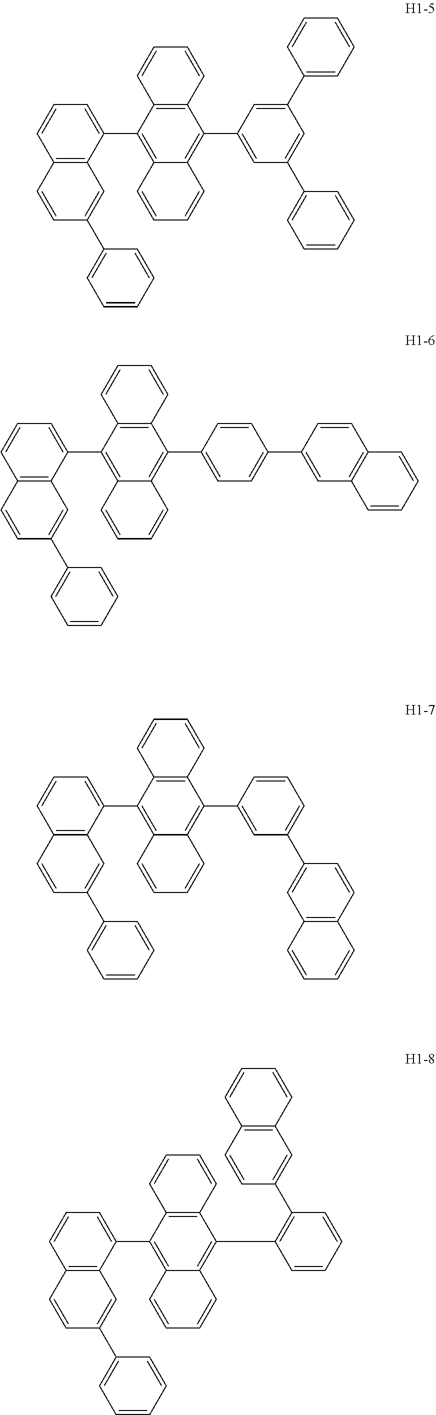

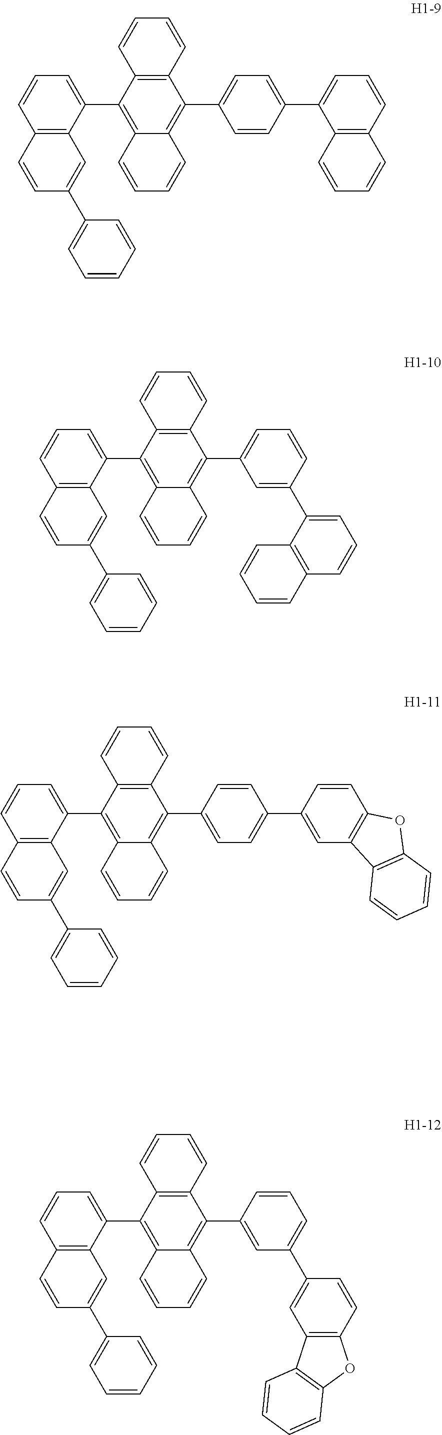

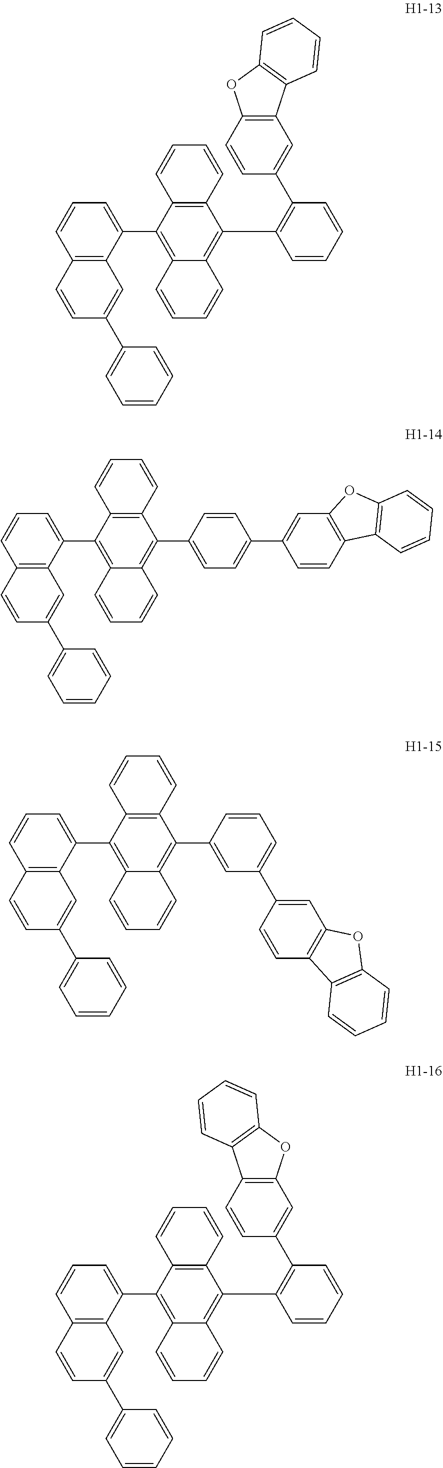 [SMILES: c1ccc(-c2ccc3cccc(-c4c5ccccc5c(-c5ccccc5-c5ccc6oc7ccccc7c6c5)c5ccccc45)c3c2)cc1 ; c1ccc(-c2ccc3cccc(-c4c5ccccc5c(-c5ccc(-c6ccc7c(c6)oc6ccccc67)cc5)c5ccccc45)c3c2)cc1 ; c1ccc(-c2ccc3cccc(-c4c5ccccc5c(-c5cccc(-c6ccc7c(c6)oc6ccccc67)c5)c5ccccc45)c3c2)cc1 ; c1ccc(-c2ccc3cccc(-c4c5ccccc5c(-c5ccccc5-c5ccc6c(c5)oc5ccccc56)c5ccccc45)c3c2)cc1]

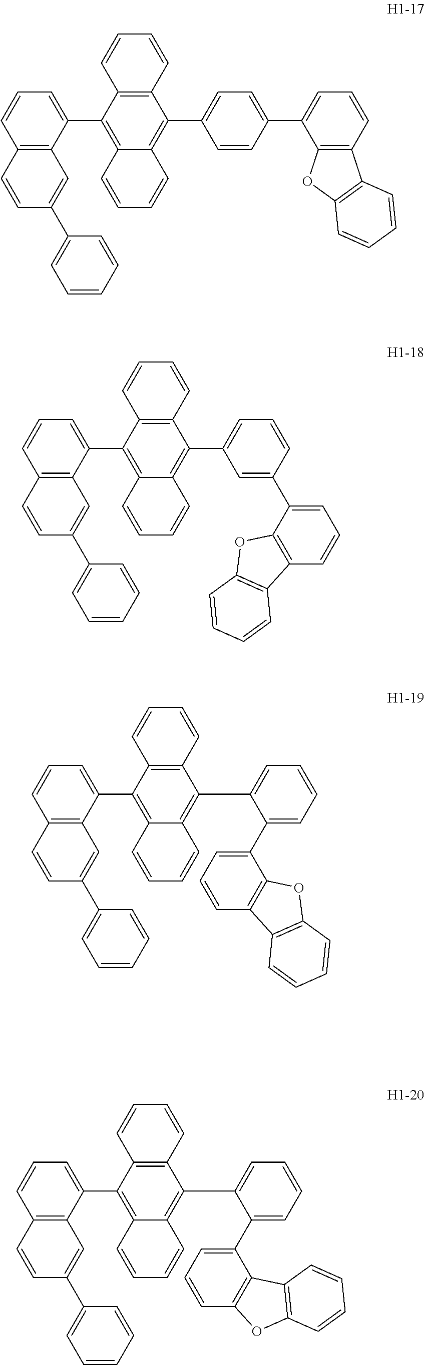 [SMILES: c1ccc(-c2ccc3cccc(-c4c5ccccc5c(-c5ccc(-c6cccc7c6oc6ccccc67)cc5)c5ccccc45)c3c2)cc1 ; c1ccc(-c2ccc3cccc(-c4c5ccccc5c(-c5cccc(-c6cccc7c6oc6ccccc67)c5)c5ccccc45)c3c2)cc1 ; c1ccc(-c2ccc3cccc(-c4c5ccccc5c(-c5ccccc5-c5cccc6c5oc5ccccc56)c5ccccc45)c3c2)cc1 ; c1ccc(-c2ccc3cccc(-c4c5ccccc5c(-c5ccccc5-c5cccc6oc7ccccc7c56)c5ccccc45)c3c2)cc1]

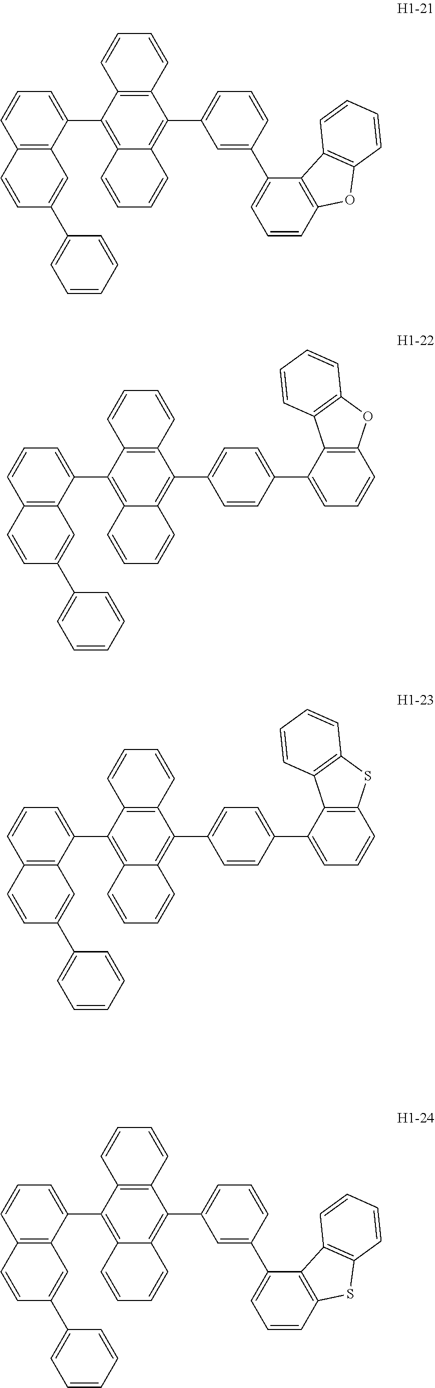 [SMILES: c1ccc(-c2ccc3cccc(-c4c5ccccc5c(-c5cccc(-c6cccc7oc8ccccc8c67)c5)c5ccccc45)c3c2)cc1 ; c1ccc(-c2ccc3cccc(-c4c5ccccc5c(-c5ccc(-c6cccc7oc8ccccc8c67)cc5)c5ccccc45)c3c2)cc1 ; c1ccc(-c2ccc3cccc(-c4c5ccccc5c(-c5ccc(-c6cccc7sc8ccccc8c67)cc5)c5ccccc45)c3c2)cc1 ; c1ccc(-c2ccc3cccc(-c4c5ccccc5c(-c5cccc(-c6cccc7sc8ccccc8c67)c5)c5ccccc45)c3c2)cc1]

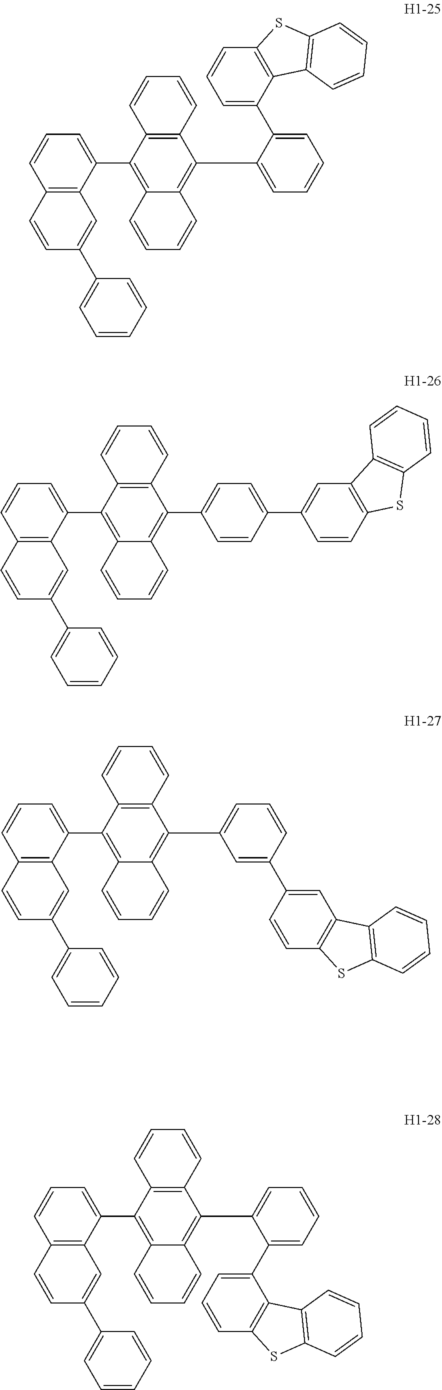 [SMILES: c1ccc(-c2ccc3cccc(-c4c5ccccc5c(-c5ccccc5-c5cccc6sc7ccccc7c56)c5ccccc45)c3c2)cc1 ; c1ccc(-c2ccc3cccc(-c4c5ccccc5c(-c5ccc(-c6ccc7sc8ccccc8c7c6)cc5)c5ccccc45)c3c2)cc1 ; c1ccc(-c2ccc3cccc(-c4c5ccccc5c(-c5cccc(-c6ccc7sc8ccccc8c7c6)c5)c5ccccc45)c3c2)cc1 ; c1ccc(-c2ccc3cccc(-c4c5ccccc5c(-c5ccccc5-c5cccc6sc7ccccc7c56)c5ccccc45)c3c2)cc1]

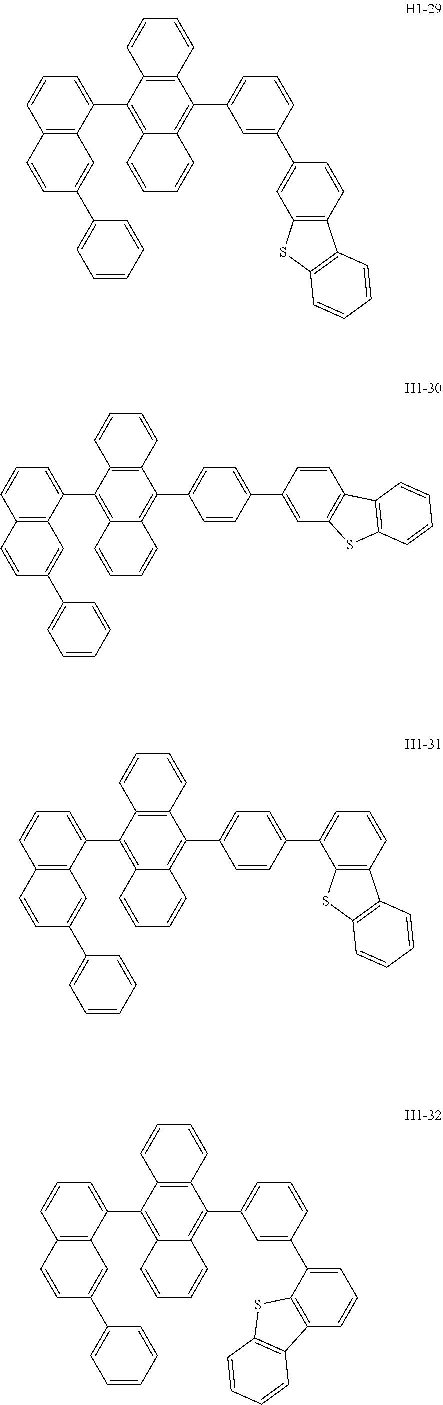 [SMILES: c1ccc(-c2ccc3cccc(-c4c5ccccc5c(-c5cccc(-c6ccc7c(c6)sc6ccccc67)c5)c5ccccc45)c3c2)cc1 ; c1ccc(-c2ccc3cccc(-c4c5ccccc5c(-c5ccc(-c6ccc7c(c6)sc6ccccc67)cc5)c5ccccc45)c3c2)cc1 ; c1ccc(-c2ccc3cccc(-c4c5ccccc5c(-c5ccc(-c6cccc7c6sc6ccccc67)cc5)c5ccccc45)c3c2)cc1 ; c1ccc(-c2ccc3cccc(-c4c5ccccc5c(-c5cccc(-c6cccc7c6sc6ccccc67)c5)c5ccccc45)c3c2)cc1]

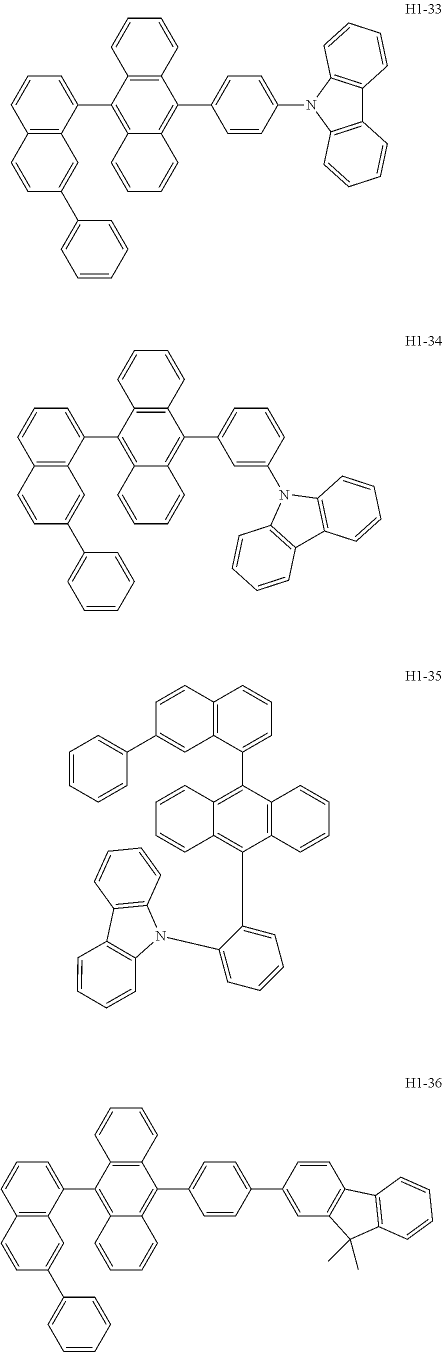

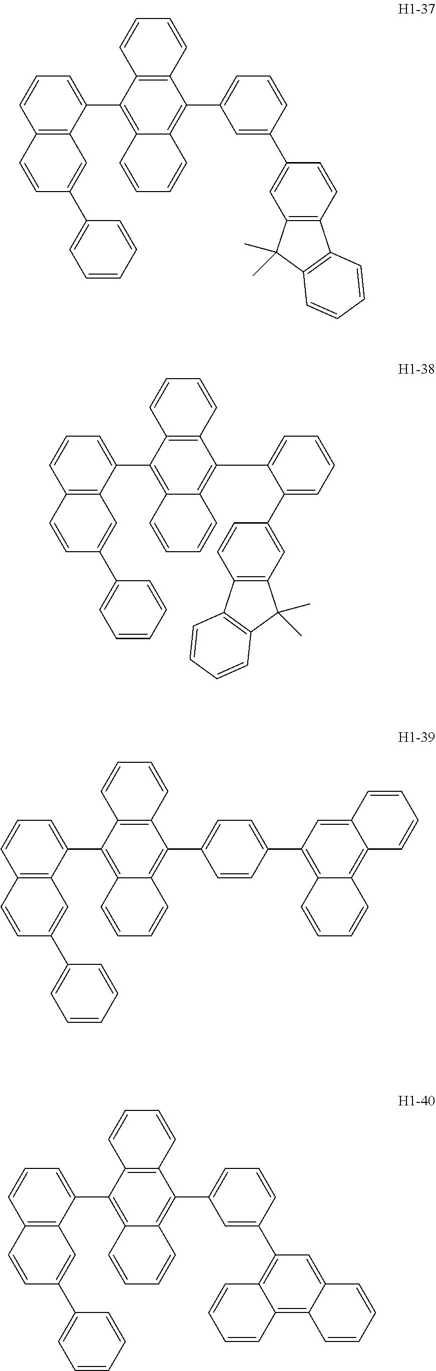

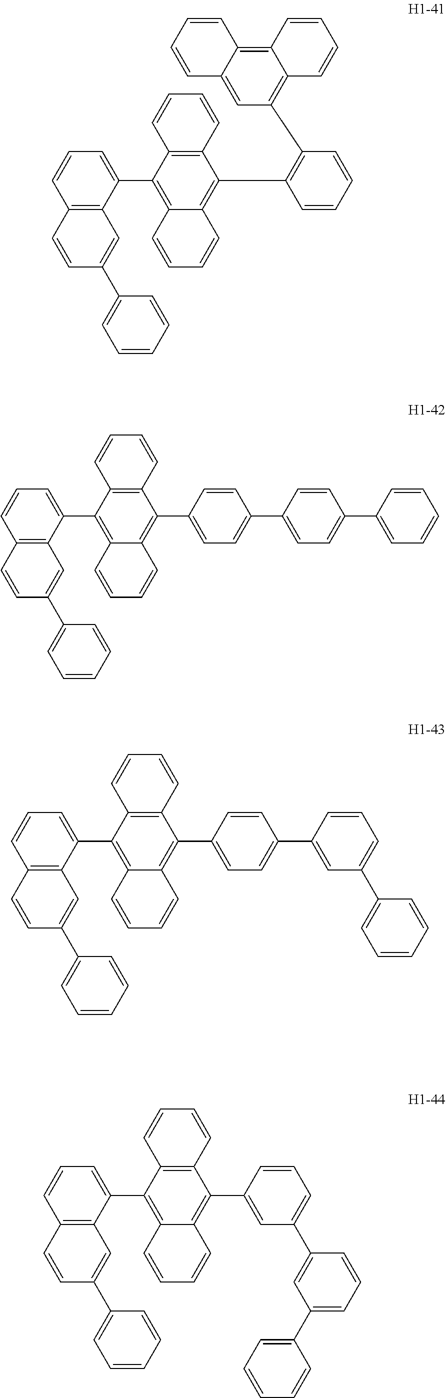

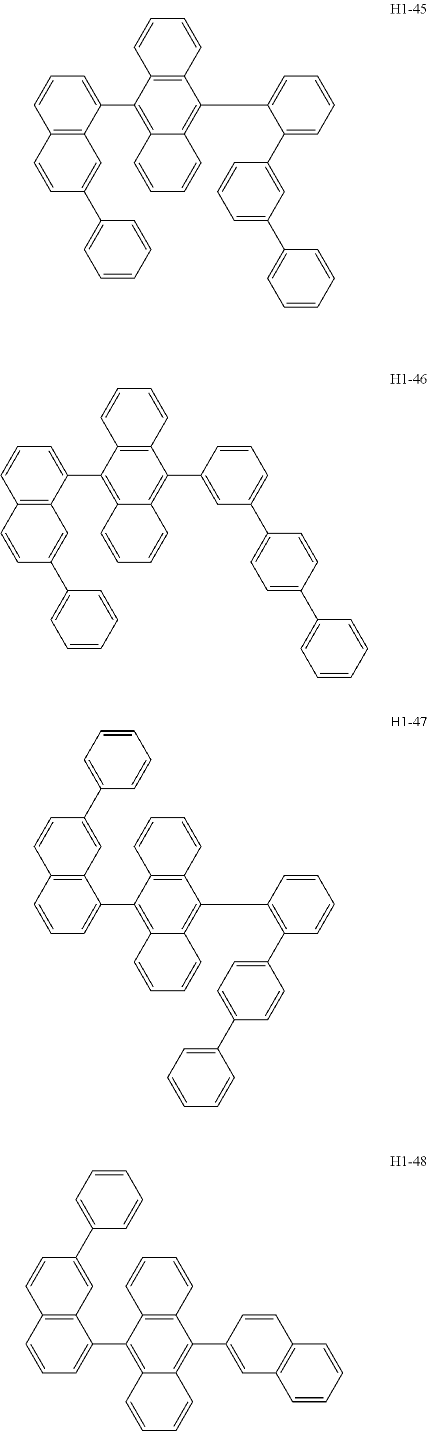

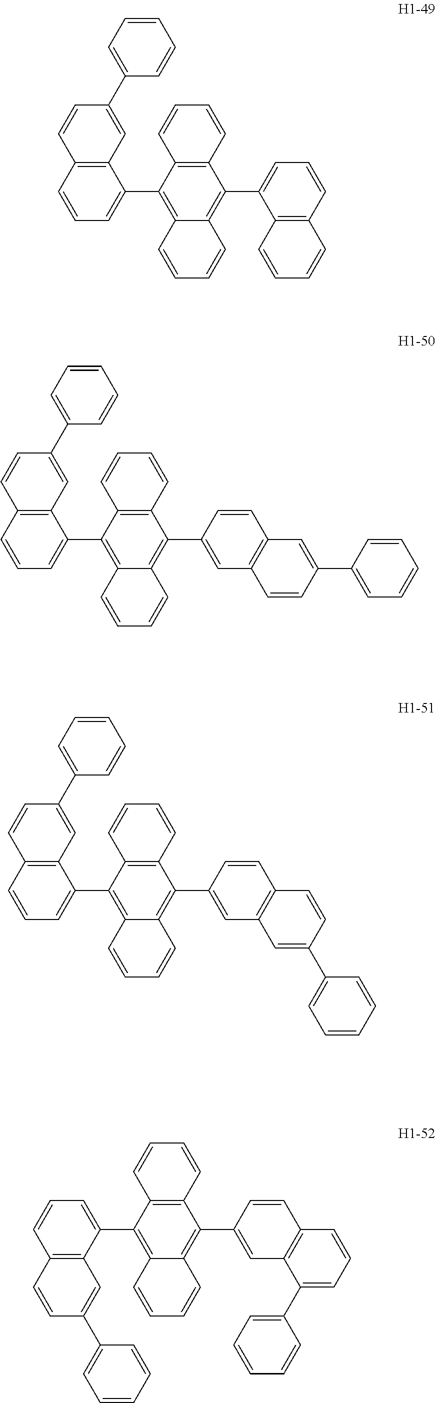

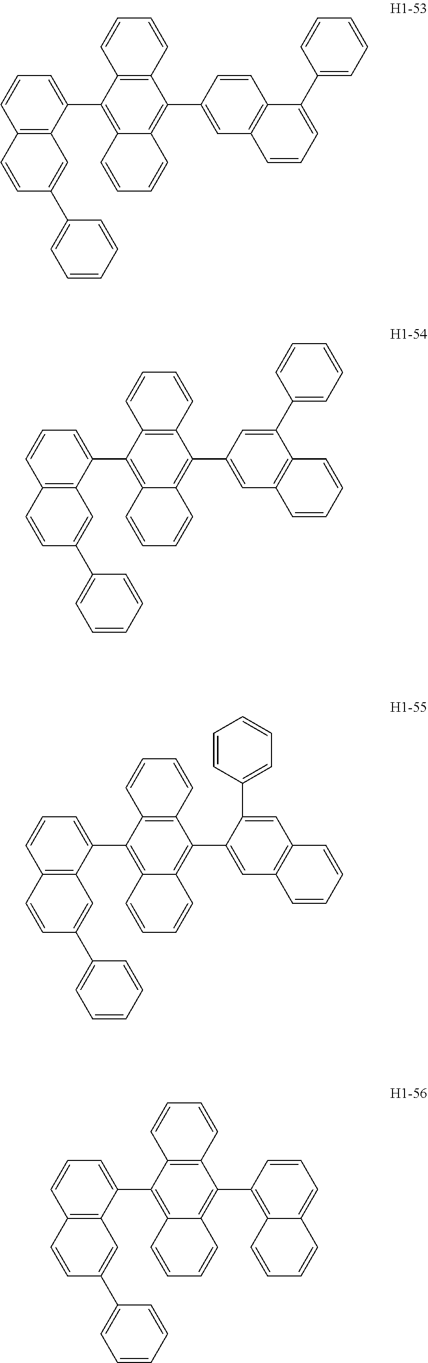

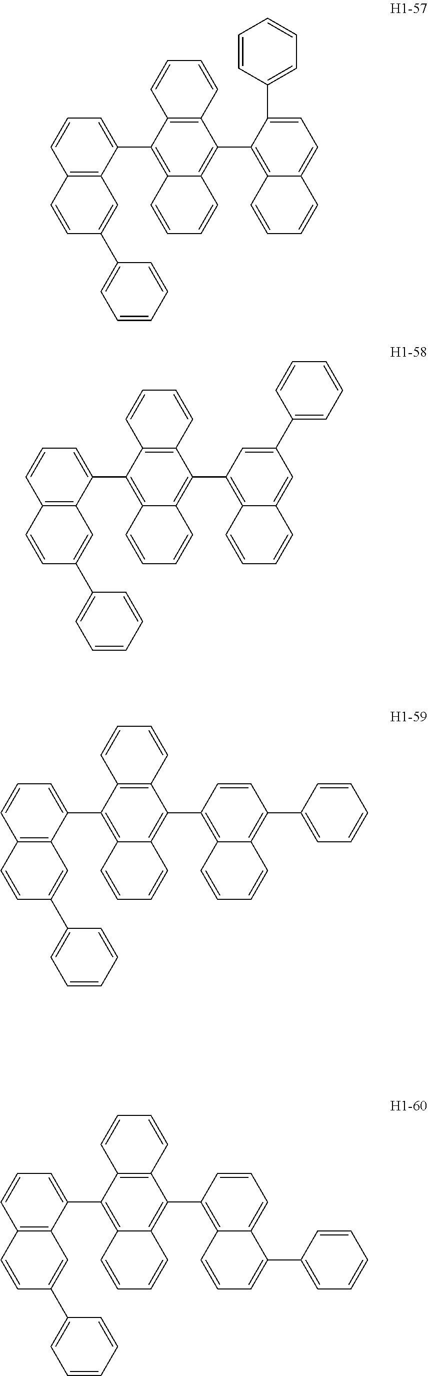

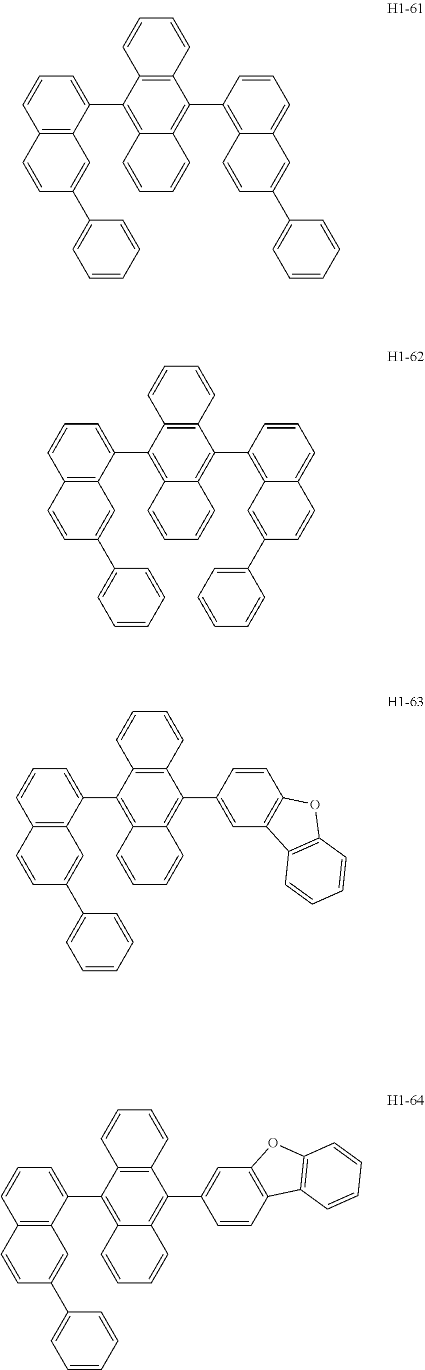

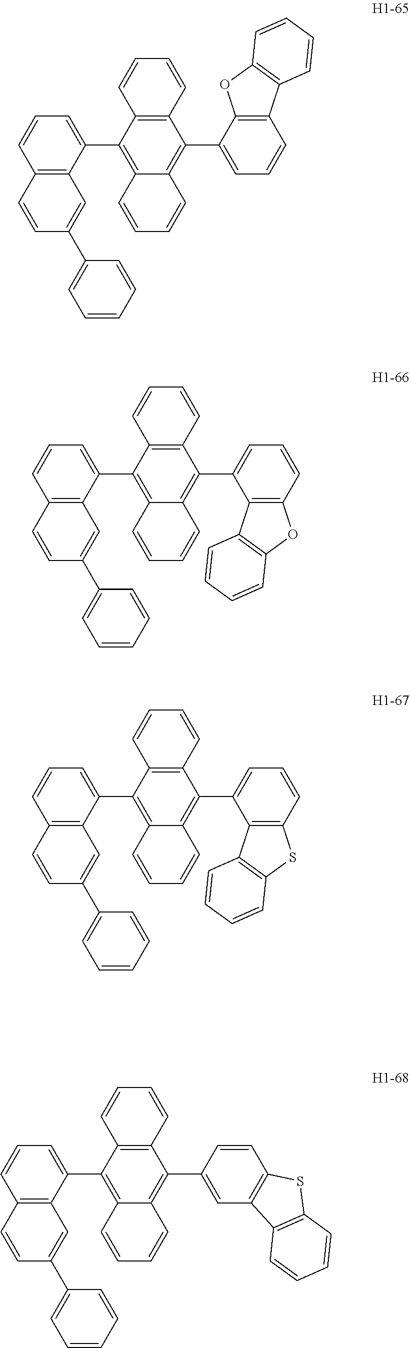 [SMILES: c1ccc(-c2ccc3cccc(-c4c5ccccc5c(-c5cccc6c5oc5ccccc56)c5ccccc45)c3c2)cc1 ; c1ccc(-c2ccc3cccc(-c4c5ccccc5c(-c5cccc6oc7ccccc7c56)c5ccccc45)c3c2)cc1 ; c1ccc(-c2ccc3cccc(-c4c5ccccc5c(-c5cccc6sc7ccccc7c56)c5ccccc45)c3c2)cc1 ; c1ccc(-c2ccc3cccc(-c4c5ccccc5c(-c5ccc6sc7ccccc7c6c5)c5ccccc45)c3c2)cc1]

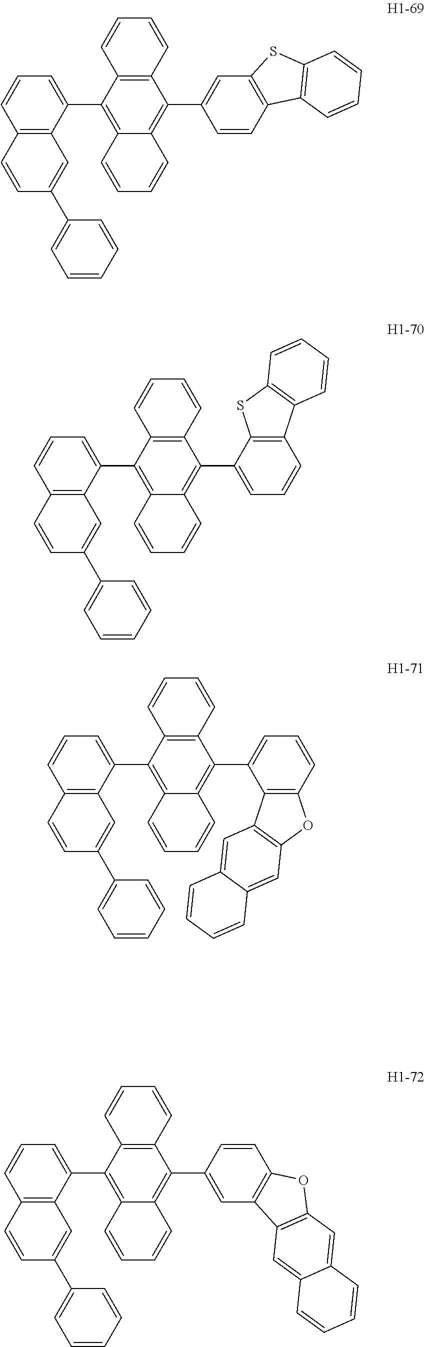 [SMILES: c1ccc(-c2ccc3cccc(-c4c5ccccc5c(-c5ccc6c(c5)sc5ccccc56)c5ccccc45)c3c2)cc1 ; c1ccc(-c2ccc3cccc(-c4c5ccccc5c(-c5cccc6c5sc5ccccc56)c5ccccc45)c3c2)cc1 ; c1ccc(-c2ccc3cccc(-c4c5ccccc5c(-c5cccc6oc7cc8ccccc8cc7c56)c5ccccc45)c3c2)cc1 ; c1ccc(-c2ccc3cccc(-c4c5ccccc5c(-c5ccc6oc7cc8ccccc8cc7c6c5)c5ccccc45)c3c2)cc1]

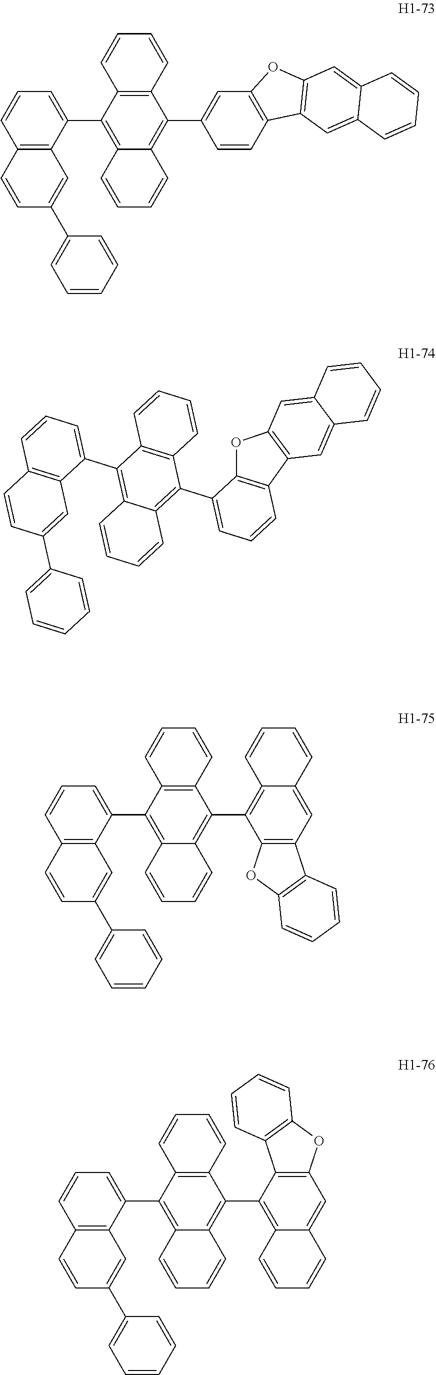 [SMILES: c1ccc(-c2ccc3cccc(-c4c5ccccc5c(-c5ccc6c(c5)oc5cc7ccccc7cc56)c5ccccc45)c3c2)cc1 ; c1ccc(-c2ccc3cccc(-c4c5ccccc5c(-c5cccc6c5oc5cc7ccccc7cc56)c5ccccc45)c3c2)cc1 ; c1ccc(-c2ccc3cccc(-c4c5ccccc5c(-c5c6ccccc6cc6c5oc5ccccc56)c5ccccc45)c3c2)cc1 ; c1ccc(-c2ccc3cccc(-c4c5ccccc5c(-c5c6ccccc6cc6oc7ccccc7c56)c5ccccc45)c3c2)cc1]

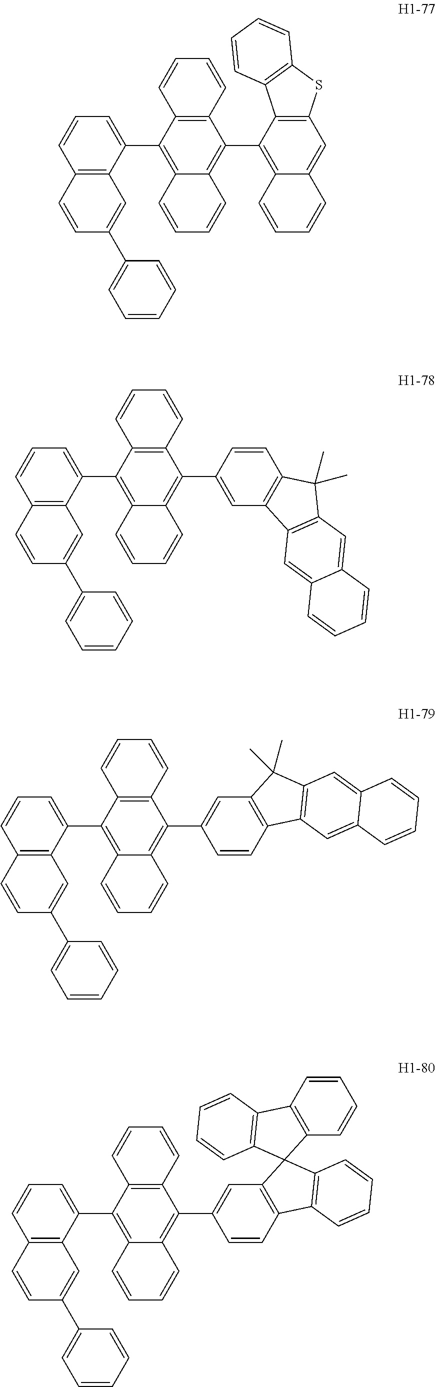

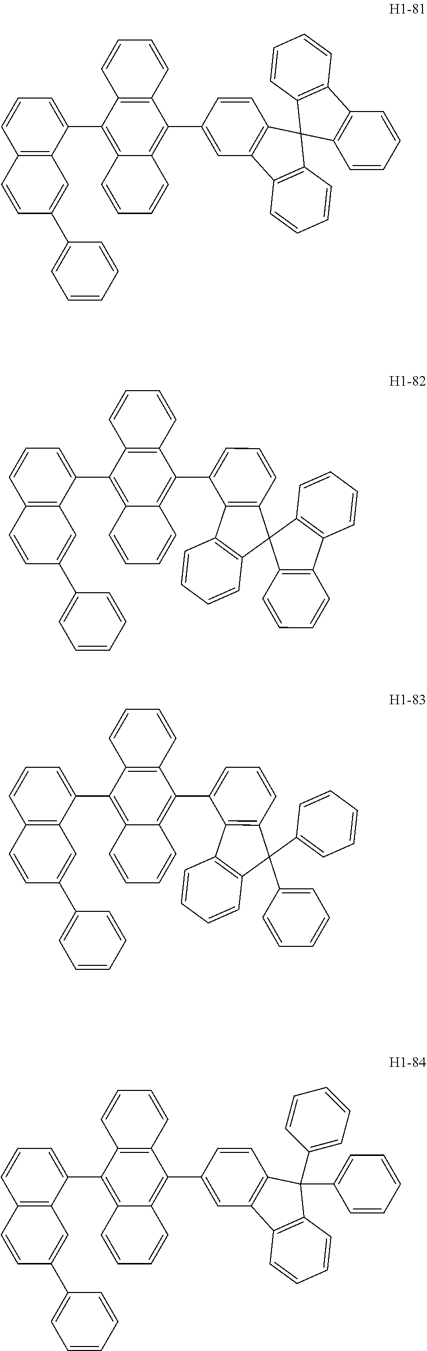

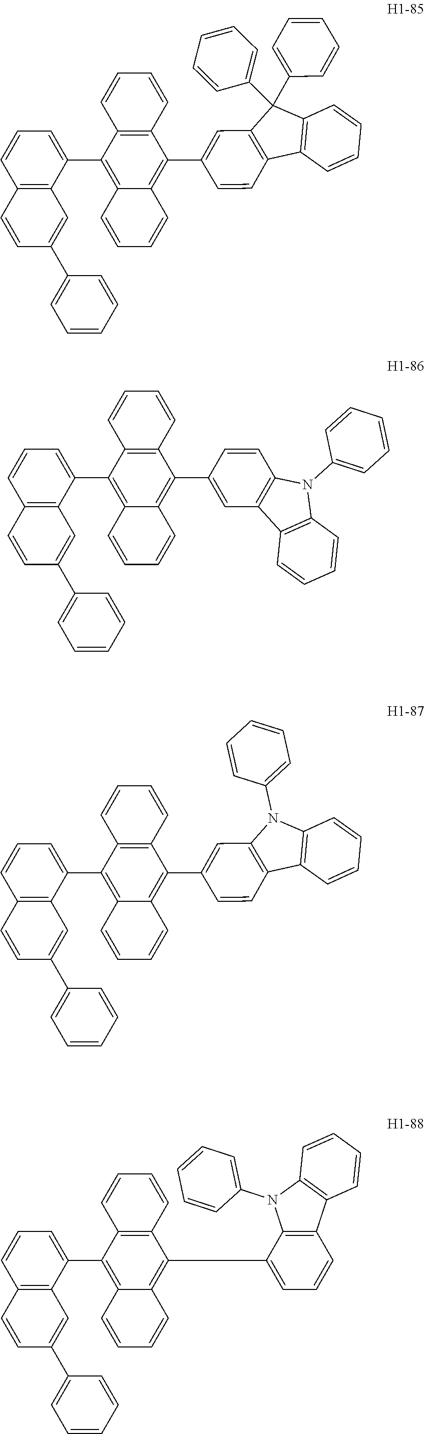

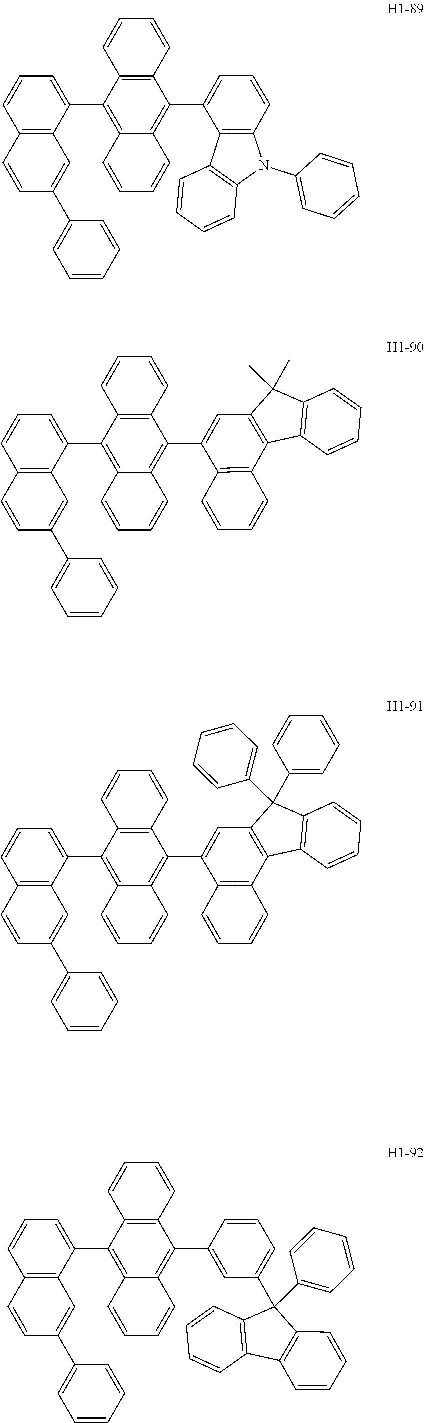

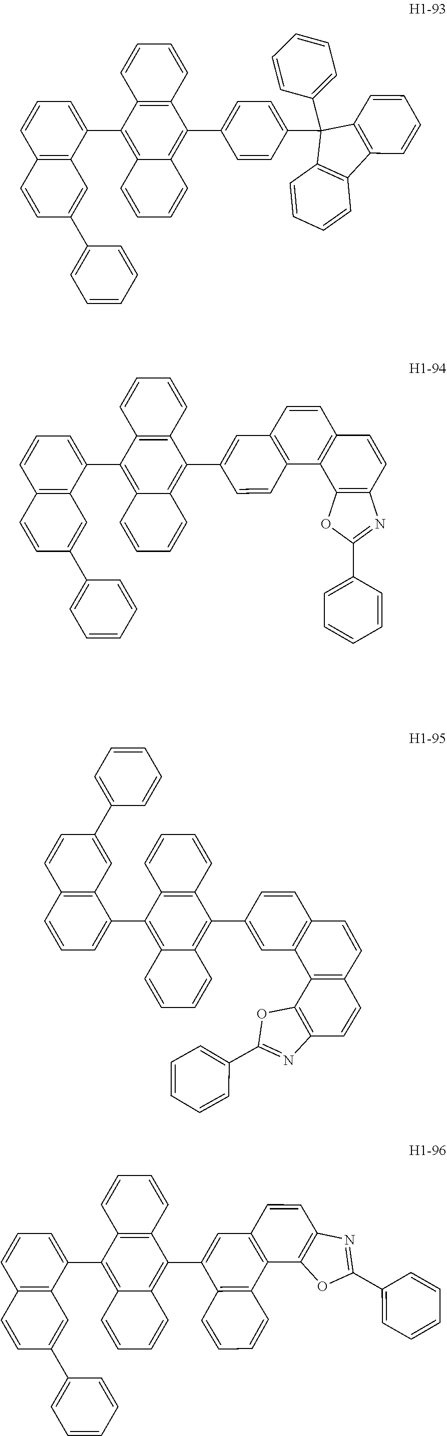

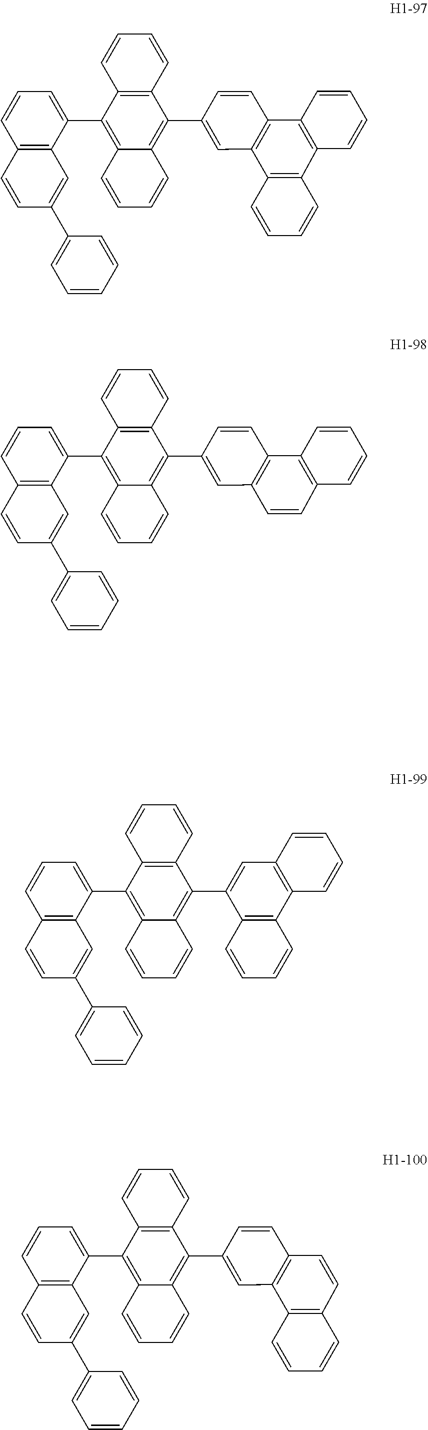

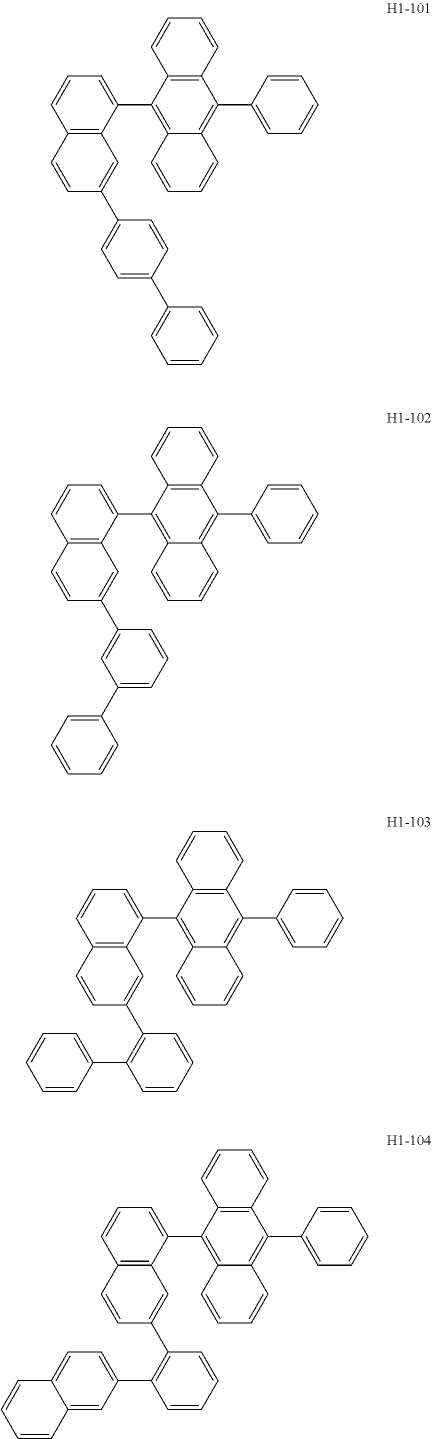

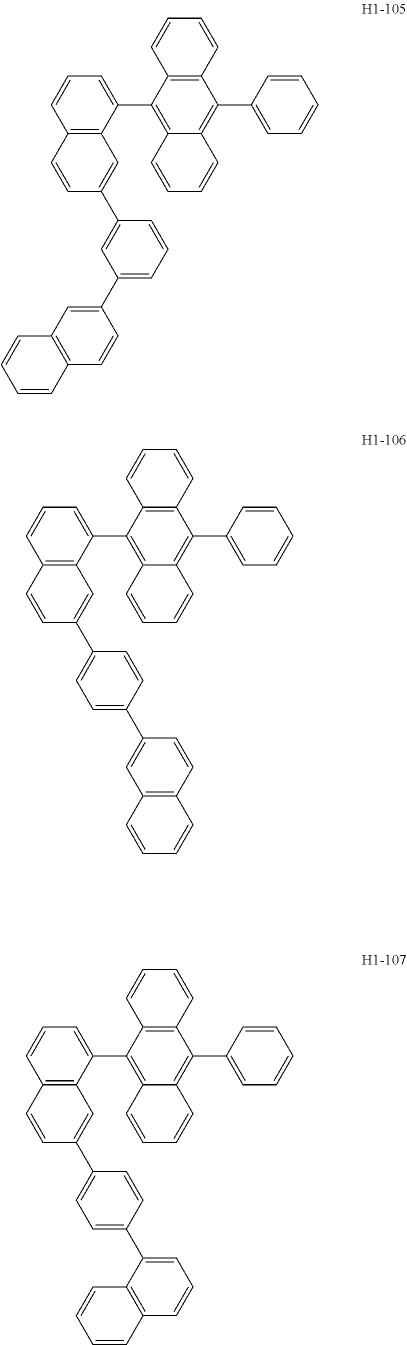

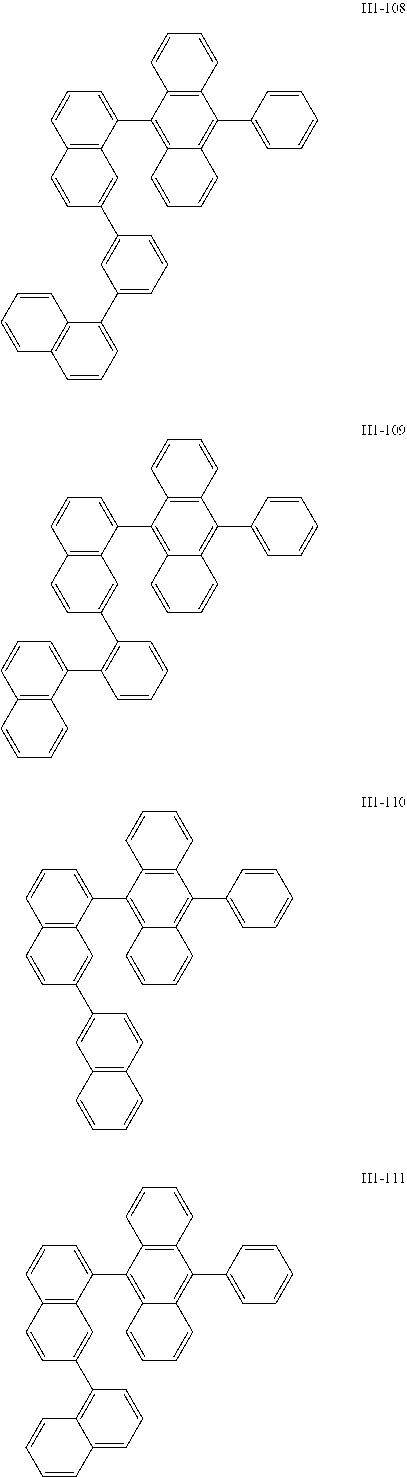

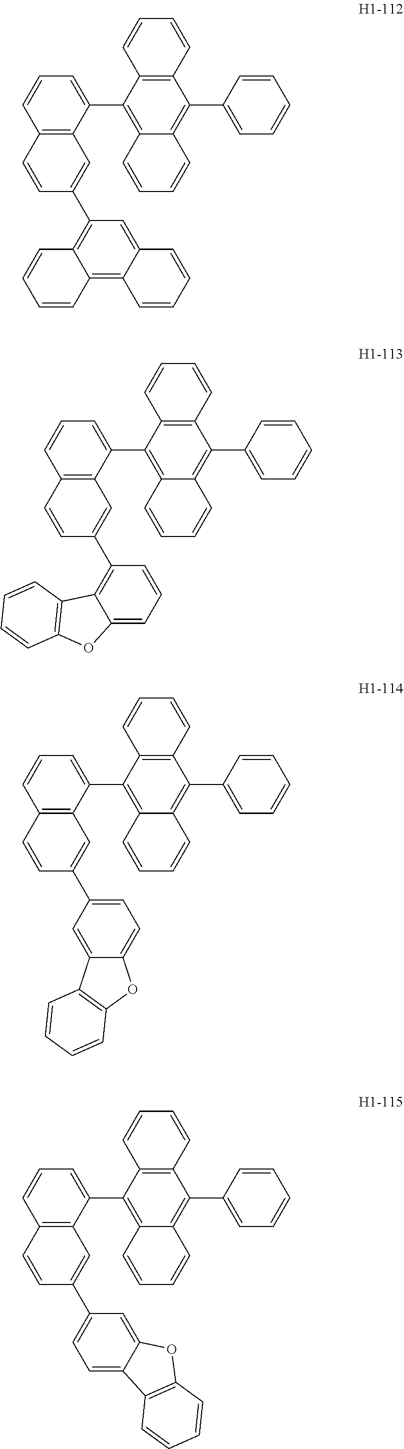

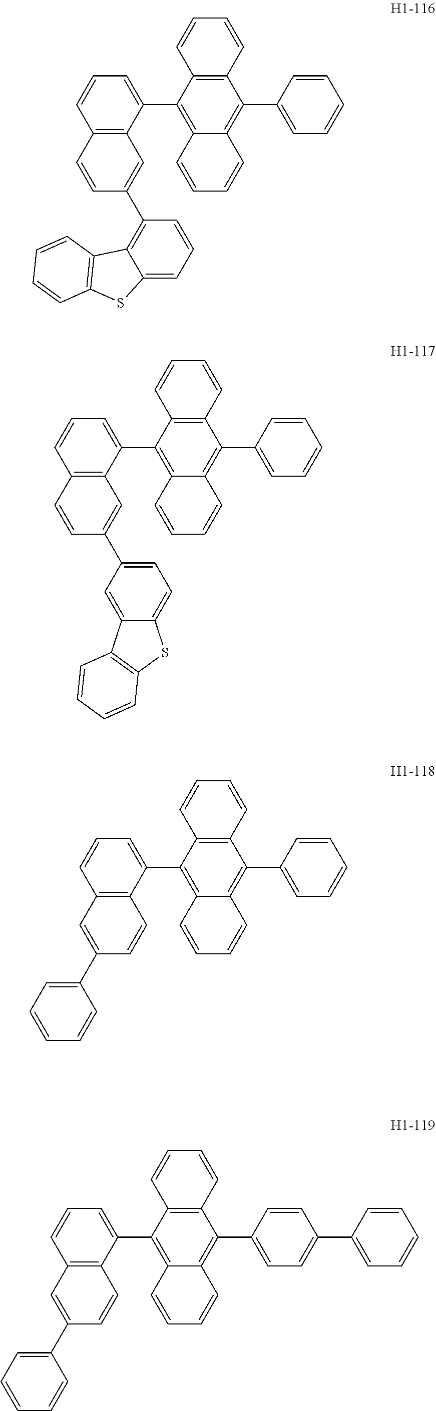

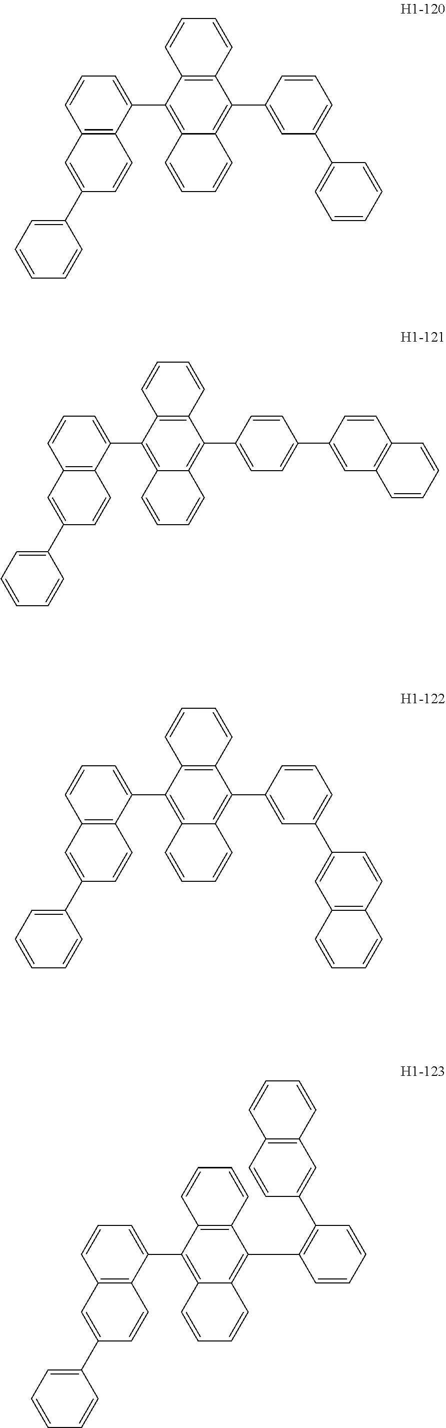

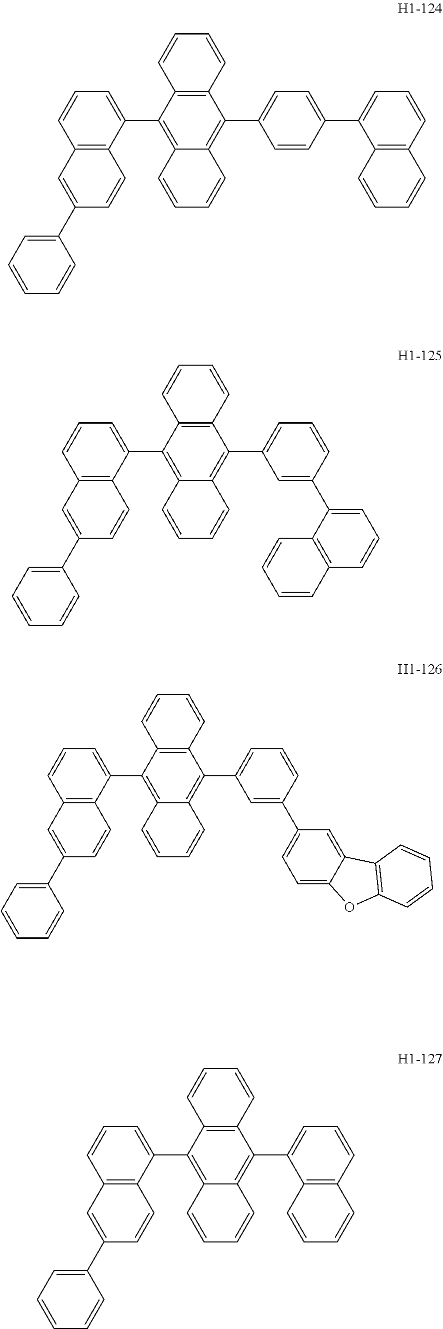

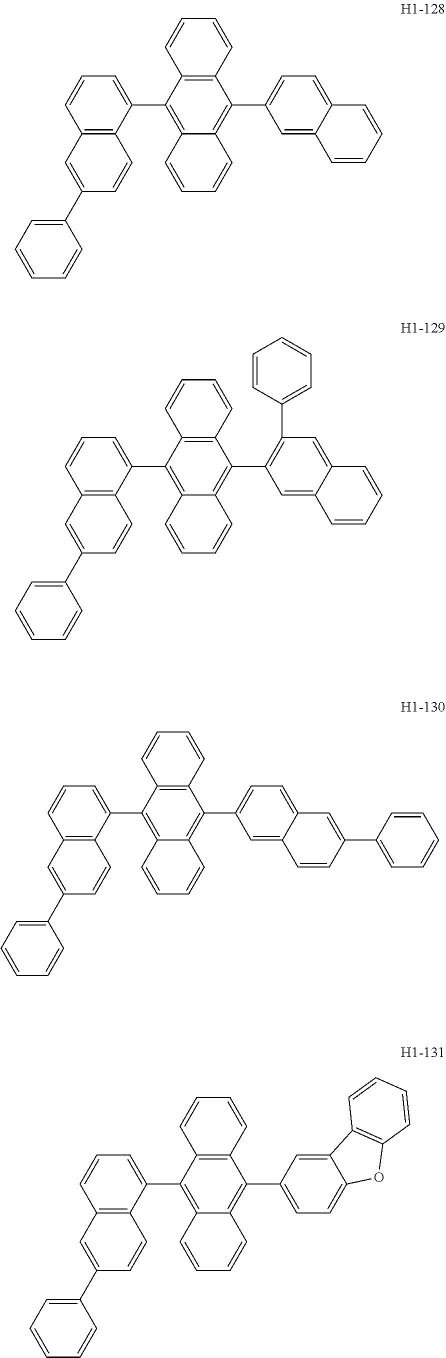

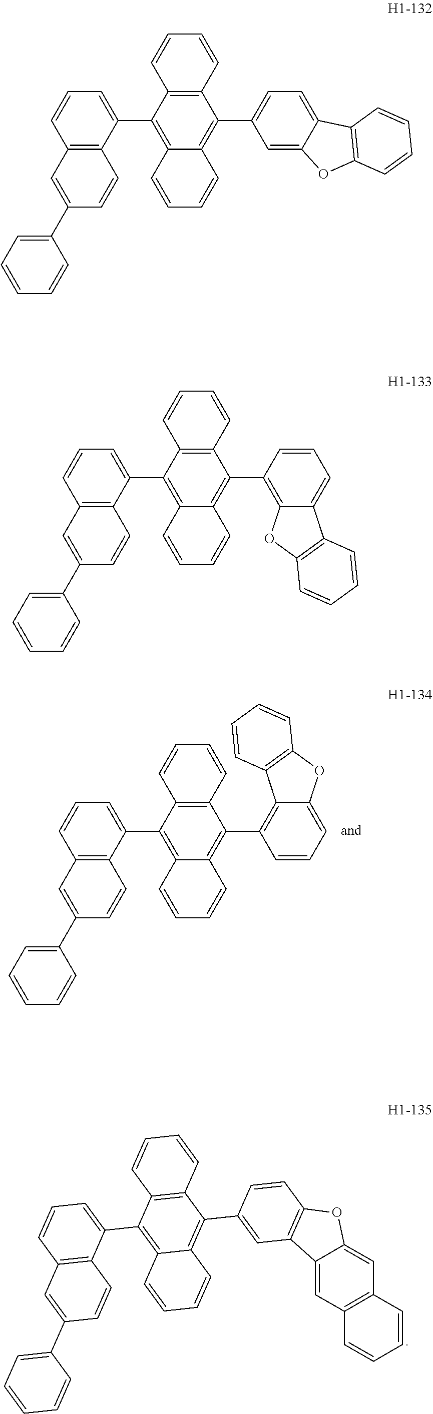 [SMILES: c1ccc(-c2ccc3c(-c4c5ccccc5c(-c5ccc6c(c5)oc5ccccc56)c5ccccc45)cccc3c2)cc1 ; c1ccc(-c2ccc3c(-c4c5ccccc5c(-c5cccc6c5oc5ccccc56)c5ccccc45)cccc3c2)cc1 ; c1ccc(-c2ccc3c(-c4c5ccccc5c(-c5cccc6oc7ccccc7c56)c5ccccc45)cccc3c2)cc1 ; c1ccc(-c2ccc3c(-c4c5ccccc5c(-c5ccc6oc7cc8ccccc8cc7c6c5)c5ccccc45)cccc3c2)cc1]

7. The plurality of light-emitting materials according to claim 1, wherein the compound represented by formula 2 is selected from the group consisting of the following compounds: 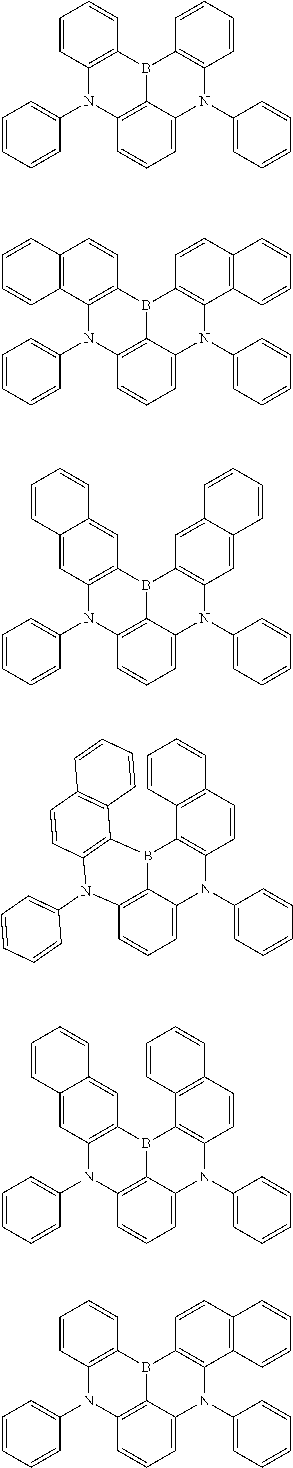 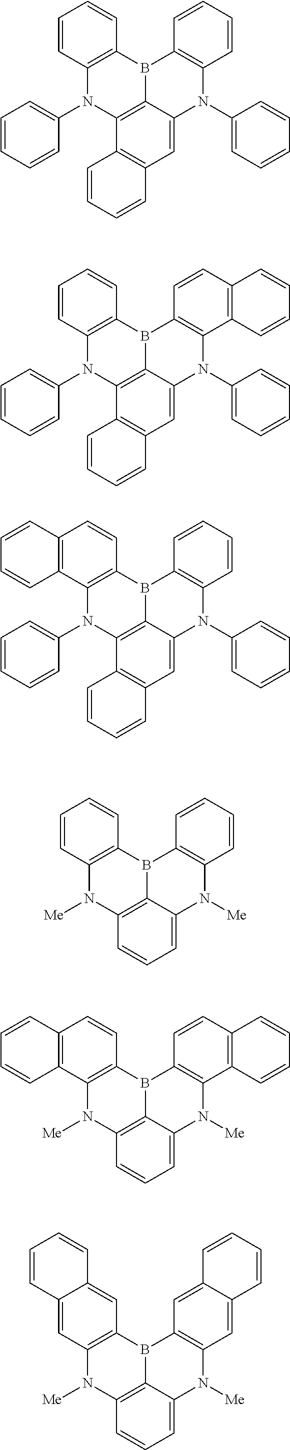 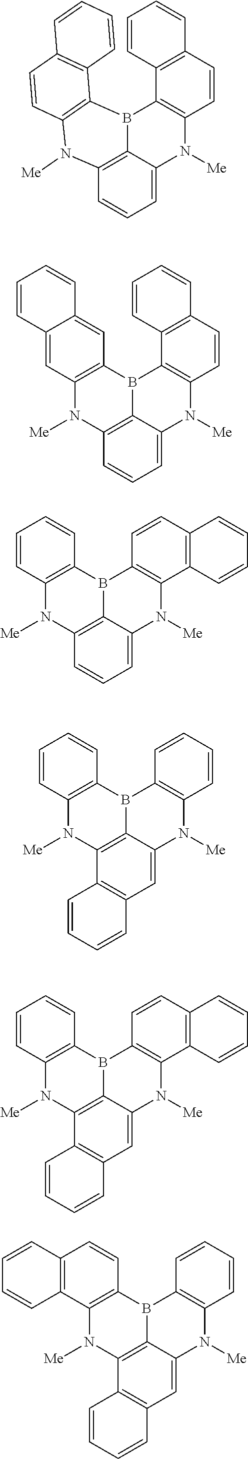 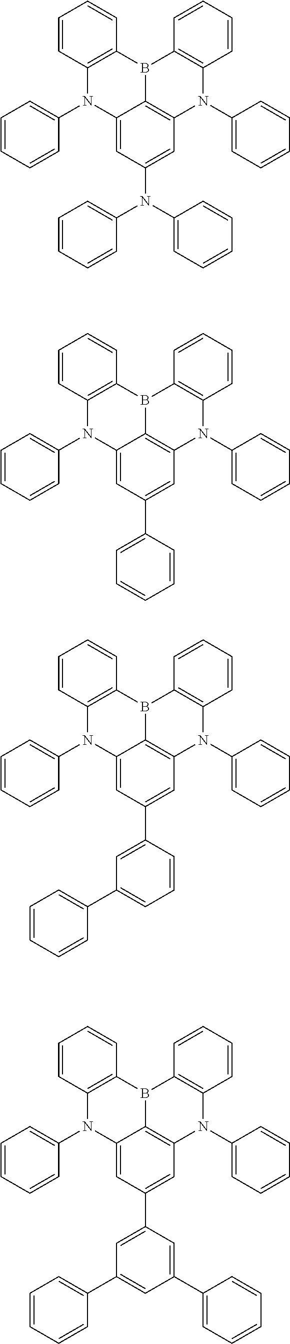 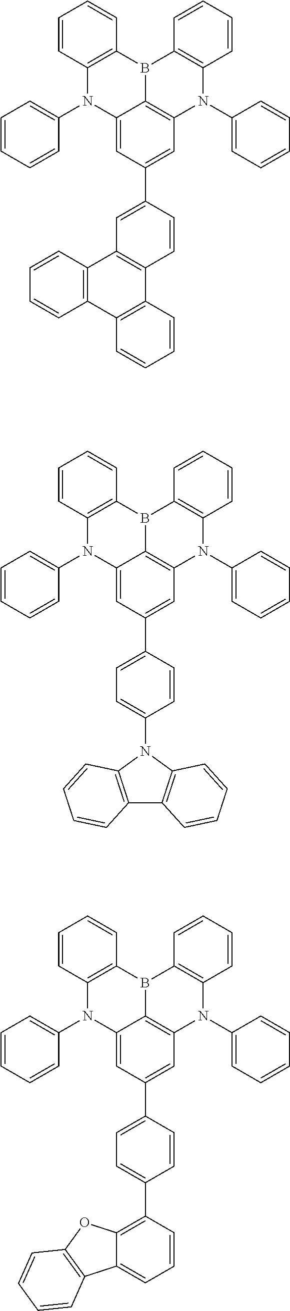 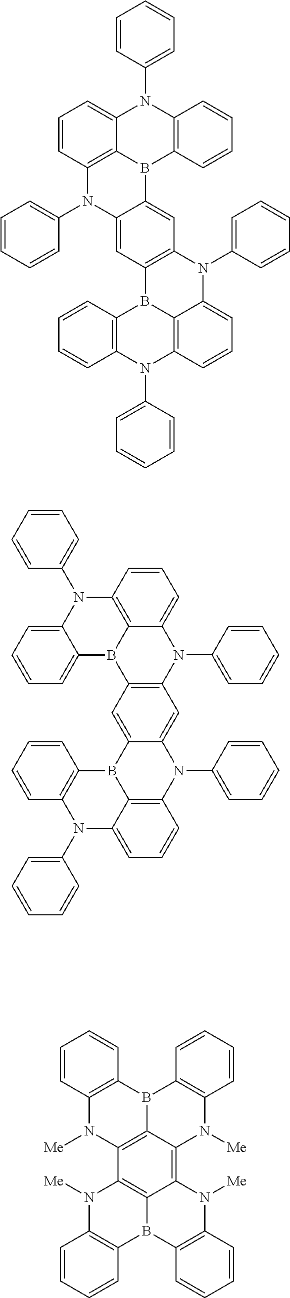 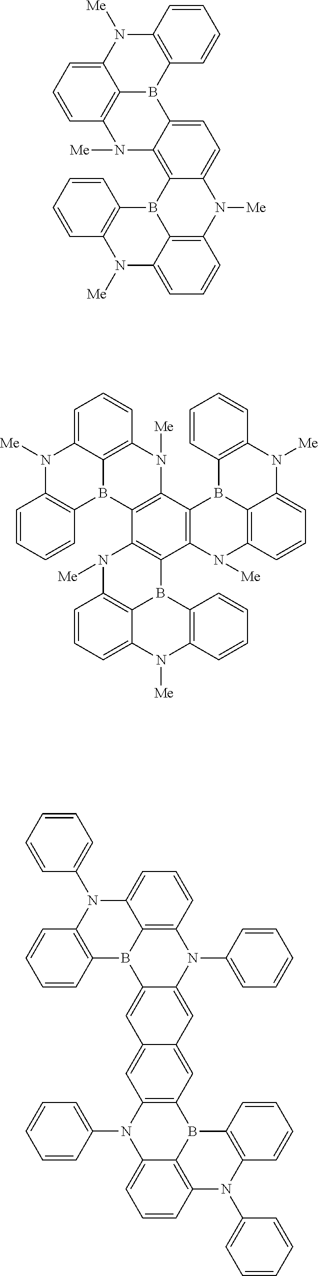 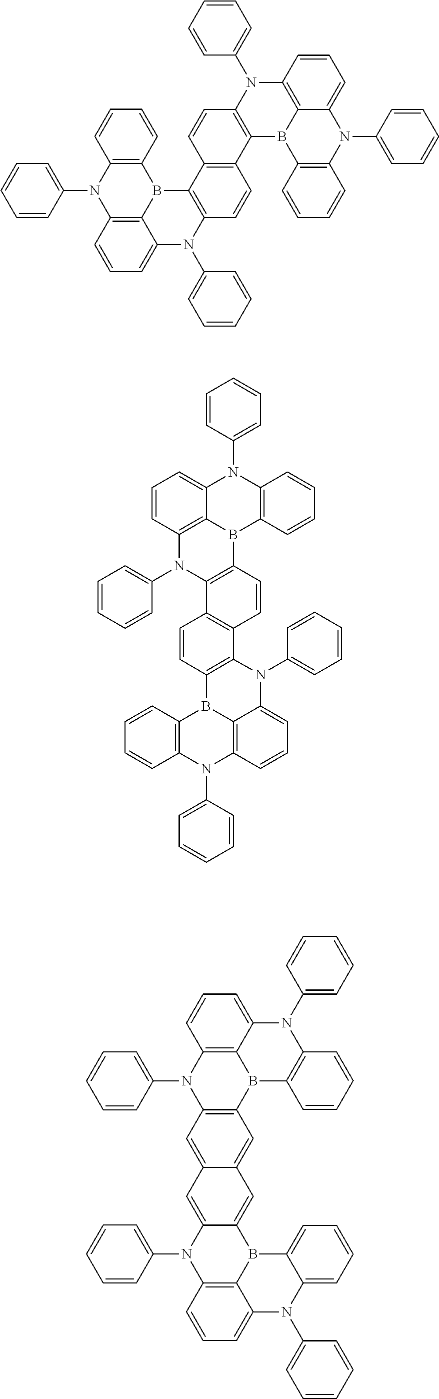 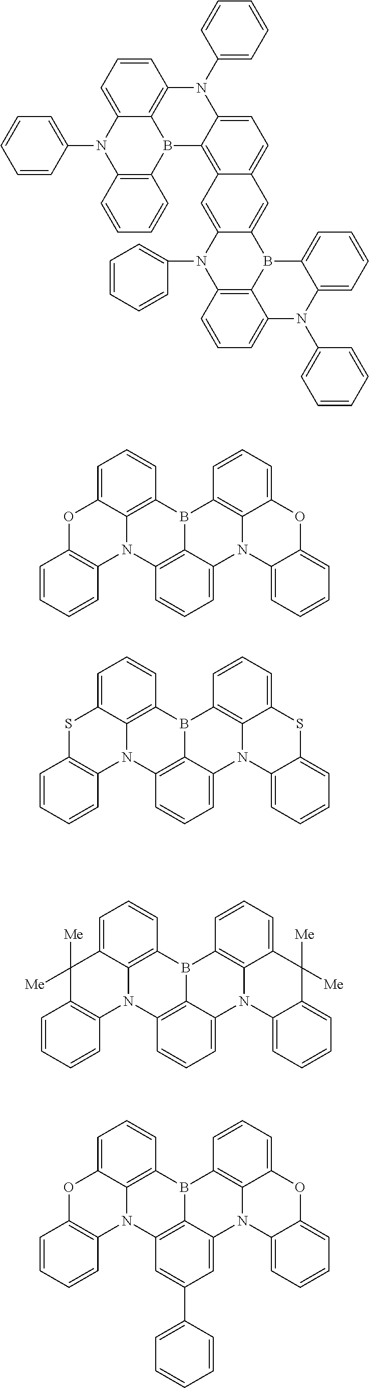 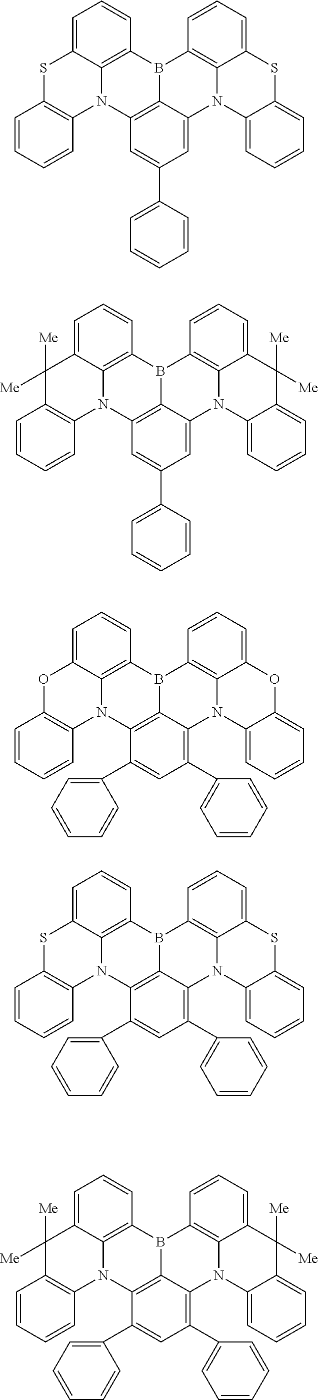 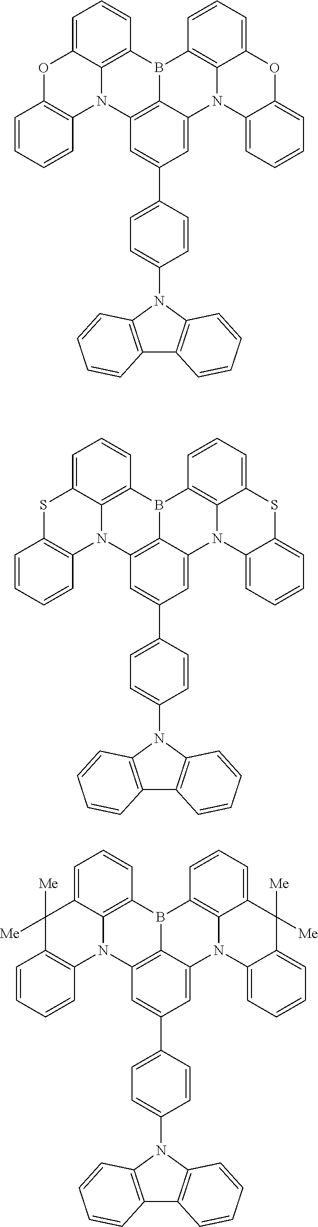 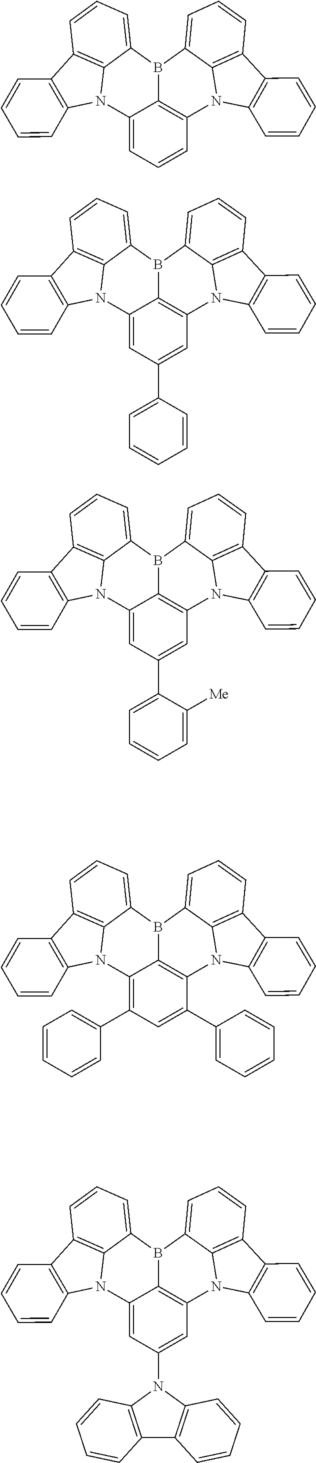 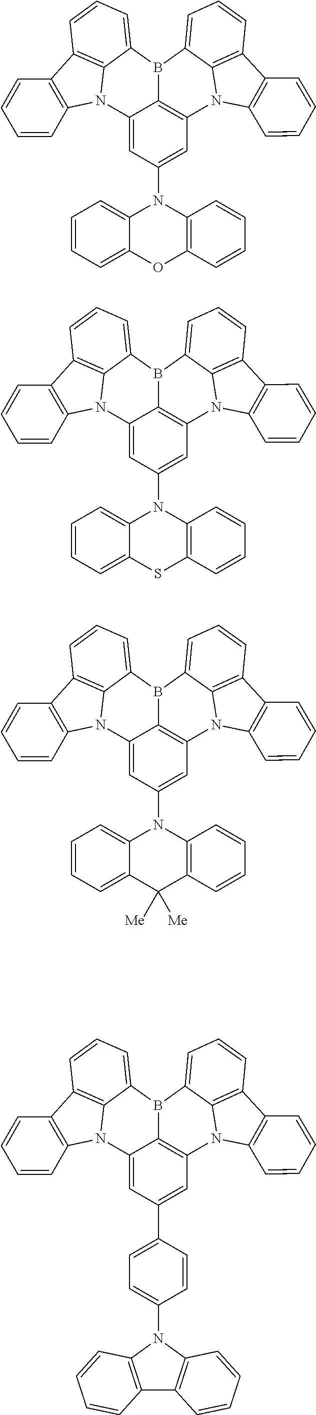 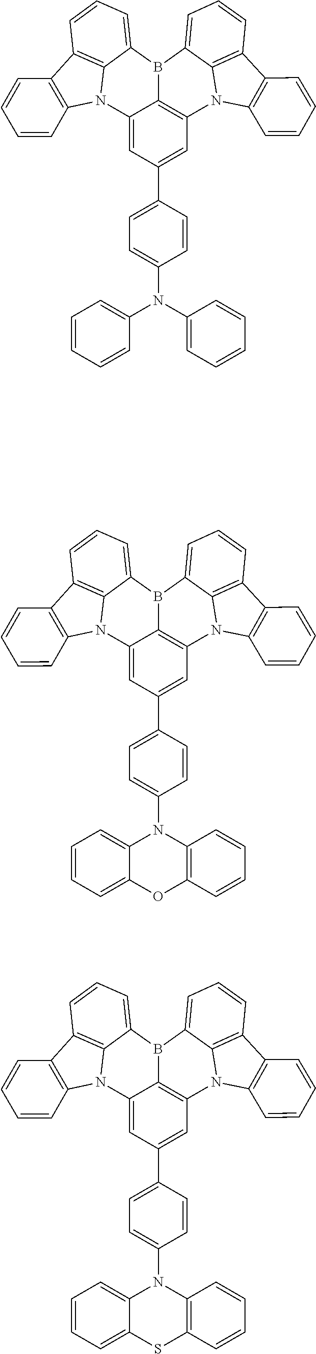 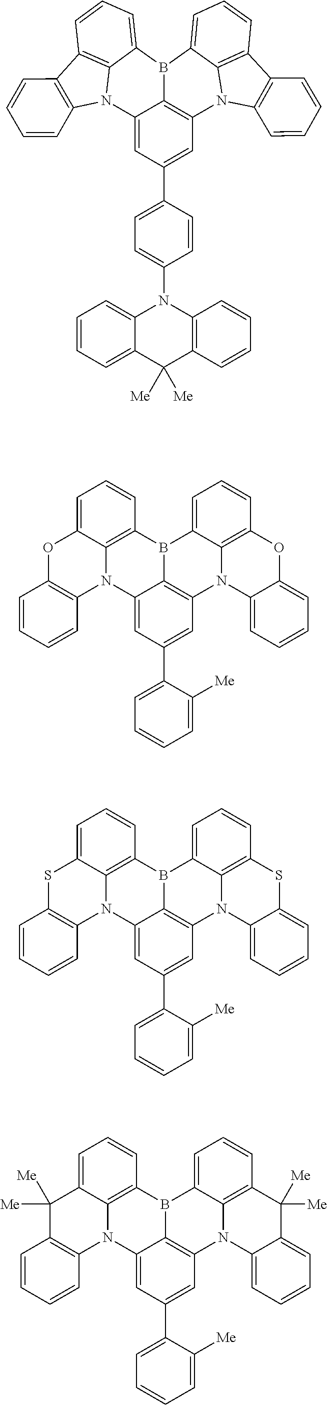 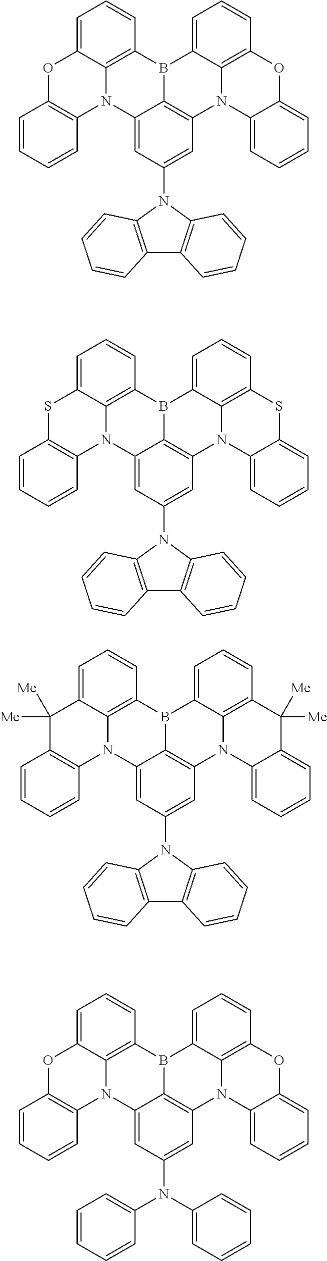 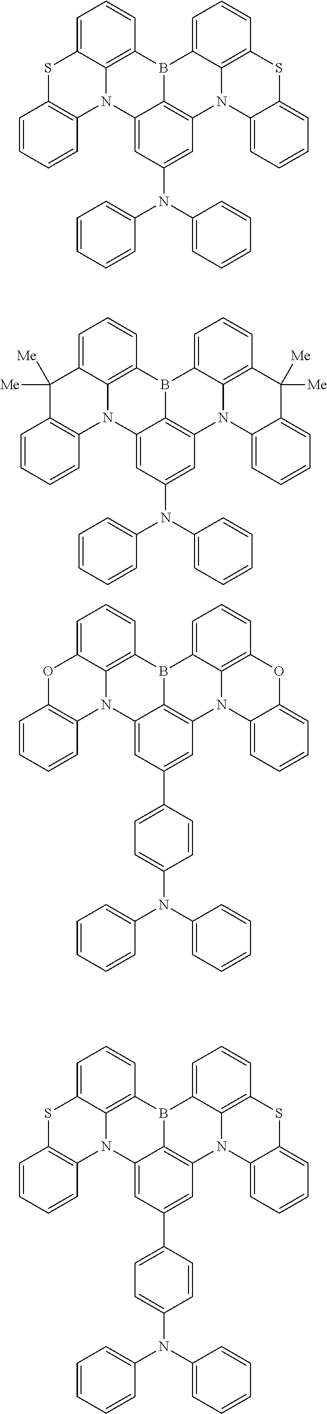 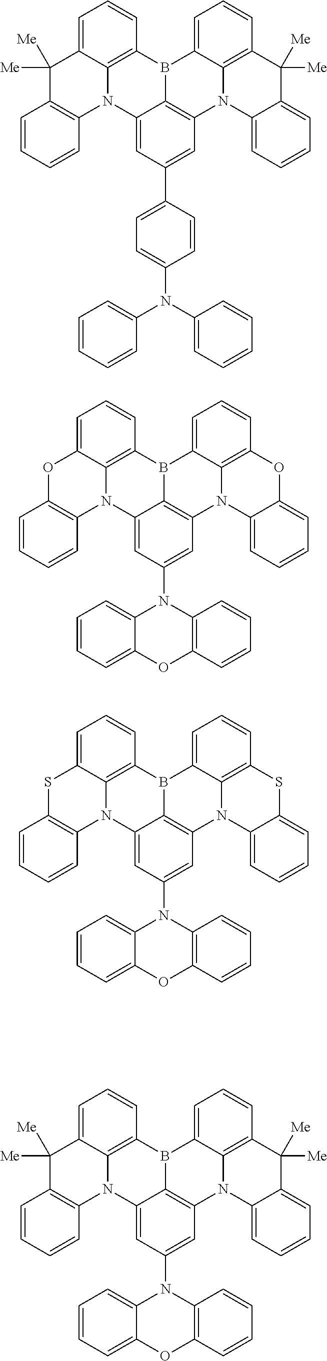 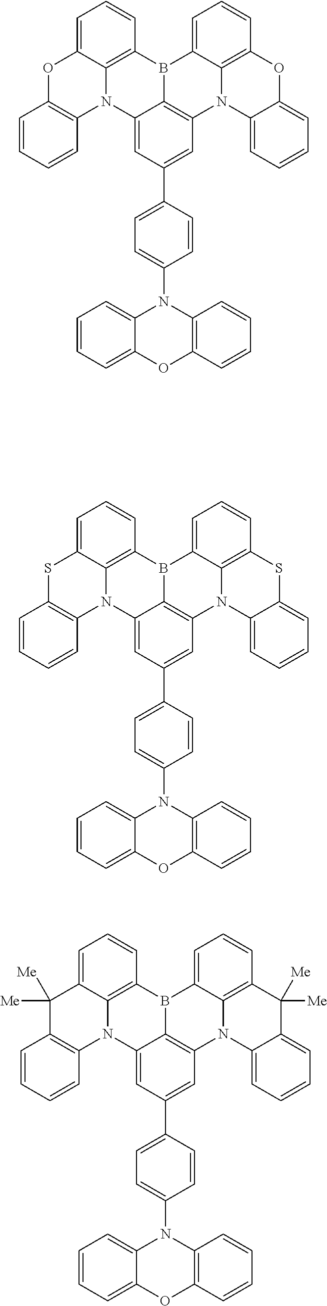 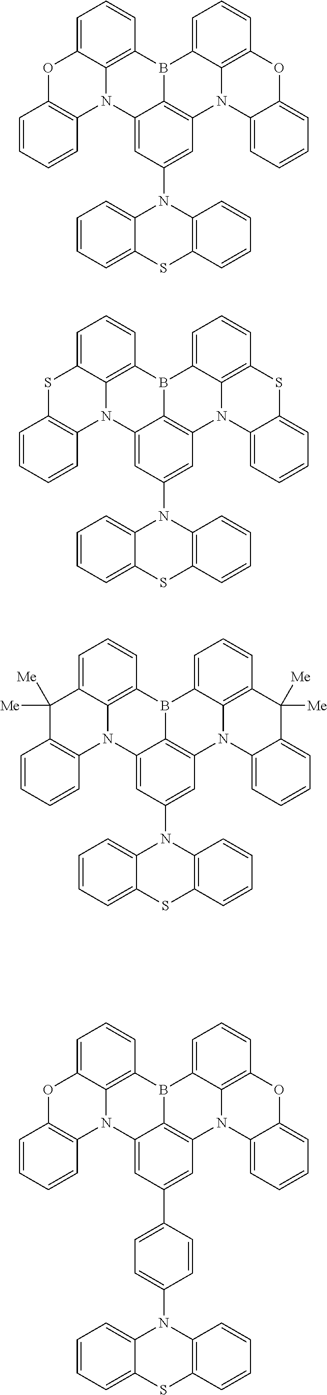 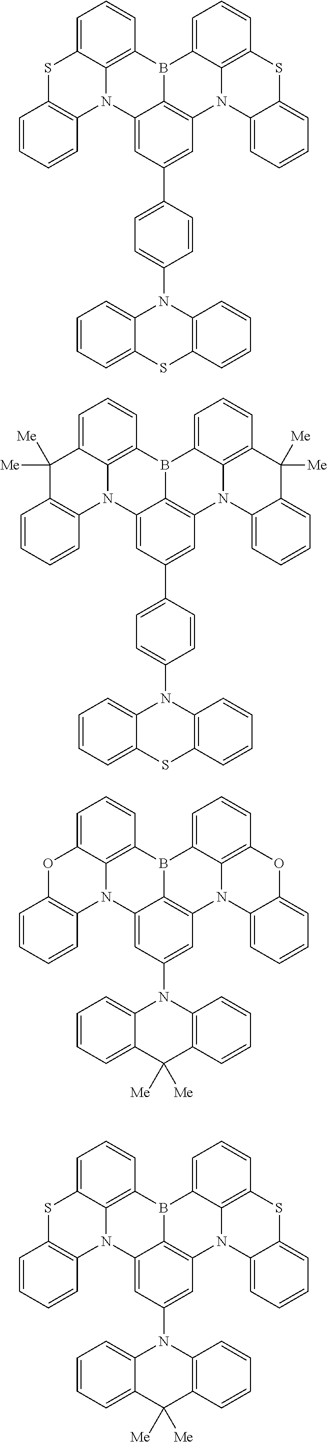 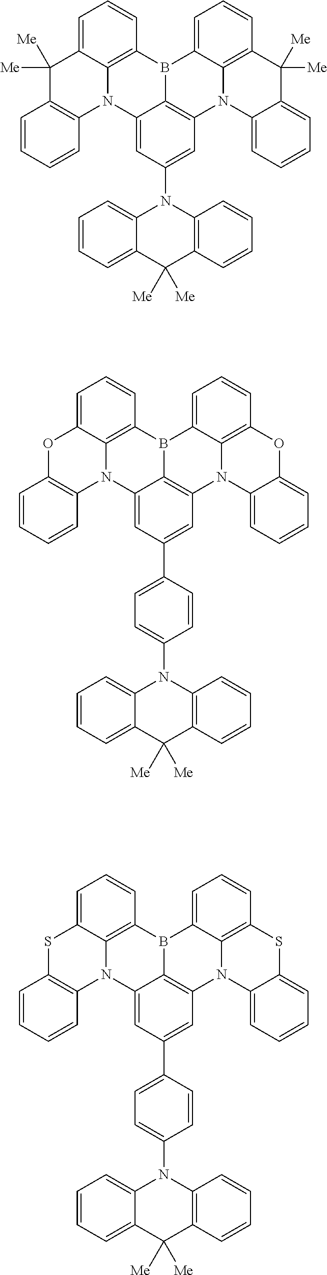 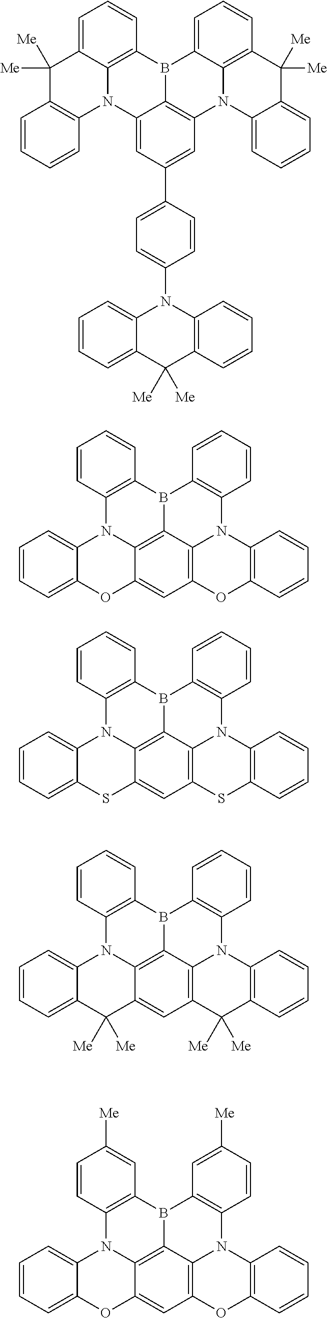 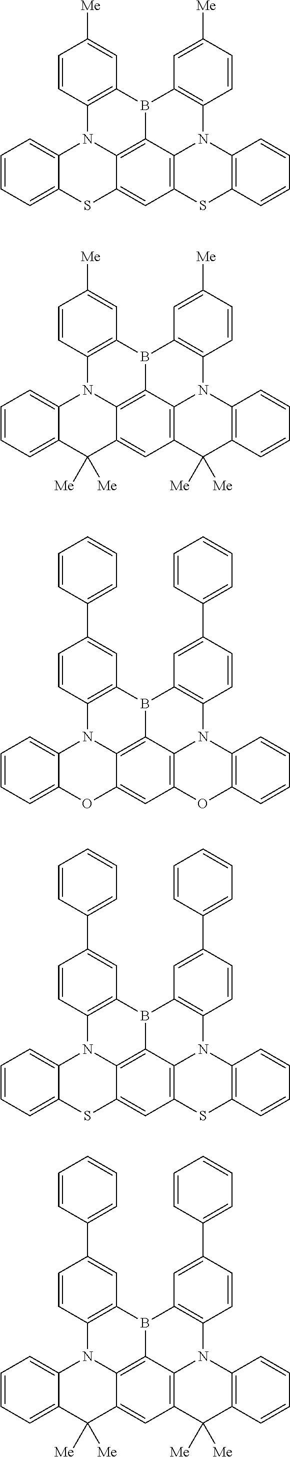 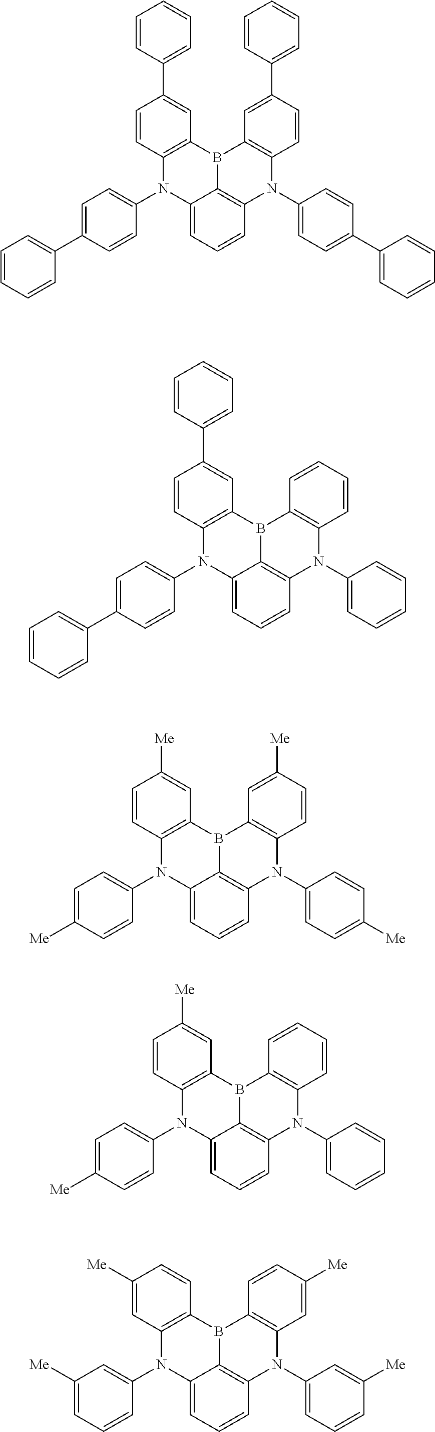 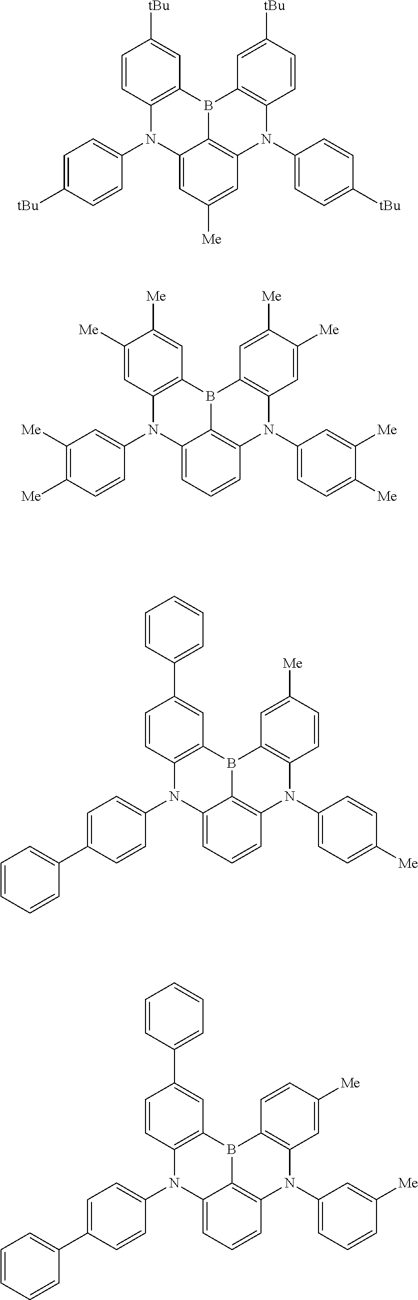 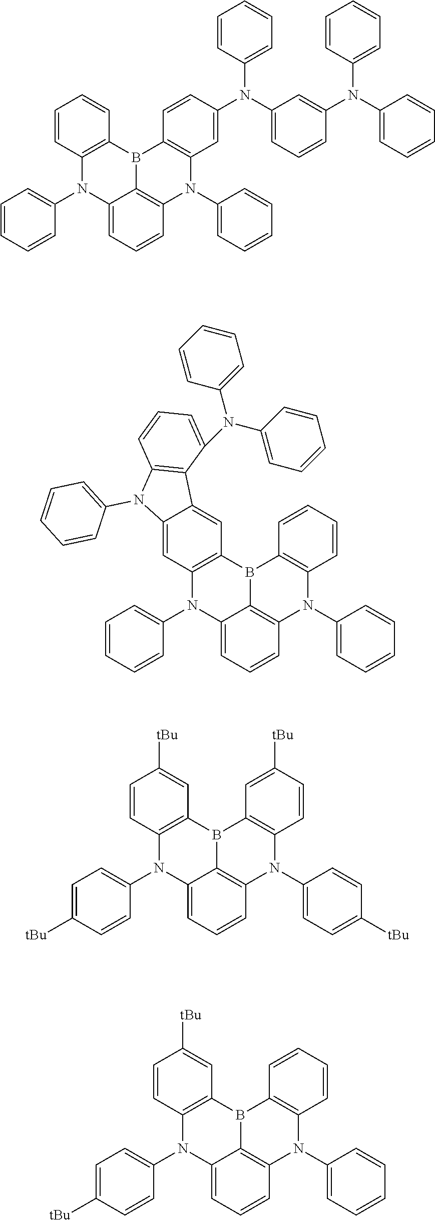 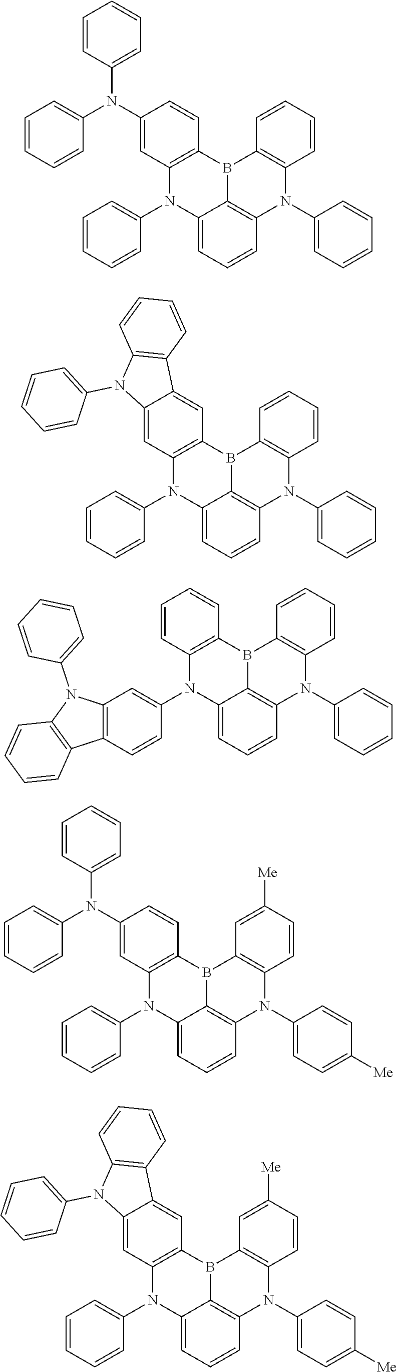 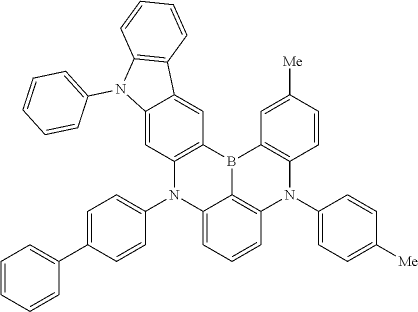 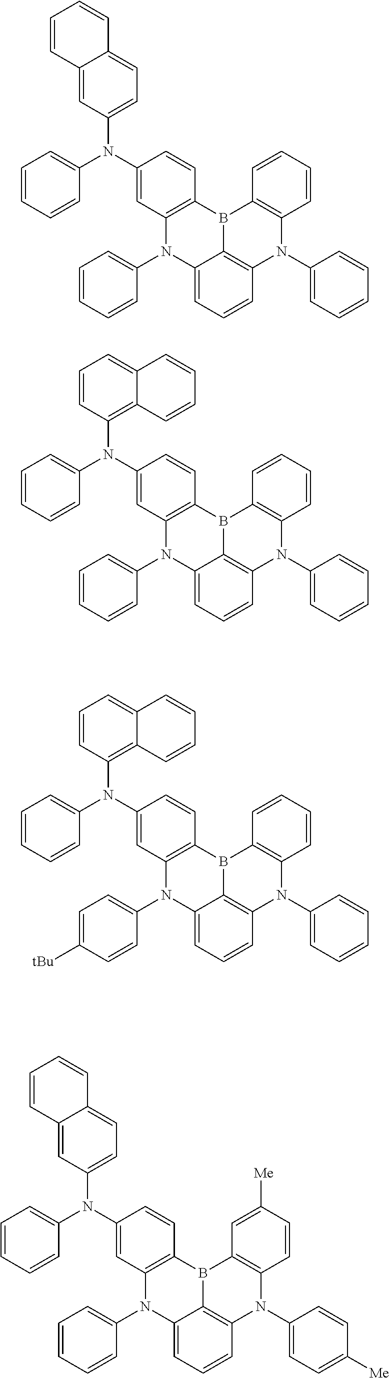 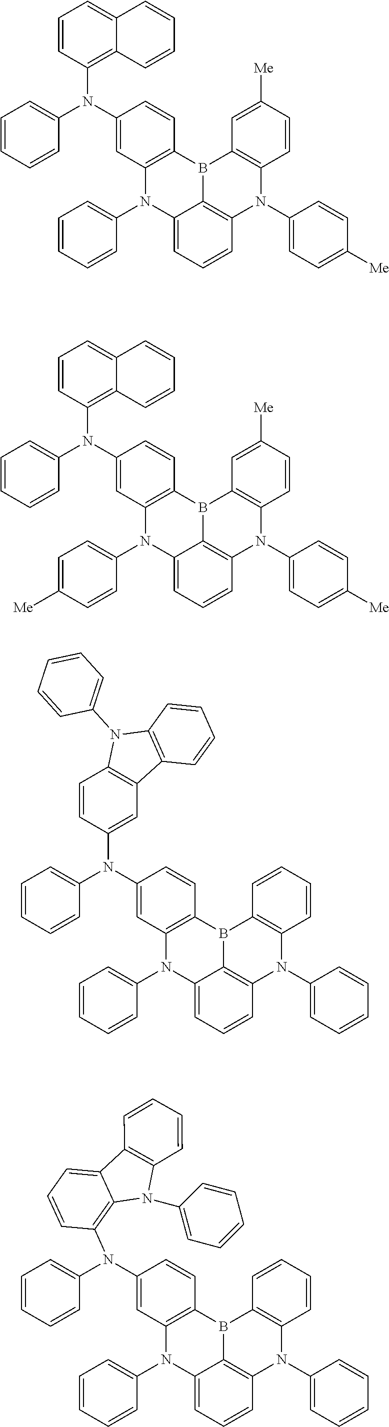 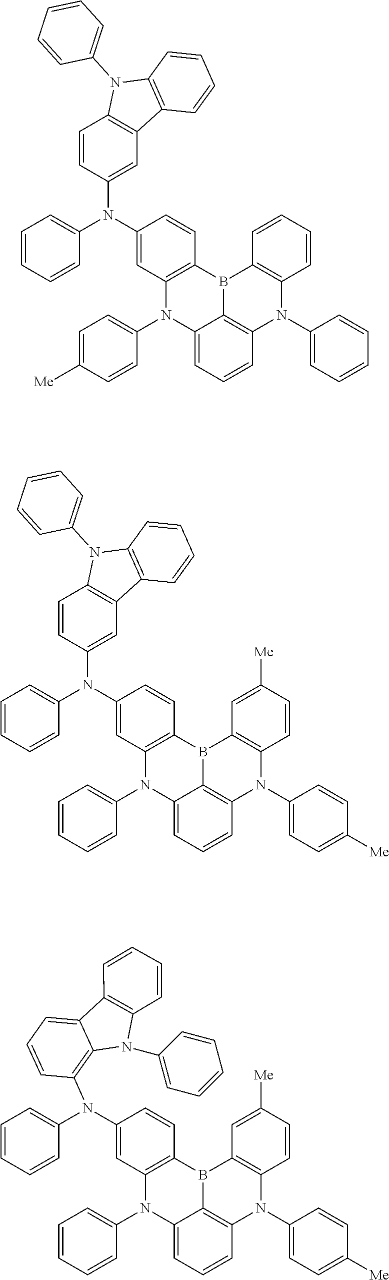 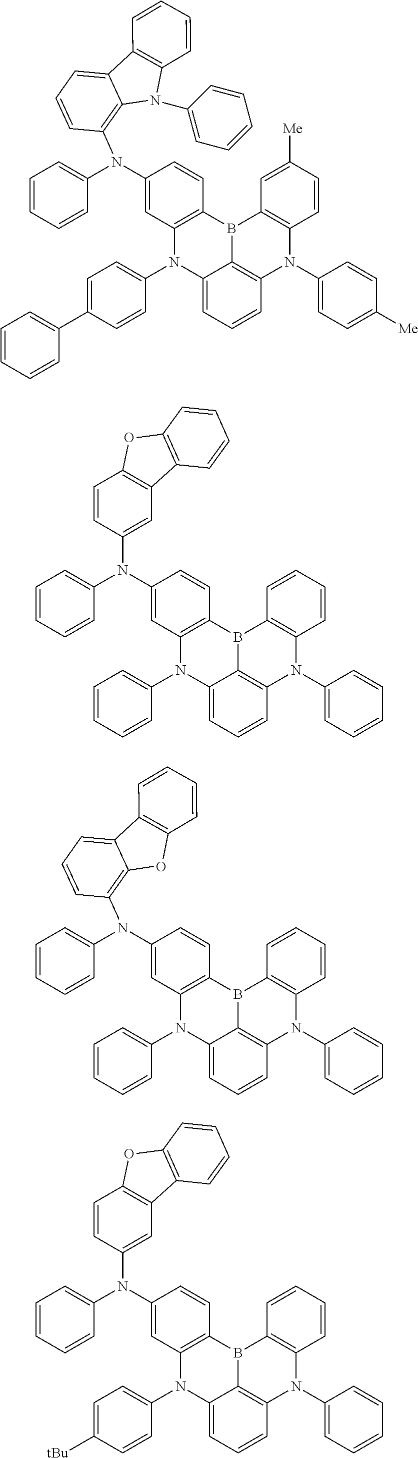 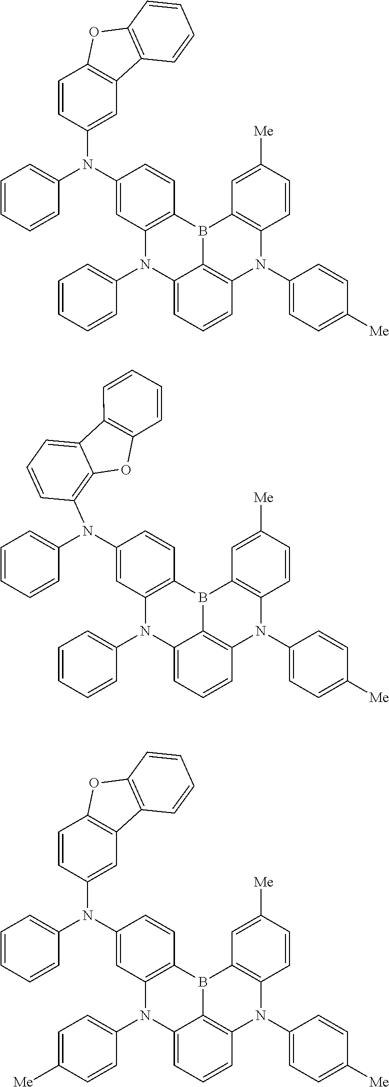 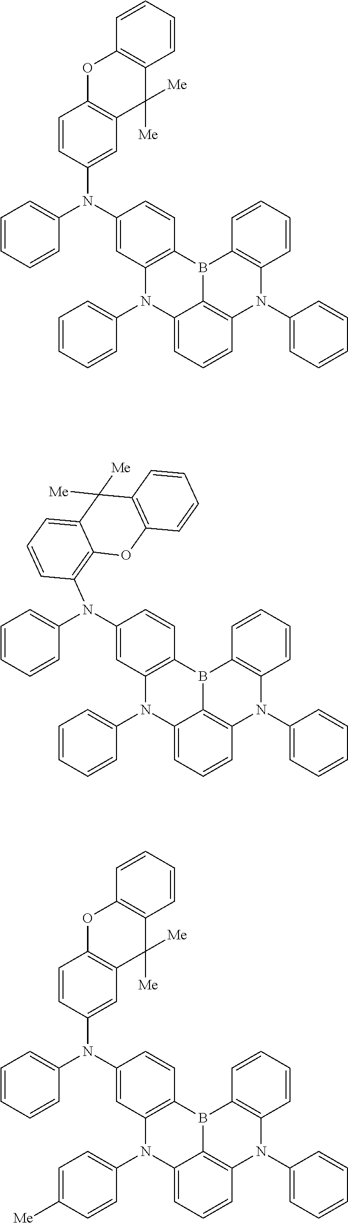 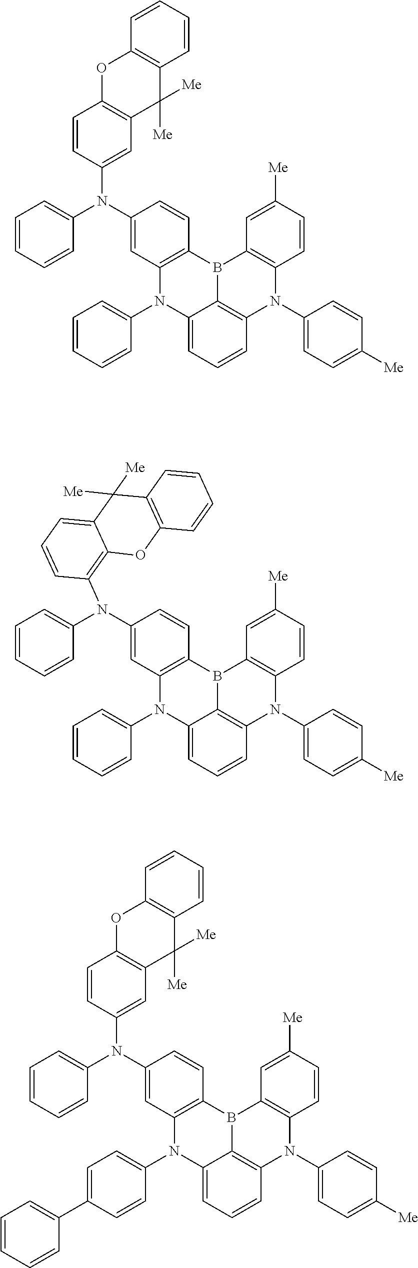 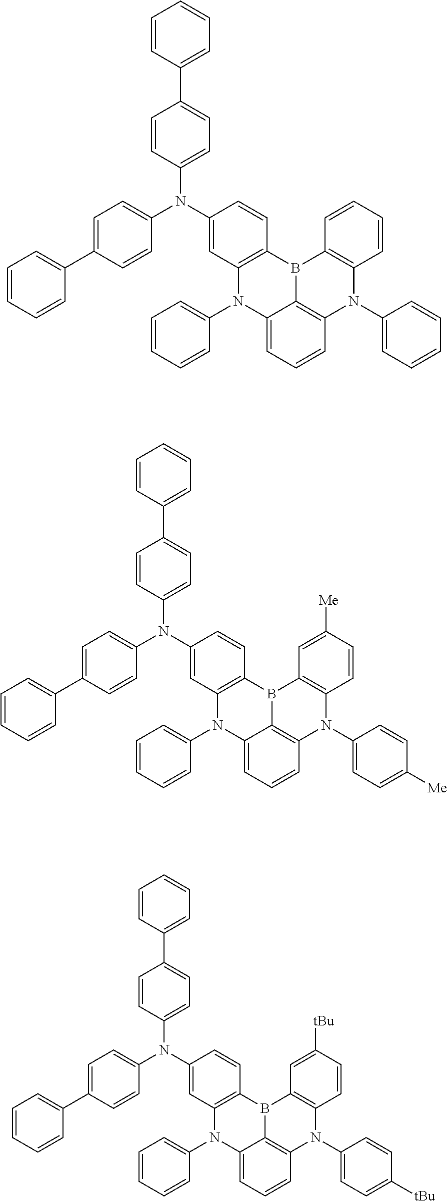 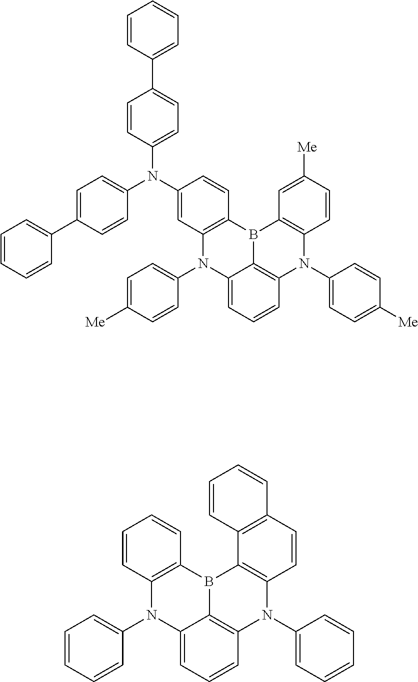 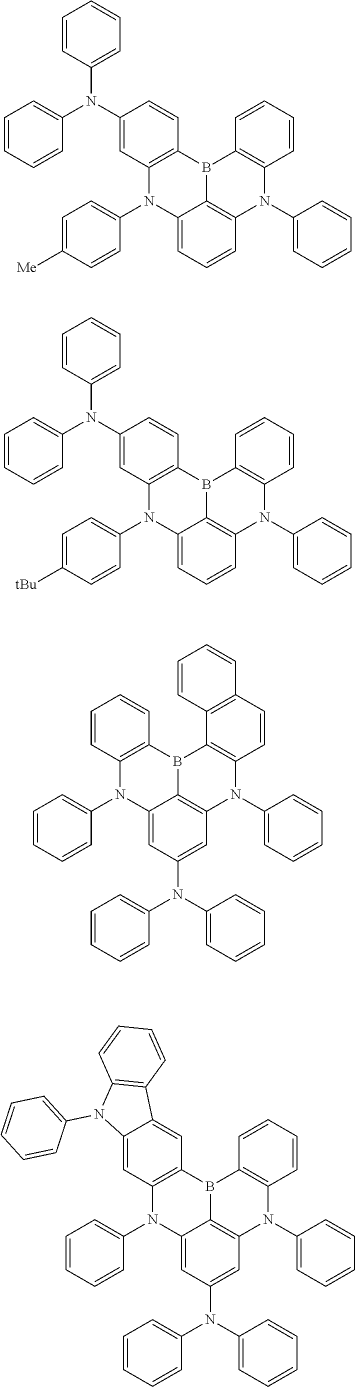 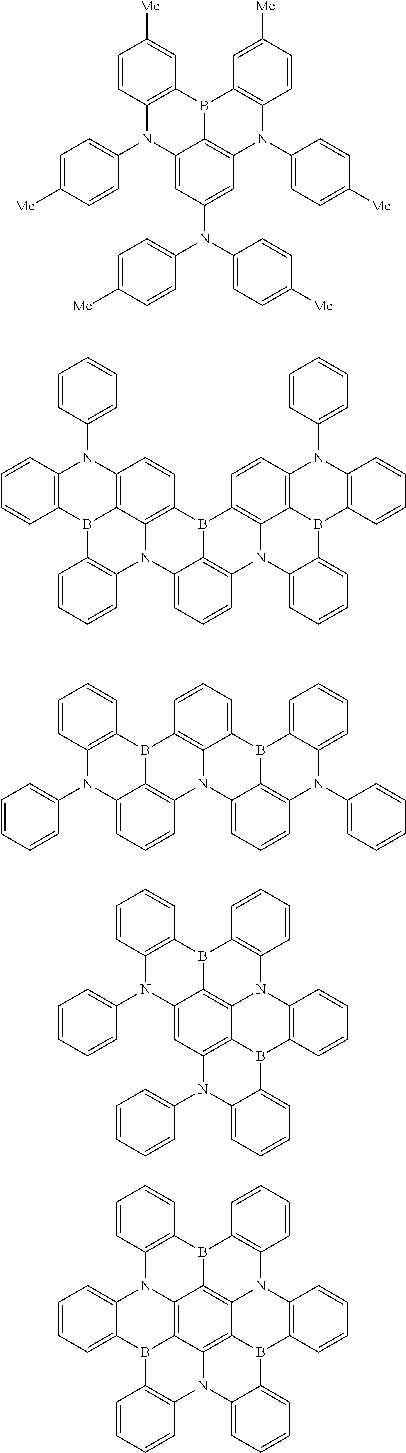 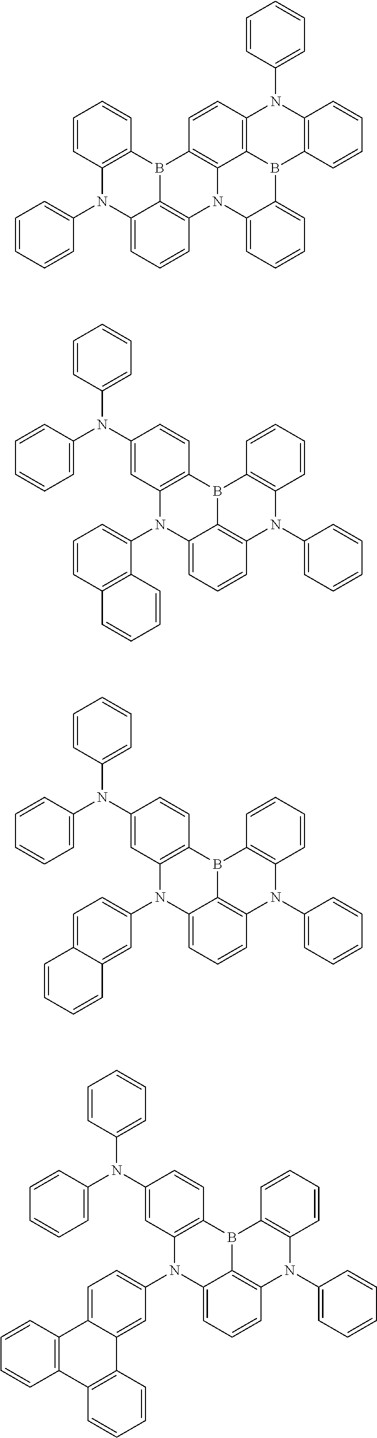 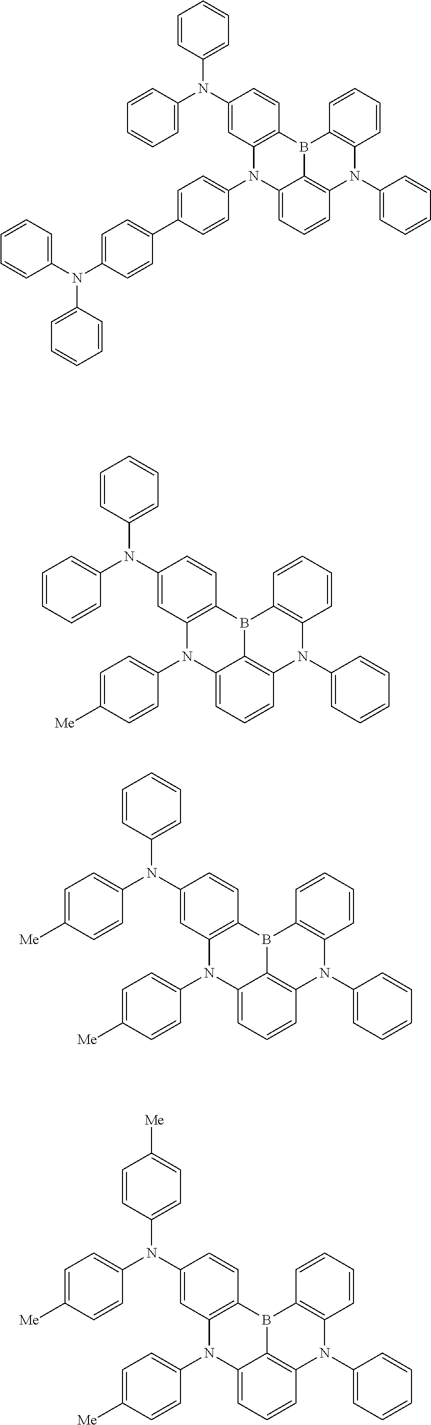 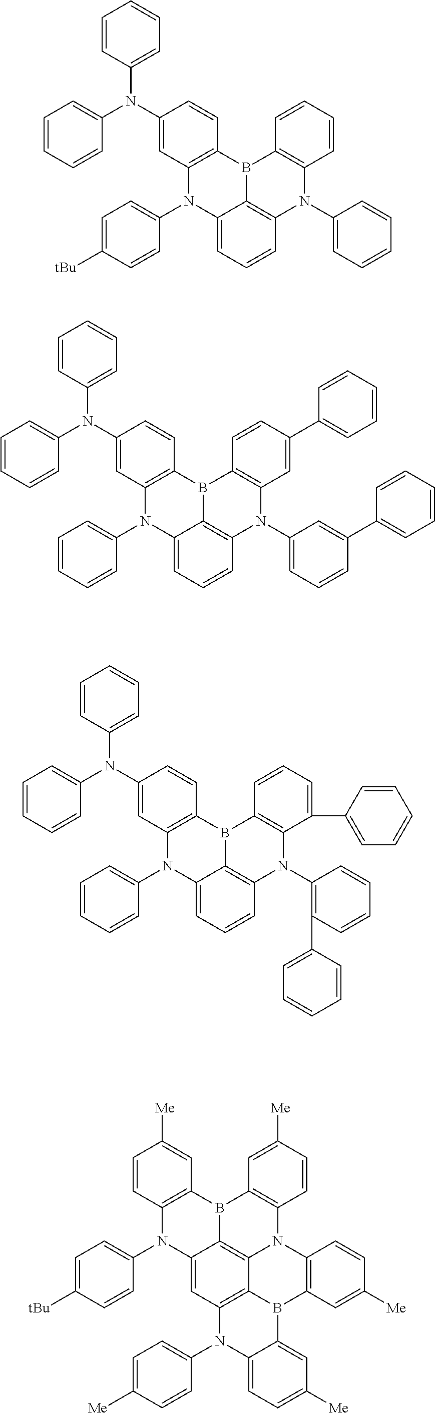 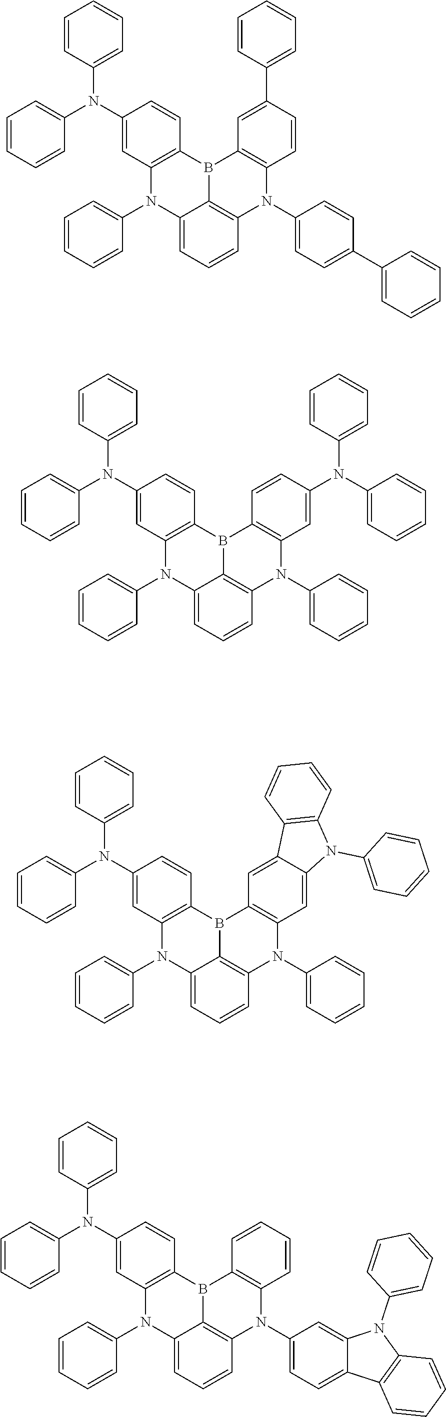 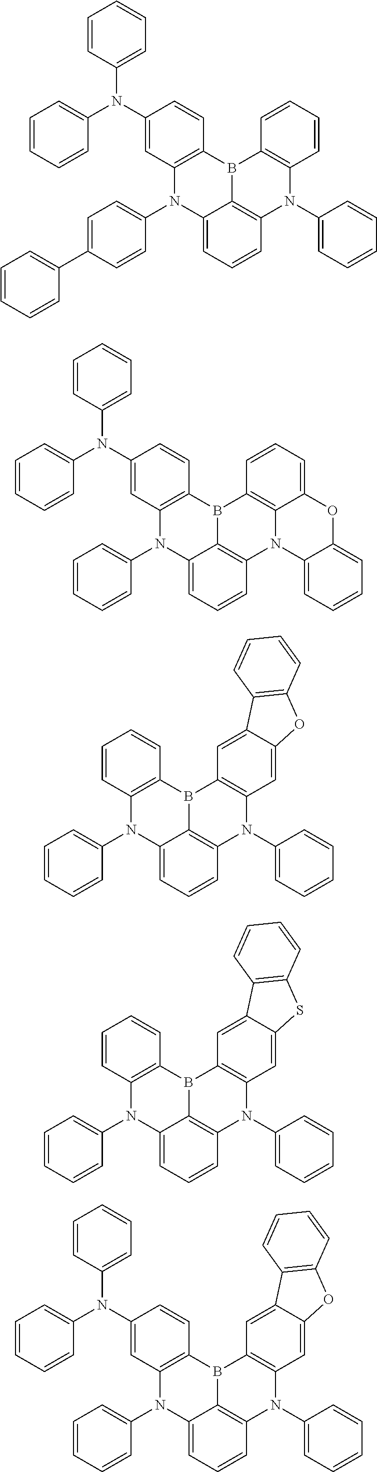 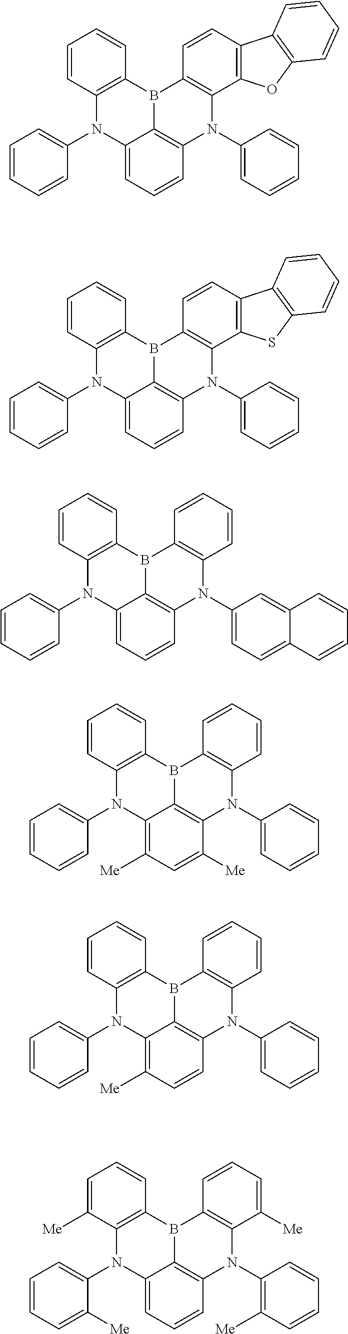 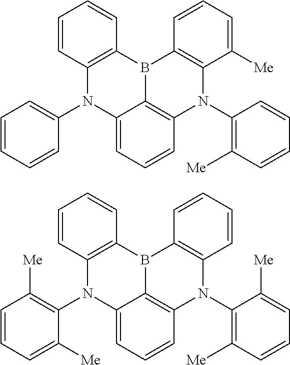 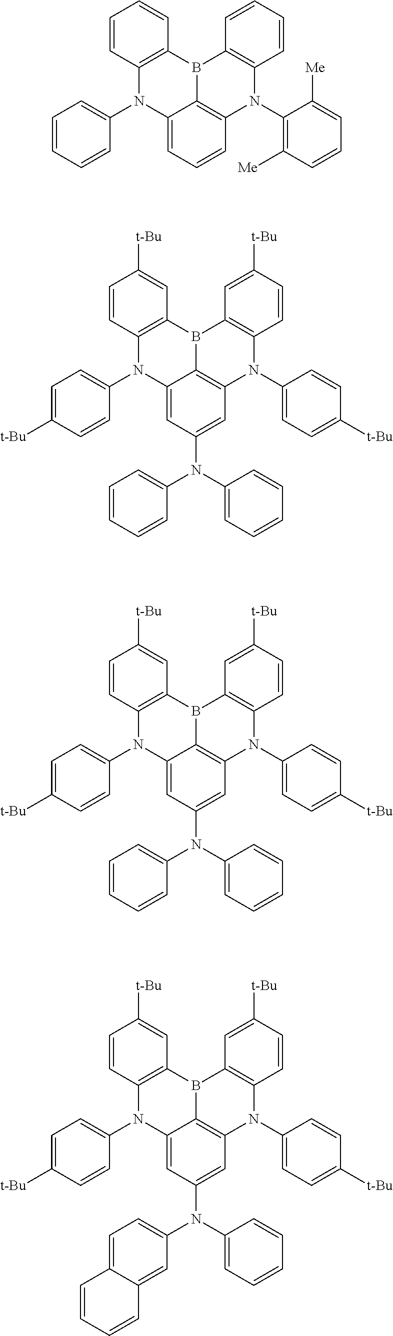 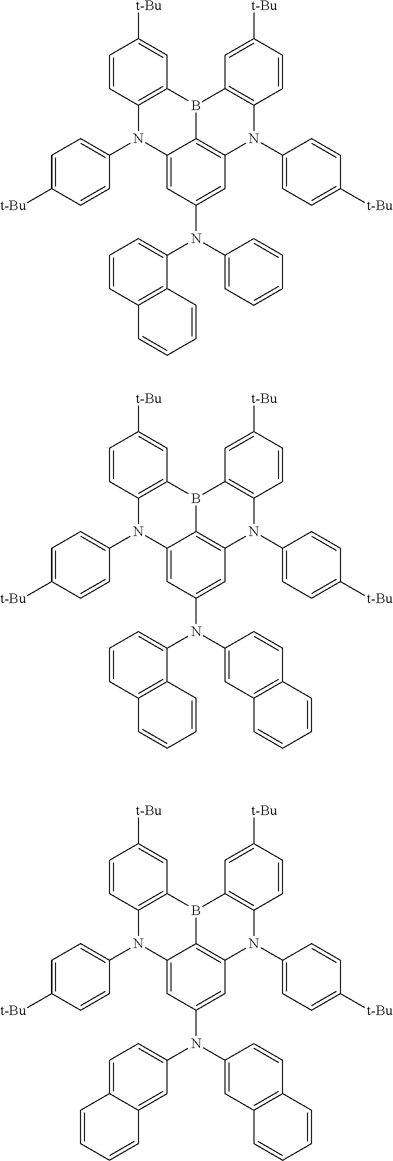 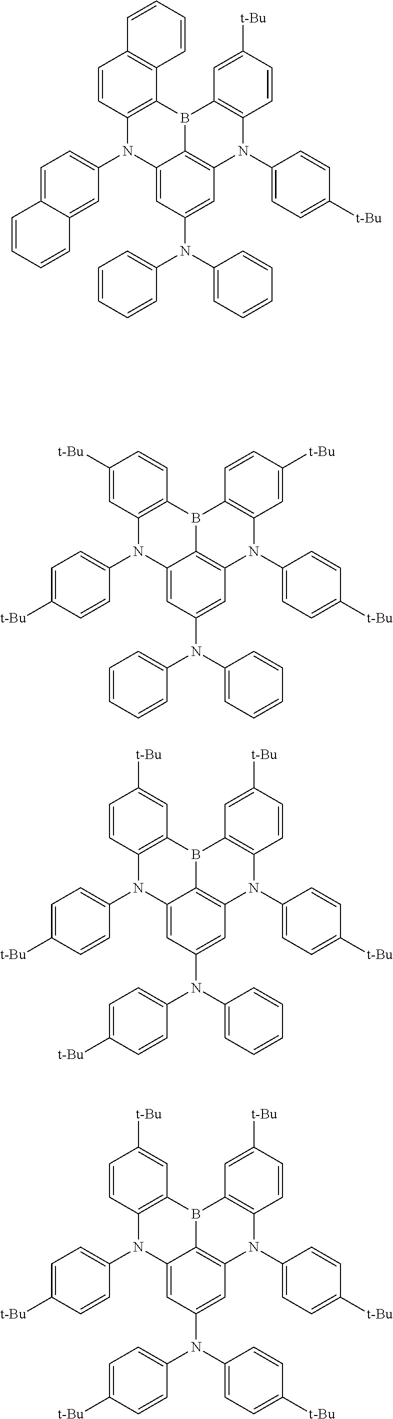 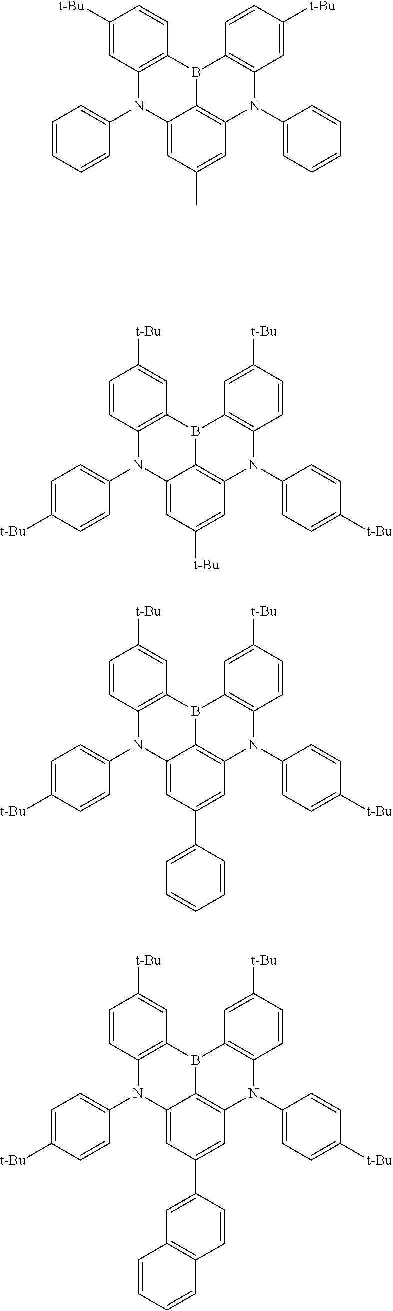 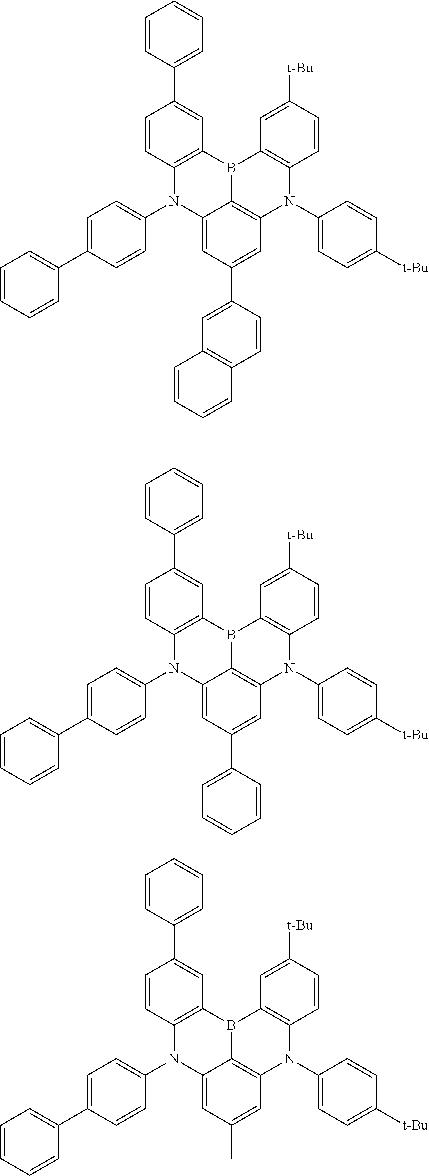 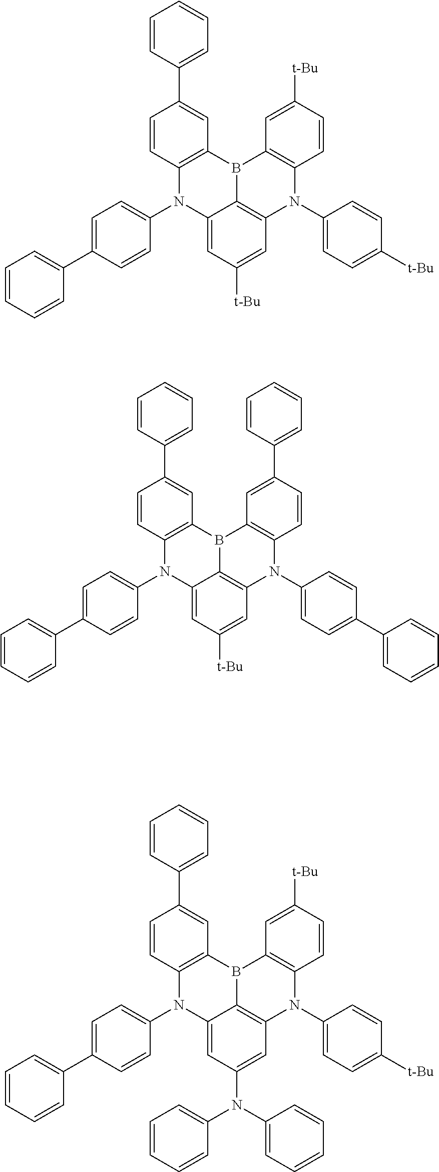 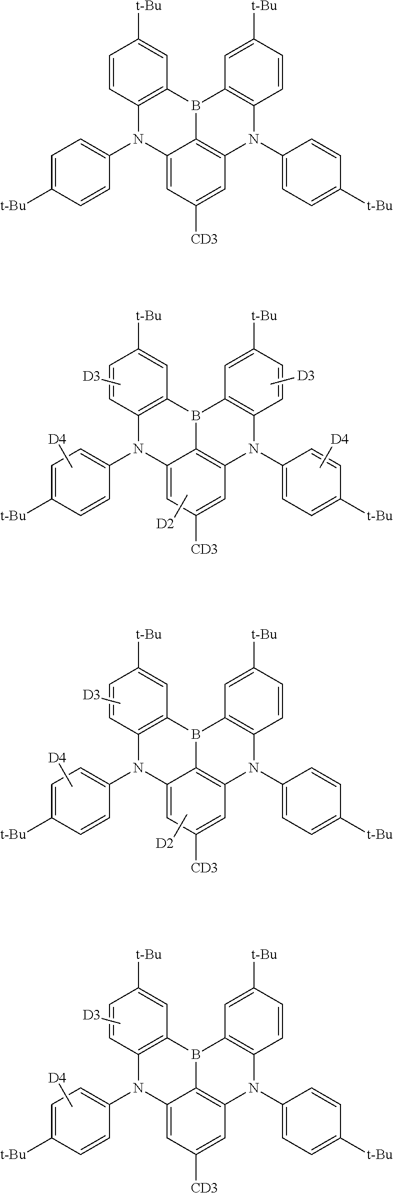 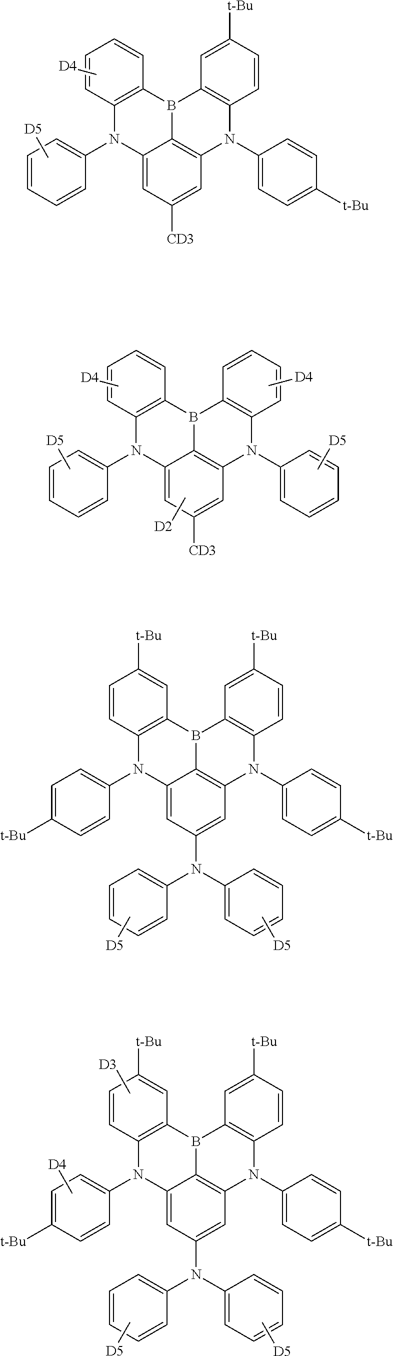 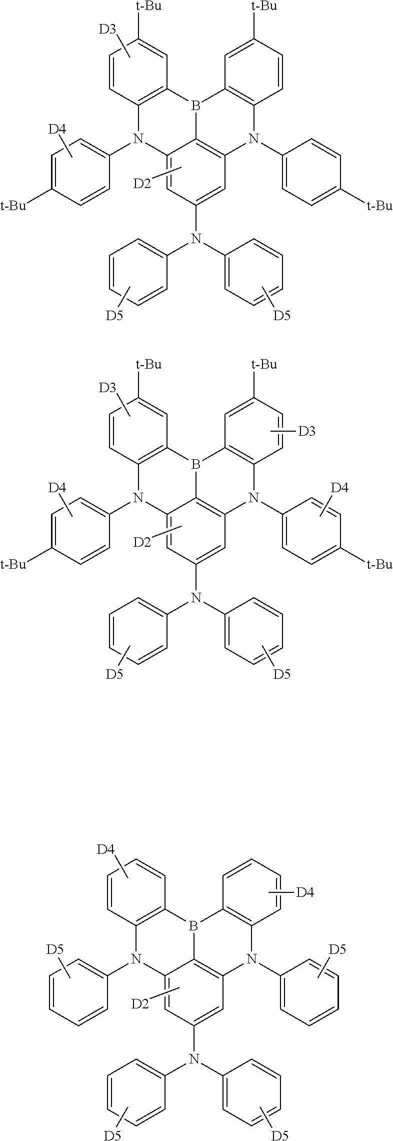 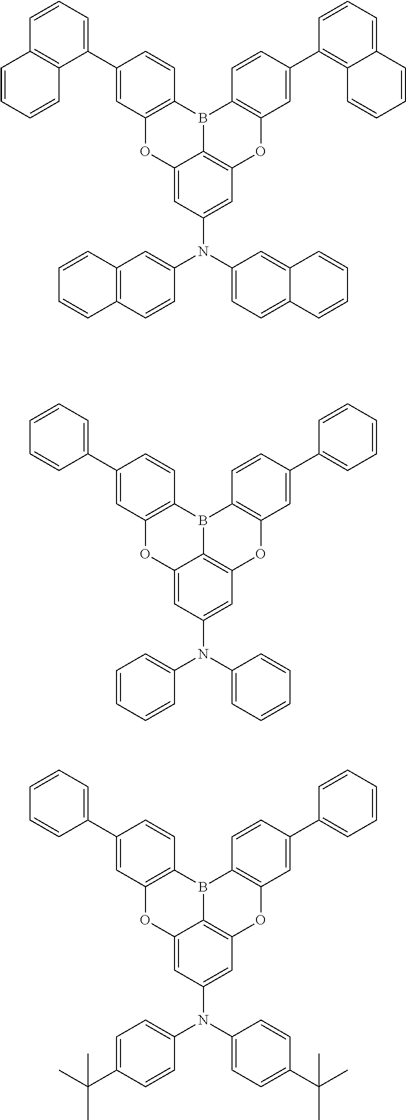 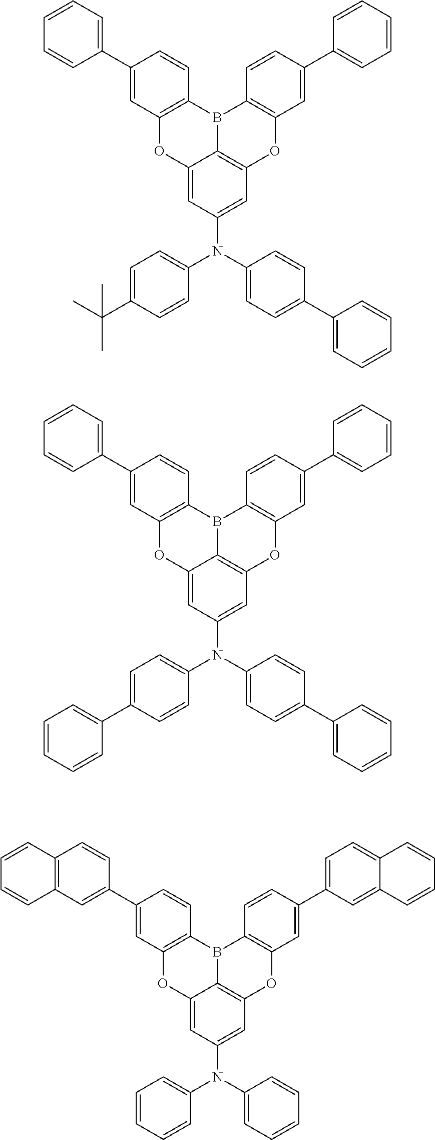 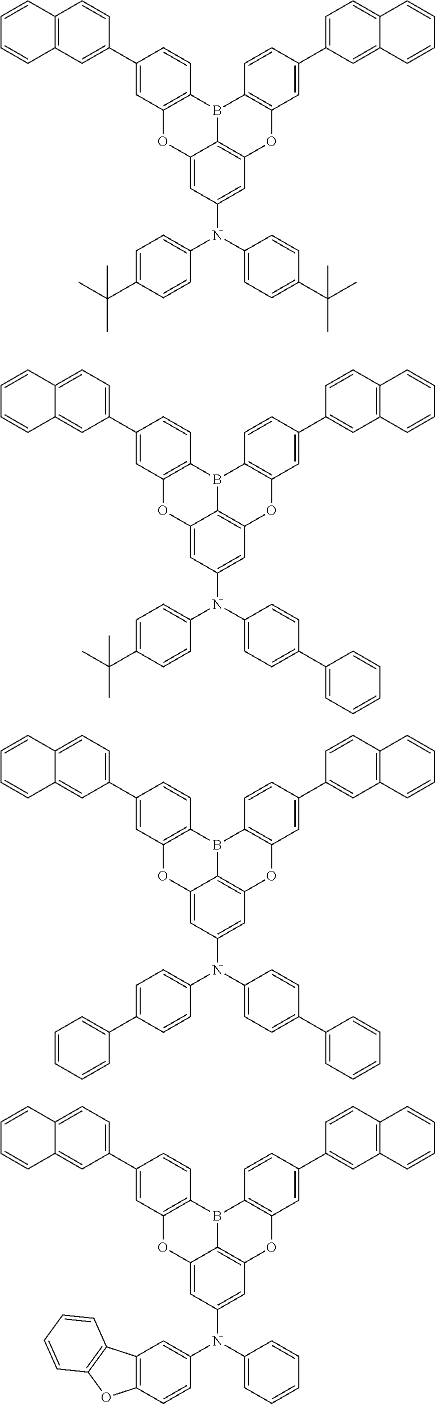 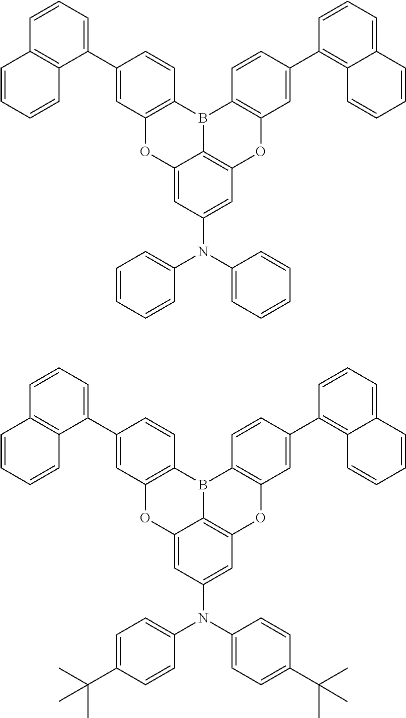 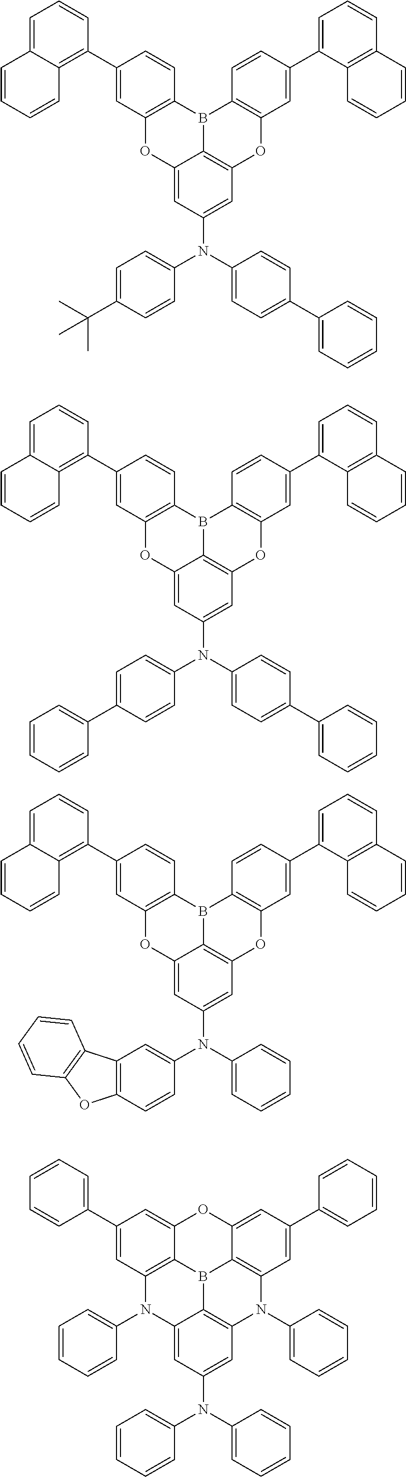 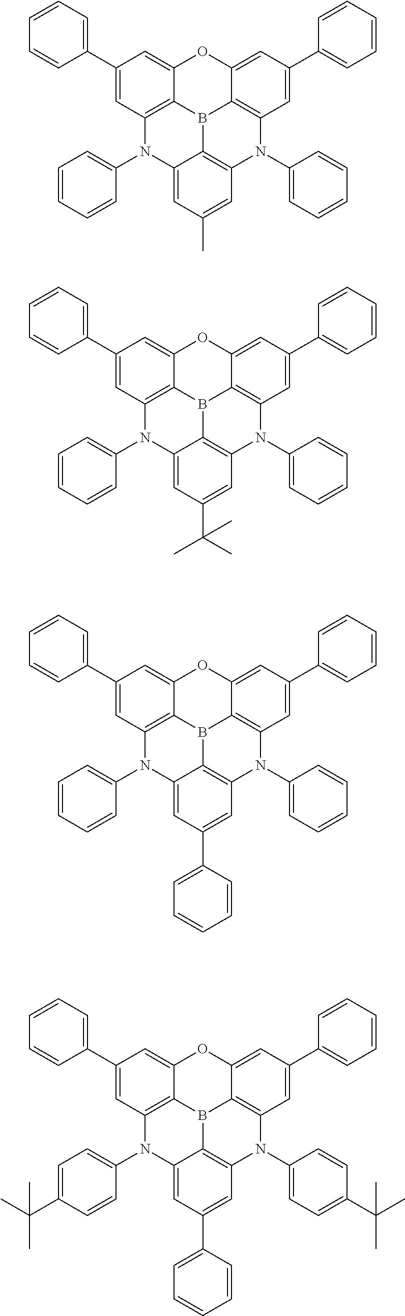 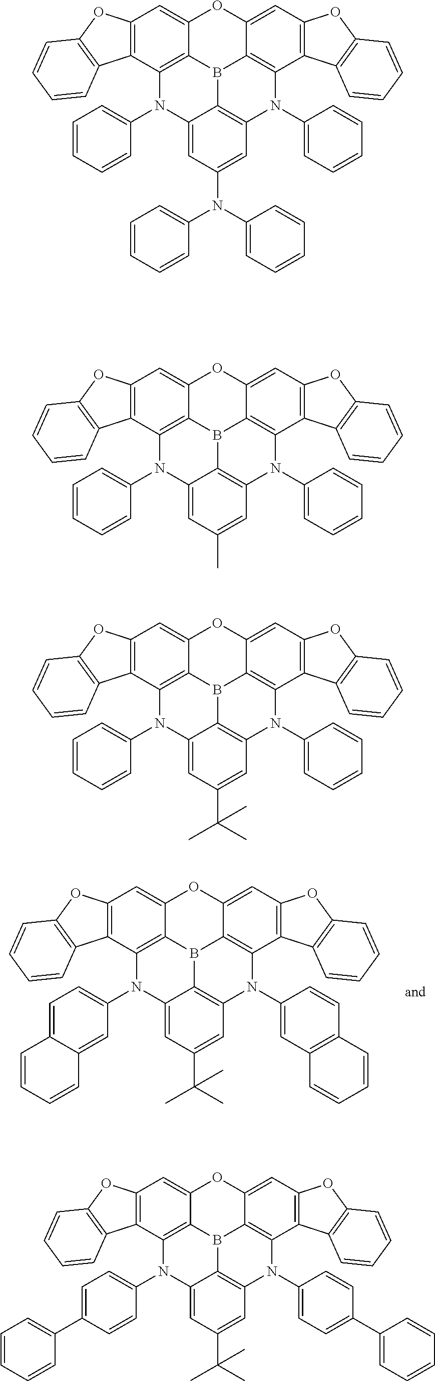
wherein, D2 to D5 represent that 2 to 5 hydrogens are replaced with deuterium, respectively.

8. The plurality of light-emitting materials according to claim 1, wherein the compound represented by formula 1 is a host material, and the compound represented by formula 2 is a dopant material.

9. An organic electroluminescent device comprising an anode, a cathode, and at least one light-emitting layer disposed between the anode and the cathode, wherein the light-emitting layer comprises the plurality of light-emitting materials according to claim 1.

10. The organic electroluminescent device comprising an anode, a cathode, and an organic layer(s) between the anode and the cathode, wherein the organic layer comprises a light-emitting layer and a hole transport zone disposed between the anode and the light-emitting layer, the light-emitting layer comprises the plurality of light-emitting materials according to claim 1, and the hole transport zone comprises a compound represented by the following formula 3: 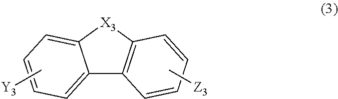
wherein,
X₃ represents NR₄₁, O, S, or CR₄₂R₄₃;
Y₃, Z₃ and R₄₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; and
R₄₂ and R₄₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or R₄₂ and R₄₃ may be linked to each other to form a ring(s).
